(19) United States
(12) Patent Application Publication
(10) Pub. No.: US 2009/0235392 A1
(43) Pub. Date: Sep. 17, 2009

(54) Cytokinin-sensing Histidine Kinases and Methods of Use

(75) Inventors: Robert J Meister, St. Peters, MO (US); Sobhana Sivasankar, Urbandale, IA (US)

Correspondence Address: PIONEER HI-BRED INTERNATIONAL, INC., 7250 N.W. 62ND AVENUE, P.O. BOX 552, JOHNSTON, IA, 50131-0552, US

(73) Assignee: PIONEER HI-BRED INTERNATIONAL, INC., Johnston, IA (US)

(21) Appl. No.: 12/464,527

(22) Filed: May 12, 2009

Related U.S. Application Data
(63) Continuation of application No. 11/273,537, filed on Nov. 14, 2005, now abandoned.
(60) Provisional application No. 60/627,394, filed on Nov. 12, 2004.
(60) Provisional application No. 60/706,787, filed on Aug. 9, 2005.

(51) Int. Cl. C12N 15/82; C12N 5/10; C07K 14/415; C07H 21/04; A01H 1/00; A01H 5/00

(52) U.S. Cl. 800/287; 435/419; 530/370; 536/23.6; 800/278; 800/298; 800/312; 800/314; 800/320; 800/320.1; 800/320.2; 800/320.3

(57) ABSTRACT

Isolated polynucleotides that encode cytokinin-sensing histidine kinase polypeptides, and the encoded polypeptides, are described. Expression cassettes comprising the polynucleotides of the invention and plants and plant cells that are transformed with the polynucleotides are described. Methods of using the cytokinin-sensing histidine kinase polypeptides and polynucleotides to modulate histidine kinase activity and/or histidine kinase levels in plants and plant cells are further described.

### CROSS-REFERENCE

This application is a continuation of U.S. patent application Ser. No. 11/273,537 filed on Nov. 14, 2005, which claims priority to U.S. provisional patent applications 60/627,394, filed Nov. 12, 2004, and 60/706,787, filed Aug. 9, 2005, all of which are hereby incorporated by reference.

### FIELD OF THE INVENTION

The invention is directed to the field of plant molecular biology, particularly to nucleic acid molecules encoding maize histidine kinases and methods of use.

### BACKGROUND OF THE INVENTION

Plants and other organisms use signal transduction systems to perceive environmental and hormonal stimuli and to respond by altering cellular processes through changes in the amount or activity of cascades of gene products. Reversible protein phosphorylation is a key mechanism for intracellular signal transduction in eukaryotic and prokaryotic cells (Urao, et al., (2000) Trends Plant Sci. 5:67-74). Such reversible protein phosphorylation mechanisms involve protein kinases. The protein kinases that are known to be involved in signal transduction are classified into three groups: Serine/threonine-protein kinases, tyrosine-protein kinases, and histidine-protein kinases (Sakakibara, et al., (2000) Plant Mol. Biol. 42:273-278).

While the specific role of histidine-protein kinases (also referred to herein as “histidine kinases”) is only beginning to be revealed for plants, histidine kinases in bacteria are known to play important roles in sensing and transducing a variety of extracellular stimuli (Urao, et al., (2001) Science's STKE doi: 10.1126/stke.2001.109.re18). The signal transduction systems that histidine kinases are involved in are known as two-component signaling systems or His-Asp phosphorelay systems (Sakakibara, et al., (2000) Plant Mol. Biol. 42:273-278). The plant two-component systems are typically made up of three domains: a sensor (hybrid histidine kinase) domain, a histidine containing phosphotransfer domain, and a receiver (response regulator) domain (Sakakibara, et al., (2000) Plant Mol. Biol. 42:273-278; Grefen and Harter, (2004) Planta (Online First) doi: 10.1007/s00425-004-1316-4). In addition to a histidine kinase domain and an invariant histidine residue that is autophosphorylated, the hybrid histidine kinases include a receiver domain usually at the COOH-terminal end that possesses a conserved aspartate residue (Grefen and Harter, (2004) Planta (Online First) doi: 10.1007/s00425-004-1316-4). Instead of transferring the phosphoryl group directly to the response regulator, it is first transferred from the autophosphorylated histidine residue to the conserved aspartate residue of the receiver domain (Grefen and Harter, (2004) Planta (Online First) doi: 10.1007/s00425-004-1316-4).

In plants, the histidine kinases are involved in two-component systems that mediate osmosensing and the perception of the plant hormones ethylene and cytokinin (Urao, et al., (2001) Science's STKE doi: 10.1126/stke.2001.109.re18). Based on the prevalence of genes encoding bacterial-type two-component histidine kinases in the genome of the model plant, Arabidopsis thaliana, bacterial-type, two-component histidine kinases are expected to be involved in signal transduction systems for a variety of environmental and hormonal stimuli. Histidine kinases have been cloned from plants including Arabidopsis thaliana (Chang, et al., (1993) Science 262:539-544; Hua, et al., (1995) Science 269:1712-1714; Kakimoto, (1996) Science 274:982-985; Hua, et al., (1998) Plant Cell 10:1321-1332; Sakai, et al., (1998) Proc. Natl. Acad. Sci. USA 95: 5812-5817), tomato (Payton, et al., (1996) Plant Mol. Biol. 31:1227-1231; Lashbrook, et al., (1998) Plant J. 15:243-252), Rumex palustris (Vriezen, et al., (1997) Plant J. 11:1265-1271), and tobacco (Zhang, et al., (2004) Plant Physiol. 136:2971-2981).

Of the plant histidine kinases, those from Arabidopsis thaliana have been the most thoroughly investigated to date. Sequence analysis of the Arabidopsis thaliana genome has revealed that there are at least sixteen genes encoding putative histidine kinases (Hwang, et al., (2002) Plant Physiol. 129:500-515). Grefen and Harter ((2004) Planta (Online First) doi: 10.1007/s00425-004-1316-40) have indicated that sequence analysis of the entire Arabidopsis genome has revealed the presence of eight canonical histidine kinases. Of the eight canonical Arabidopsis histidine kinases, five (ETR1, ETR2, EIN4, ERS1, and ERS2) function as ethylene receptors and one (CREL) acts as a cytokinin receptor (Urao, et al., (2001) Science's STKE doi: 10.1126/stke.2001.109.re18). Two additional Arabidopsis histidine kinases, CKI1 and CKI2, are also putative cytokinin receptors based on genetic analyses (Urao, et al., (2000) Trends Plant Sci. 5:67-74). The Arabidopsis histidine kinase homologs have been divided into three distinct families: the ethylene receptors, the phytochrome photoreceptors, and the AHK family, which includes a cytokinin receptor (CRE1/AHK4/WOL1) and a putative osmosensing receptor (AtHK1) (Hwang, et al., (2002) Plant Physiol. 129:500-515).

Several publications have alluded to a putative in vivo role for the Arabidopsis CKI2(AtCKI2) in cytokinin signal transduction (Kakimoto, (1996), Grefen and Harter, (2004), Higuchi, et al., (2004)). This histidine kinase clearly lacks the described cytokinin-binding CYCLASES/HISTIDINE KINASES ASSOCIATED SENSORY EXTRACELLULAR (CHASE) domain (Anantharaman and Aravind, (2001); Mougel and Zhulin, (2001); Yamada, et al., (2001)). Data disclosed herein indicate that AtCKI2 functions as an unique intracellular histidine kinase. The carboxyl terminus of the AtCKI2 protein is capable of interaction with the ARABIDOPSIS HISTIDINE PHOSPHOTRANSFER proteins as well as with a hitherto undescribed protein kinase. Analyses of callus tissue from CKI2 mutant and transgenic plants indicate that the protein is capable of positively modulating cytokinin-responsive growth in a manner similar to AHK3.

As the world's human population continues to escalate, new strategies are required for improving agricultural plants to keep up with the demands for increased food production. A better understanding of the components of signal transduction systems will aid in the development of new strategies for improving agricultural crop plants.

### SUMMARY OF THE INVENTION

The present invention provides compositions and methods for modulating plant signal transduction systems. The compositions comprise isolated polynucleotides encoding histidine kinases and isolated polypeptides comprising histidine kinases. Selected isolated polynucleotides of the invention comprise a nucleotide sequence selected from the group consisting of: SEQ ID NOS: 1, 3, 4, 6, 7, 9, 13, 15, 16, 18, 26, 28, 30 and 32; nucleotide sequences encoding the amino acid sequences set forth in SEQ ID NOS: 2, 5, 8, 14, 17, 23, 27 and 31; and fragments and variants thereof. Likewise, selected isolated polypeptides of the invention comprise an amino acid sequence selected from the group consisting of: SEQ ID NOS: 2, 5, 8, 14, 17, 23, 27 and 31; the amino acid sequences encoded by the nucleotide sequences set forth in SEQ ID NOS: 1, 3, 4, 6, 7, 9, 13, 15, 16, 18, 26, 28, 30 and 32; and fragments and variants thereof.

The present invention further provides expression cassettes and transformed plants, plant tissues, plant cells and seeds. The expression cassettes comprise at least one histidine kinase polynucleotide of the invention operably linked to a promoter that drives expression in a plant or cell. The transformed plants, plant tissues, plant cells, and seeds comprise at least one histidine kinase polynucleotide of the invention operably linked to a promoter that drives expression in a plant or cell thereof. In an example of the invention, the histidine kinase polynucleotide of the invention is stably incorporated into the genome of the transformed plants, plant tissues, plant cells, or seeds.

The present invention provides a method of increasing the activity of a polypeptide in a plant comprising providing to the plant a histidine kinase polypeptide of the invention. In one example, the method involves introducing into the plant or at least one cell thereof a histidine kinase polynucleotide of the invention. If desired, the polynucleotide can be stably incorporated into the genome of the plant. In another example, the method involves introducing the histidine kinase polypeptide into a plant or at least one cell thereof. Plants produced by this method have an increased level of histidine kinase activity relative to a plant to which a histidine kinase polypeptide was not provided.

The present invention provides a method for modulating the level of a polypeptide in a plant comprising introducing into a plant a polynucleotide comprising a nucleotide sequence encoding a histidine kinase of the invention. The polynucleotide can further comprise a promoter operably linked to the nucleotide sequence encoding the histidine kinase, said nucleotide sequence being in either the sense or antisense orientation. Depending on the desired outcome, the method can be used to increase or decrease the level of a histidine kinase polypeptide in a plant or plant part.

### BRIEF DESCRIPTION OF THE DRAWINGS

FIG. 1 is an alignment of hybrid-type receptor histidine kinases. Sequence homologs of CKI2 from Zea mays (ZmCKI2, SEQ ID NO: 8), Oryza sativum (OsCKI2, SEQ ID NO: 23) and Arabidopsis thaliana (AtCKI2, SEQ ID NO: 14) were aligned with putative cytokinin receptors from Zea mays (ZmHK1, SEQ ID NO: 22; ZmHK2, SEQ ID NO: 5; ZmHK3, SEQ ID NO: 31 and ZmCRE1, SEQ ID NO: 1) and Arabidopsis thaliana (AtCRE1, SEQ ID NO: 21; AtAHK2, SEQ ID NO: 19 and AtAHK3, SEQ ID NO: 20) using a BLOSUM62 matrix of the CLUSTALW software. The ZmCRE1 sequence is full-length sequence based on a proprietary EST clone, and sequence derived from a BAC screen. The conserved residues of the histidine kinase (overline) and response regulator (double overline) domains are highlighted (Sheets 3-5) and the putative sites of phosphorylation are indicated with an asterisk (Hwang, et al., (2002) Plant Physiol. 129:500-515). The amino terminal regions of the CKI2 homologs (identical residues bold and italicized; Sheets 1-3) contain a PAS core-like domain (dotted underline) (Taylor and Zhulin, (1999) Microbiol. Mol. Biol. Rev. 63-479-506) adjacent to a putative alpha helical region. In contrast, the putative cytokinin receptors contain the hormone-binding CHASE domain (Anatharaman and Aravind, (2001) Trends Biochem. Sci. 26:579-582) (identical residues are bold and underlined; Sheets 1-3). Non-conserved regions (inverted text) include the extreme amino termini of the CHASE domain-containing proteins, residues flanked by the N and G1 boxes of the histidine kinase domain, and the residues between the conserved histidine kinase and response regulator domains.

FIG. 2 is a ClustalW alignment of CKI2 amino acid sequences of Arabidopsis and rice. Identical residues are shaded and putative phosphorylated residues are shown in inverted text. AT, Arabidopsis thaliana; OS, Oryza sativum.

FIG. 3. Putative functional domains of AtCKI2. AtCKI2 contains signature residues of both histidine kinase (A) and response regulator (B) domains (identical residues in at least four sequences are shown in inverted text), including the phosphorylated histidine and aspartate (asterisk) residues. (C) The CKI2 amino terminus has a region of sequence identity to signature residues of the PAS core domain (asterisk). Three adjacent regions (dotted overline) have a repeating hydrophobic residue motif (grey highlight). AT, Arabidopsis thaliana; OS, Oryza sativum; SC, Saccharomyces cerevisiae; two dots represent a variable number of amino acids that are omitted for clarity; the H, N, G1, F and G2 motifs of histidine kinases and four response regulator motifs have been previously described (Stock, et al., (2000)). AtETR1 sequence shown is from NCBI accession AAA 70047 (SEQ ID NO: 33). ScSLN1 sequence shown is from NCBI accession CAA 86131 (SEQ ID NO: 34).

FIG. 4. Arabidopsis histidine kinase insertional mutants. The genomic insertion site of the T-DNA left border sequence was determined for (A) cki2-2 (SEQ ID NO: 35), (B) ahk3-4 (SEQ ID NO: 20) and (C) ahk1-1 (SEQ ID NO: 25). In cki2-2 and ahk3-4, the in-frame translational product of the left border sequence, with a period representing a stop codon, is shown in inverted text. The cki2-2 insertion is located between motif II and III of the response regulator domain. The ahk3-4 insertion is within the G2 box of the histidine kinase domain and the ahk1-1 insertion is adjacent to the G2 box, located between the histidine kinase and response regulator domains.

FIG. 5. Phylogenetic tree of Arabidopsis and maize histidine kinases. The boxed names represent the new maize sequences identified. The alignment was done using a BLOSUM matrix of the CLUSTALW software.

### SEQUENCE LISTING

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleic acid sequence is shown, but the complementary strand is understood to be included by any reference to the displayed strand. The amino acid sequences follow the standard convention of beginning at the amino terminus of the sequence and proceeding forward (i.e., from left to right in each line) to the carboxy terminus.

The coding sequences disclosed and/or referred to herein may or may not include a stop codon. If desired, a stop codon can be added to any coding sequence. Such stop codons include, for example, TAA, TAG and TGA.

SEQ ID NO: 1 sets forth the nucleotide sequence encoding the maize ZmCRE1 protein. The protein coding sequence is from nucleotide 1-2901.

SEQ ID NO: 2 sets forth the ZmCRE1 amino acid sequence that is encoded by SEQ ID NO: 1.

SEQ ID NO: 3 sets forth the nucleotide sequence for the ZmCRE1 coding region of SEQ ID NO: 1. Nucleotides 1-2901 of SEQ ID NO: 3 correspond to nucleotides 1-2901 of SEQ ID NO: 1.

SEQ ID NO: 4 sets forth the nucleotide sequence encoding the maize ZmHK2 protein. The protein coding sequence is from nucleotide 1-3021.

SEQ ID NO: 5 sets forth the ZmHK2 amino acid sequence that is encoded by SEQ ID NO: 4.

SEQ ID NO: 6 sets forth the nucleotide sequence for the ZmHK2 coding region of SEQ ID NO: 4. Nucleotides 1-3021 of SEQ ID NO: 6 correspond to nucleotides 1-3021 of SEQ ID NO: 4.

SEQ ID NO: 7 sets forth the nucleotide sequence encoding the maize ZmCKI2 protein. The protein coding sequence is from nucleotide 1-2895.

SEQ ID NO: 8 sets forth the ZmCKI2 amino acid sequence that is encoded by SEQ ID NO: 7.

SEQ ID NO: 9 sets forth the nucleotide sequence for the ZmCKI2 coding region of SEQ ID NO: 7. Nucleotides 1-2895 of SEQ ID NO: 9 correspond to nucleotides 1-2895 of SEQ ID NO: 7.

SEQ ID NO: 10 sets forth the nucleotide sequence encoding the partial-length coding sequence of the maize ZmCRE1 protein.

SEQ ID NO: 11 sets forth the ZmCRE1 amino acid sequence that is encoded by SEQ ID NO: 10.

SEQ ID NO: 12 sets forth the nucleotide sequence that encodes the ZmCRE1 partial-length amino acid sequence of SEQ ID NO: 11. Nucleotides 1-1788 of SEQ ID NO: 12 correspond to nucleotides 3-1790 of SEQ ID NO: 10.

SEQ ID NO: 13 sets forth a nucleotide sequence that encodes AtCKI2 (NCBI accession AAZ98829).

SEQ ID NO: 14 sets forth the AtCKI2 amino acid sequence that is encoded by SEQ ID NO: 13.

SEQ ID NO: 15 sets forth the nucleotide sequence for the AtCKI2 coding region of SEQ ID NO: 13. Nucleotides 1 to 2769 of SEQ ID NO: 15 correspond to nucleotides 378-3146 of SEQ ID NO: 13.

SEQ ID NO: 16 sets forth the nucleotide sequence that encodes AtAPK3.

SEQ ID NO: 17 sets forth the AtAPK3 amino acid sequence that is encoded by SEQ ID NO: 16.

SEQ ID NO: 18 sets forth the nucleotide sequence for the AtAPK3 coding region of SEQ ID NO: 16. Nucleotides 1 to 1428 of SEQ ID NO: 18 correspond to nucleotides 1 to 1428 of SEQ ID NO: 16.

SEQ ID NO: 19 sets forth the amino acid sequence of AtAHK2.

SEQ ID NO: 20 sets forth the amino acid sequence of AtAHK3.

SEQ ID NO: 21 sets forth the amino acid sequence of AtCRE1.

SEQ ID NO: 22 sets forth the amino acid sequence of ZmHK1.

SEQ ID NO: 23 sets forth the amino acid sequence of OsCKI2.

SEQ ID NO: 24 sets forth the amino acid sequence of AtCKI1.

SEQ ID NO: 25 sets forth the amino acid sequence of AtHK1.

SEQ ID NO: 26 sets forth the nucleotide sequence that encodes ZmCKI1.

SEQ ID NO: 27 sets forth the amino acid sequence that is encoded by SEQ ID NO: 26.

SEQ ID NO: 28 sets forth the nucleotide sequence for the ZmCKI1 coding region of SEQ ID NO: 26. Nucleotides 1 to 3180 of SEQ ID NO: 28 correspond to nucleotides 1 to 3180 of SEQ ID NO: 26.

SEQ ID NO: 29 sets forth an AtCKI2 promoter sequence.

SEQ ID NO: 30 sets forth the nucleotide sequence that encodes ZmHK3.

SEQ ID NO: 31 sets forth the amino acid sequence that is encoded by SEQ ID NO: 30.

SEQ ID NO: 32 sets forth the nucleotide sequence for the ZmHK3 coding region of SEQ ID NO: 30. Nucleotides 1 to 3606 of SEQ ID NO: 32 correspond to nucleotides 1 to 3606 of SEQ ID NO: 30.

SEQ ID NO: 33 sets forth the amino acid sequence of AtETR1 (AAA 70047).

SEQ ID NO: 34 sets forth the amino acid sequence of ScSLN1 (CAA 86131).

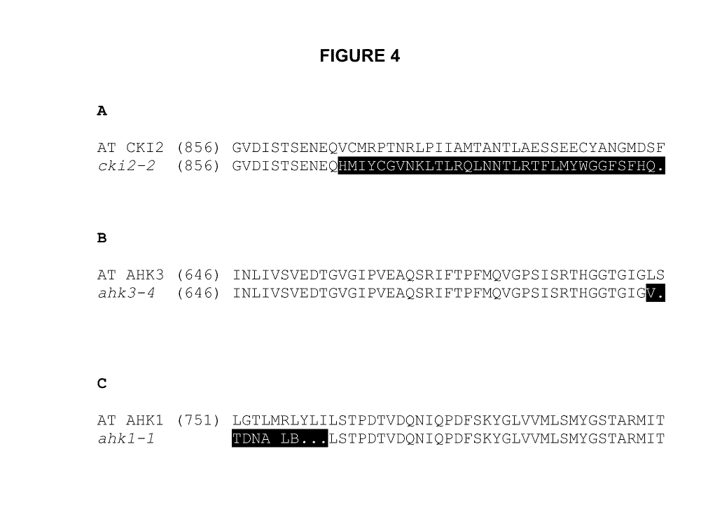

SEQ ID NO: 35 sets forth the cki2-2 insertional mutant of FIG. 4A.

### DETAILED DESCRIPTION OF THE INVENTION

Polynucleotides encoding histidine kinases, the histidine kinase polypeptides encoded thereby and methods of using the same, are provided. Such histidine kinases, which may also be referred to as histidine-protein kinases, are known to be involved in two-component signal transduction systems in plants and other eukaryotic organisms and prokaryotes. In bacteria, histidine kinases are known to be involved in signal transduction in response to extracellular signals including chemotactic factors, changes in osmolarity and nutrient deficiency (Urao, et al., (2000) Trends Plant Sci. 5:67-74). In plants, histidine kinases are known to be involved in osmosensing, ethylene perception, and cytokinin signaling (Sakakibara, et al., (2000) Plant Mol. Biol. 42:273-278; Urao, et al., (2000) Trends Plant Sci. 5:67-74). Thus, the histidine kinase polynucleotides and polypeptides of the present invention will find use in methods for modulating signal transduction pathways in plants so as to alter the response of plants, particularly agricultural crop plants, to environmental and/or hormonal stimuli.

Compositions of the invention include maize histidine kinase polynucleotides and polypeptides that are involved in two-component signal transduction systems in plants. In particular, the present invention provides for isolated polynucleotides comprising nucleotide sequences encoding the amino acid sequences shown in SEQ ID NOS: 2, 5, 8, 14, 17, 27 and 31, including the polynucleotides of SEQ ID NOS: 1, 3, 4, 6, 7, 9, 13, 15, 16, 18, 26, 28, and 32; the polypeptides encoded thereby; the amino acid sequence of SEQ ID NO: 23; and fragments and variants thereof.

The invention encompasses isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various examples, the isolated polynucleotide can contain less than about 5 kb, 4 kb, 3 kb, 2 kb, 1 kb, 0.5 kb or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5% or 1% (by dry weight) of contaminating protein. When the protein of the invention or biologically active portion thereof is recombinantly produced, optimally culture medium represents less than about 30%, 20%, 10%, 5% or 1% (by dry weight) of chemical precursors or non-protein-of-interest chemicals.

The use herein of the terms “polynucleotide”, “polynucleotide molecule”, “nucleic acid molecule”, nucleotide sequence” and the like is not intended to limit the present invention to polynucleotides, polynucleotide molecules, nucleic acid molecules and nucleotide sequences comprising DNA. Those of ordinary skill in the art will recognize that polynucleotides and oligonucleotides comprised of ribonucleotides and combinations of ribonucleotides and deoxyribonucleotides may also be employed in the methods disclosed herein. Thus, the present invention encompasses all polynucleotide constructs that can be employed in the methods of the present invention for transforming plants including, but not limited to, those comprised of deoxyribonucleotides, ribonucleotides and combinations thereof. Such deoxyribonucleotides and ribonucleotides include both naturally occurring forms and synthetic analogues. The polynucleotides of the invention also encompass all forms of nucleotide constructs including, but not limited to, single-stranded forms, double-stranded forms, hairpins, stem-and-loop structures and the like.

Fragments and variants of the disclosed polynucleotides and proteins encoded thereby are also encompassed by the present invention. By “fragment” is intended a portion of the polynucleotide or a portion of the amino acid sequence and hence of the protein encoded thereby. Fragments of a polynucleotide may encode protein fragments that retain biological activity of the native protein and hence histidine kinase activity. Alternatively, fragments of a polynucleotide that are useful as hybridization probes generally do not encode fragment proteins retaining biological activity. Thus, fragments of a nucleotide sequence may range from at least about 20 nucleotides, about 50 nucleotides, about 100 nucleotides and up to the full-length polynucleotide encoding the proteins of the invention.

A fragment of a histidine kinase polynucleotide that encodes a biologically active portion of a histidine kinase protein of the invention will encode at least 15, 25, 30, 50, 100, 150, 200, 250, 300, 350, 400, 450, 500, 550, 600, 650, 700, 800, 900, 1,000, 1,100 or 1,200 contiguous amino acids, or up to the total number of amino acids present in a full-length histidine kinase protein of the invention (for example, 966, 1007, 965, 918, 476, 968, 1059 and 1201 amino acids for SEQ ID NOS: 2, 5, 8, 14, 17, 23, 27 and 31, respectively). Fragments of a histidine kinase polynucleotide that are useful as hybridization probes or PCR primers generally need not encode a biologically active portion of a histidine kinase protein.

Thus, a fragment of a histidine kinase polynucleotide may encode a biologically active portion of a histidine kinase protein, or it may be a fragment that can be used as a hybridization probe or PCR primer using methods disclosed below. A biologically active portion of a histidine kinase protein can be prepared by isolating a portion of one of the histidine kinase polynucleotide of the invention, expressing the encoded portion of the histidine kinase protein (e.g., by recombinant expression in vitro), and assessing the activity of the encoded portion of the histidine kinase protein. Polynucleotides that are fragments of a histidine kinase nucleotide sequence comprise at least 16, 20, 50, 75, 100, 150, 200, 250, 300, 350, 400, 450, 500, 550, 600, 650, 700, 800, 900, 1,000, 1,100, 1,200, 1,300, 1,400, 1,500, 1,750, 2,000, 2,250, 2,500, 3,000, 3,500, 4,000 or 4500 nucleotides, or up to the number of nucleotides present in a full-length histidine kinase polynucleotide disclosed herein (for example, 2901, 2901, 3021, 3021, 2895, 2895, 3291, 2754, 1431, 1428, 3240, 3180, 3606 and 3606 nucleotides for SEQ ID NOS: 1, 3, 4, 6, 7, 9, 13, 15, 16, 18, 26, 28, 30 and 32, respectively).

“Variants” is intended to mean substantially similar sequences. For polynucleotides, a variant comprises a deletion and/or addition of one or more nucleotides at one or more sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the histidine kinase polypeptides of the invention. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotide, such as those generated, for example, by using site-directed mutagenesis histidine kinase protein of the invention. Generally, variants of a particular polynucleotide of the invention will have at least about 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Thus, for example, an isolated polynucleotide that encodes a polypeptide with a given percent sequence identity to the polypeptide of at least one sequence selected from the group consisting of SEQ ID NOS: 2, 5, 8, 14, 17, 23, 27 and 31, is disclosed. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 60%, 65%, 70%, 75%, 80%, 85%, 90%, 91%, 92%, 93%, 94%, 95%, 96%, 97%, 98%, 99% or more sequence identity.

Variant protein is intended to mean a protein derived from the native protein by deletion or addition of one or more amino acids at one or more sites in the native protein and/or substitution of one or more amino acids at one or more sites in the native protein. Variant proteins encompassed by the present invention are biologically active, that is they continue to possess the desired biological activity of the native protein, that is, histidine kinase activity as described herein. Such variants may result from, for example, genetic polymorphism or from human manipulation. Biologically active variants of a native histidine kinase protein of the invention will have at least about 60%, 65%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the invention may differ from that protein by as few as 1-15 amino acid residues, as few as 1-10, such as 6-10, as few as 5, as few as 4, 3, 2 or even 1 amino acid residue.

The proteins of the invention may be altered in various ways including amino acid substitutions, deletions, truncations, and insertions. Methods for such manipulations are generally known in the art. For example, amino acid sequence variants and fragments of the histidine kinase proteins can be prepared by mutations in the DNA. Methods for mutagenesis and polynucleotide alterations are well known in the art. See, for example, Kunkel, (1985) Proc. Natl. Acad. Sci. USA 82:488-492; Kunkel, et al., (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff, et al., (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

Thus, the genes and polynucleotides of the invention include both the naturally occurring sequences as well as mutant forms. Likewise, the proteins of the invention encompass both naturally occurring proteins as well as variations and modified forms thereof. Such variants will continue to possess the desired histidine kinase activity. Obviously, the mutations that will be made in the DNA encoding the variant must not place the sequence out of reading frame and optimally will not create complementary regions that could produce secondary mRNA structure. See, EP Patent Application Publication Number 75,444.

The deletions, insertions and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated by histidine kinase activity assays. See, for example, Posas, et al., (1996) Cell 86:865-875, herein incorporated by reference in its entirety. More recently, in vitro demonstrations for histidine kinase activity have been presented by Zhang, et al., 2004 (Plant Physiol. 136:2971-2981), herein incorporated by reference in its entirety. As the function of the yeast SLN1 protein as a histidine kinase is now unequivocally established, histidine kinase activity assays can also performed by complementation of the yeast sln1 mutant (Ueguchi, et al., (2001) Plant Cell Physiol. 42:231-235), herein incorporated by reference in its entirety.

Variant polynucleotides and proteins also encompass sequences and proteins derived from a mutagenic and recombinogenic procedure such as DNA shuffling. With such a procedure, one or more different histidine kinase coding sequences can be manipulated to create a new histidine kinase possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between a histidine kinase polynucleotide of the invention and other known histidine kinase polynucleotides to obtain a new gene coding for a protein with an improved property of interest, such as an increased Kₘ in the case of an enzyme. Strategies for such DNA shuffling are known in the art. See, for example, Stemmer, (1994) Proc. Natl. Acad. Sci. USA 91:10747-10751; Stemmer, (1994) Nature 370:389-391; Crameri, et al., (1997) Nature Biotech. 15:436-438; Moore, et al., (1997) J. Mol. Biol. 272:336-347; Zhang, et al., (1997) Proc. Natl. Acad. Sci. USA 94:4504-4509; Crameri, et al., (1998) Nature 391:288-291 and U.S. Pat. Nos. 5,605,793 and 5,837,458.

The polynucleotides of the invention can be used to isolate corresponding sequences from other organisms, particularly other plants, more particularly other monocots. In this manner, methods such as PCR, hybridization and the like can be used to identify such sequences based on their sequence homology to the sequences set forth herein. Sequences isolated based on their sequence identity to an entire histidine kinase sequences set forth herein or to variants and fragments thereof are encompassed by the present invention. Such sequences include sequences that are orthologs of the disclosed sequences. “Orthologs” is intended to mean genes derived from a common ancestral gene and which are found in different species as a result of speciation. Genes found in different species are considered orthologs when their nucleotide sequences and/or their encoded protein sequences share at least 60%, 70%, 75%, 80%, 85%, 90%, 91%, 92%, 93%, 94%, 95%, 96%, 97%, 98%, 99%, or greater sequence identity. Functions of orthologs are often highly conserved among species. Thus, isolated polynucleotides that encode for a histidine kinase protein and which hybridize under stringent conditions to at least one of the histidine kinase nucleotide sequences disclosed herein, or to variants or fragments thereof, are encompassed by the present invention.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook, et al., (1989) Molecular Cloning: A Laboratory Manual (2d ed., Cold Spring Harbor Laboratory Press, Plainview, N.Y.). See also, Innis, et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers and the like.

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the histidine kinase polynucleotides of the invention. Methods for preparation of probes for hybridization and for construction of cDNA and genomic libraries are generally known in the art and are disclosed in Sambrook, et al., (1989) Molecular Cloning: A Laboratory Manual (2d ed., Cold Spring Harbor Laboratory Press, Plainview, N.Y.).

For example, an entire histidine kinase polynucleotide disclosed herein, or one or more portions thereof, may be used as a probe capable of specifically hybridizing to corresponding histidine kinase polynucleotides and messenger RNAs. To achieve specific hybridization under a variety of conditions, such probes include sequences that are unique among histidine kinase polynucleotide sequences and are optimally at least about 10 nucleotides in length, and most optimally at least about 20 nucleotides in length. Such probes may be used to amplify corresponding histidine kinase polynucleotides from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook, et al., (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, optimally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. The duration of hybridization is generally less than about 24 hours, usually about 4 to about 12 hours. The duration of the wash will be at least a length of time sufficient to reach equilibrium. Typically, the duration of the wash will be about 1, 2, 5, 10, 15, 20, 30 or more minutes.

Specificity is typically the function of post-hybridization washes, the critical factors being the ionic strength and temperature of the final wash solution. For DNA-DNA hybrids, the Tₘ can be approximated from the equation of Meinkoth and Wahl, (1984) Anal. Biochem. 138:267-284: Tₘ=81.5° C.+16.6 (log M)+0.41 (% GC)-0.61 (% form)-500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is optimal to increase the SSC concentration so that a higher temperature can be used. An extensive guide to the hybridization of nucleic acids is found in Tijssen, (1993) Laboratory Techniques in Biochemistry and Molecular Biology—Hybridization with Nucleic Acid Probes, Part I, Chapter 2 (Elsevier, New York); and Ausubel, et al., eds. (1995) Current Protocols in Molecular Biology, Chapter 2 (Greene Publishing and Wiley-Interscience, New York). See, Sambrook, et al. (1989) Molecular Cloning: A Laboratory Manual (2d ed., Cold Spring Harbor Laboratory Press, Plainview, N.Y.).

The following terms are used to describe the sequence relationships between two or more polynucleotides or polypeptides: (a) “reference sequence”, (b) “comparison window”, (c) “sequence identity” and (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller, (1988) CABIOS 4:11-17; the local alignment algorithm of Smith, et al., (1981) Adv. Appl. Math. 2:482; the global alignment algorithm of Needleman and Wunsch, (1970) J. Mol. Biol. 48:443-453; the search-for-local alignment method of Pearson and Lipman, (1988) Proc. Natl. Acad. Sci. 85:2444-2448; the algorithm of Karlin and Altschul, (1990) Proc. Natl. Acad. Sci. USA 872264, modified as in Karlin and Altschul, (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.), including CLUSTALV and CLUSTALW; the ALIGN program (Version 2.0) and GAP, BESTFIT, BLAST, FASTA and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins, et al., (1988) Gene 73:237-244 (1988); Higgins, et al., (1989) CABIOS 5:151-153; Corpet, et al., (1988) Nucleic Acids Res. 16:10881-90; Huang, et al., (1992) CABIOS 8:155-65 and Pearson, et al., (1994) Meth. Mol. Biol. 24:307-331. The ALIGN program is based on the algorithm of Myers and Miller, (1988) supra. A PAM120 weight residue table, a gap length penalty of 12, and a gap penalty of 4 can be used with the ALIGN program when comparing amino acid sequences. The BLAST programs of Altschul, et al., (1990) J. Mol. Biol. 215:403 are based on the algorithm of Karlin and Altschul, (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul, et al., (1997) Nucleic Acids Res. 25:3389. Alternatively, PSI-BLAST (in BLAST 2.0) can be used to perform an iterated search that detects distant relationships between molecules. See, Altschul, et al., (1997) supra. When utilizing BLAST, Gapped BLAST, PSI-BLAST, the default parameters of the respective programs (e.g., BLASTN for nucleotide sequences, BLASTX for proteins) can be used. See the databases and programs maintained on-line by the United States National Center for Biotechnology Information of the National Institutes of Health. Alignment may also be performed manually by inspection.

GAP uses the algorithm of Needleman and Wunsch, (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG® Wisconsin Genetics Software Package (Accelrys, Inc., San Diego, Calif.) for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

Unless otherwise stated, sequence identity/similarity values provided herein refer to the value obtained using GAP Version 10 using the following parameters: % identity and % similarity for a nucleotide sequence using GAP Weight (gap creation penalty) of 50 and Length Weight (gap extension penalty) of 3, and the nwsgapdna.cmp scoring matrix; % identity and % similarity for an amino acid sequence using GAP Weight of 8 and Length Weight of 2, and the BLOSUM62 scoring matrix.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see, Henikoff and Henikoff, (1989) Proc. Natl. Acad. Sci. USA 89:10915).

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The histidine kinase polynucleotides of the invention can be provided in expression cassettes for expression in the plant of interest. The cassette will include 5′ and 3′ regulatory sequences operably linked to a histidine kinase polynucleotide of the invention. “Operably linked” is intended to mean a functional linkage between two or more elements. For example, an operable linkage between a polynucleotide of interest and a regulatory sequence (i.e., a promoter) is functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the histidine kinase polynucleotide to be under the transcriptional regulation of the regulatory regions. The expression cassette may additionally contain selectable marker genes.

The expression cassette will include in the 5′-3′ direction of transcription, a transcriptional and translational initiation region (i.e., a promoter), a histidine kinase polynucleotide of the invention, and a transcriptional and translational termination region (i.e., termination region) functional in plants. The regulatory regions (i.e., promoters, transcriptional regulatory regions, and translational termination regions) and/or the histidine kinase polynucleotide of the invention may be native/analogous to the host cell or to each other. Alternatively, the regulatory regions and/or the histidine kinase polynucleotide of the invention may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

While it may be optimal to express the sequences using heterologous promoters, the native promoter sequences may be used. Such constructs can change expression levels of histidine kinase in the plant or plant cell. Thus, the phenotype of the plant or plant cell can be altered.

The termination region may be native with the transcriptional initiation region, may be native with the operably linked histidine kinase polynucleotide of interest, may be native with the plant host, or may be derived from another source (i.e., foreign or heterologous) to the promoter, the histidine kinase polynucleotide of interest, the plant host, or any combination thereof. Convenient termination regions are available from the Ti-plasmid of A. tumefaciens, such as the octopine synthase and nopaline synthase termination regions. See also, Guerineau, et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903 and Joshi, et al., (1987) Nucleic Acids Res. 15:9627-9639.

Where appropriate, the polynucleotides may be optimized for increased expression in the transformed plant. That is, the polynucleotides can be synthesized using plant-preferred codons for improved expression. See, for example, Campbell and Gowri, (1990) Plant Physiol. 92:1-11 for a discussion of host-preferred codon usage. Methods are available in the art for synthesizing plant-preferred genes. See, for example, U.S. Pat. Nos. 5,380,831 and 5,436,391 and Murray, et al., (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein, et al., (1989) Proc. Natl. Acad. Sci. USA 86:6126-6130); potyvirus leaders, for example, TEV leader (Tobacco Etch Virus) (Gallie, et al., (1995) Gene 165(2):233-238), MDMV leader (Maize Dwarf Mosaic Virus) (Virology 154:9-20), and human immunoglobulin heavy-chain binding protein (BiP) (Macejak, et al., (1991) Nature 353:90-94); untranslated leader from the coat protein mRNA of alfalfa mosaic virus (AMV RNA 4) (Jobling, et al., (1987) Nature 325:622-625); tobacco mosaic virus leader (TMV) (Gallie, et al., (1989) in Molecular Biology of RNA, ed. Cech (Liss, New York), pp. 237-256); and maize chlorotic mottle virus leader (MCMV) (Lommel, et al., (1991) Virology 81:382-385). See also, Della-Cioppa, et al., (1987) Plant Physiol. 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention, including the native promoter of the polynucleotide sequence of interest. The promoters can be selected based on the desired outcome. The nucleic acids can be combined with constitutive, tissue-preferred, or other promoters for expression in plants.

Such constitutive promoters include, for example, the core promoter of the Rsyn7 promoter and other constitutive promoters disclosed in WO 99/43838 and U.S. Pat. No. 6,072,050; the core CaMV 35S promoter (Odell, et al., (1985) Nature 313:810-812); rice actin (McElroy, et al., (1990) Plant Cell 2:163-171); ubiquitin (Christensen, et al., (1989) Plant Mol. Biol. 12:619-632 and Christensen, et al., (1992) Plant Mol. Biol. 18:675-689); pEMU (Last, et al., (1991) Theor. Appl. Genet. 81:581-588); MAS (Velten, et al., (1984) EMBO J. 3:2723-2730); ALS promoter (U.S. Pat. No. 5,659,026), scp1 (WO 97/47756, U.S. Pat. No. 6,555,673), histone H2B promoter (U.S. Pat. No. 6,177,611) and the like. Other constitutive promoters include, for example, U.S. Pat. Nos. 5,608,149; 5,608,144; 5,604,121; 5,569,597; 5,466,785; 5,399,680; 5,268,463; 5,608,142 and 6,177,611.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize ln2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena, et al., (1991) Proc. Natl. Acad. Sci. USA 88:10421-10425 and McNellis, et al., (1998) Plant J. 14(2):247-257) and tetracycline-inducible and tetracycline-repressible promoters (see, for example, Gatz, et al., (1991) Mol. Gen. Genet. 227:229-237 and U.S. Pat. Nos. 5,814,618 and 5,789,156), herein incorporated by reference.

Tissue-preferred promoters can be utilized to target enhanced histidine kinase expression within a particular plant tissue. Tissue-preferred promoters include Yamamoto, et al., (1997) Plant J. 12(2):255-265; Kawamata, et al., (1997) Plant Cell Physiol. 38(7):792-803; Hansen, et al., (1997) Mol. Gen. Genet. 254(3):337-343; Russell, et al., (1997) Transgenic Res. 6(2):157-168; Rinehart, et al., (1996) Plant Physiol. 112(3):1331-1341; Van Camp, et al., (1996) Plant Physiol. 112(2):525-535; Canevascini, et al., (1996) Plant Physiol. 112(2):513-524; Yamamoto, et al., (1994) Plant Cell Physiol. 35(5):773-778; Lam, (1994) Results Probl. Cell Differ. 20:181-196; Orozco, et al., (1993) Plant Mol. Biol. 23(6):1129-1138; Matsuoka, et al., (1993) Proc Natl. Acad. Sci. USA 90(20):9586-9590 and Guevara-Garcia, et al., (1993) Plant J. 4(3):495-505. Such promoters can be modified, if necessary, for weak expression.

Leaf-preferred promoters are known in the art. See, for example, Yamamoto, et al., (1997) Plant J. 12(2):255-265; Kwon, et al., (1994) Plant Physiol. 105:357-67; Yamamoto, et al., (1994) Plant Cell Physiol. 35(5):773-778; Gotor, et al., (1993) Plant J. 3:509-18; Orozco, et al., (1993) Plant Mol. Biol. 23(6):1129-1138 and Matsuoka, et al., (1993) Proc. Natl. Acad. Sci. USA 90(20):9586-9590.

Root-preferred promoters are known and can be selected from the many available from the literature or isolated de novo from various compatible species. See, for example, Hire, et al., (1992) Plant Mol. Biol. 20(2):207-218 (soybean root-specific glutamine synthetase gene); Keller and Baumgartner, (1991) Plant Cell 3(10):1051-1061 (root-specific control element in the GRP 1.8 gene of French bean); Sanger, et al., (1990) Plant Mol. Biol. 14(3):433-443 (root-specific promoter of the mannopine synthase (MAS) gene of Agrobacterium tumefaciens) and Miao, et al., (1991) Plant Cell 3(1):11-22 (full-length cDNA clone encoding cytosolic glutamine synthetase (GS), which is expressed in roots and root nodules of soybean). See also, Bogusz, et al., (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi, (1991) describe their analysis of the promoters of the highly expressed rolC and rolD root-inducing genes of Agrobacterium rhizogenes (see, Plant Science (Limerick) 79(1):69-76). They concluded that enhancer and tissue-preferred DNA determinants are dissociated in those promoters. Teeri, et al., (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see, EMBO J. 8(2):343-350). The TR1′ gene, fused to nptII (neomycin phosphotransferase II) showed similar characteristics. Additional root-preferred promoters include the VfENOD-GRP3 gene promoter (Kuster, et al., (1995) Plant Mol. Biol. 29(4):759-772); and rolB promoter (Capana, et al., (1994) Plant Mol. Biol. 25(4):681-691. See also, U.S. Pat. Nos. 5,837,876; 5,750,386; 5,633,363; 5,459,252; 5,401,836; 5,110,732 and 5,023,179.

“Seed-preferred” promoters comprise those promoters active during seed development, including those active in the female reproductive tissue at or about the time of anthesis, and promoters of seed storage proteins. Promoters active during seed germination may also be of interest. See, Thompson, et al., (1989) BioEssays 10:108, herein incorporated by reference. Such seed-preferred promoters include, but are not limited to, Cim1 (cytokinin-induced message); cZ19B1 (maize 19 kDa zein); and milps (myo-inositol-1-phosphate synthase) (see, WO 00/11177 and U.S. Pat. No. 6,225,529; herein incorporated by reference). Gamma-zein is an endosperm-specific promoter. Globulin 1 (Glb-1) is a representative embryo-specific promoter. For dicots, seed-preferred promoters include, but are not limited to, bean β-phaseolin, napin, β-conglycinin, soybean lectin, cruciferin, and the like. For monocots, seed-preferred promoters include, but are not limited to, maize 15 kDa zein, 22 kDa zein, 27 kDa zein, gamma-zein, waxy, shrunken 1, shrunken 2, Globulin 1, etc. See also, WO 00/12733, where seed-preferred promoters from end1 and end2 genes are disclosed; herein incorporated by reference.

Additional embryo preferred promoters are disclosed in Sato, et al., (1996) Proc. Natl. Acad. Sci. 93:8117-8122; Nakase, et al., (1997) Plant J 12:235-46; and Postma-Haarsma, et al., (1999) Plant Mol. Biol. 39:257-71. Additional endosperm preferred promoters are disclosed in Albani, et al., (1984) EMBO 3:1405-15; Albani, et al., (1999) Theor. Appl. Gen. 98:1253-62; Albani, et al., (1993) Plant J. 4:343-55; Mena, et al., (1998) The Plant Journal 116:53-62 and Wu, et al., (1998) Plant Cell Physiology 39:885-889.

As stated above, promoters of interest include those active in meristem regions, such as developing inflorescence tissues, and promoters which drive expression at or about the time of anthesis or early kernel development. This may include, for example, the maize Zag2.1 promoter (GenBank X80206; see also, U.S. patent application Ser. No. 10/817,483); maize Zap promoter (also known as ZmMADS; U.S. patent application Ser. No. 10/387,937; WO 03/078590); maize ckx1-2 promoter (US Patent Application Publication Number 2002/0152500 A1; WO 02/0078438); maize eep1 promoter (U.S. patent application Ser. No. 10/817,483); maize end2 promoter (U.S. Pat. No. 6,528,704 and U.S. patent application Ser. No. 10/310,191); maize lec1 promoter (U.S. patent application Ser. No. 09/718,754); maize F3.7 promoter (Baszczynski, et al., (1997) Maydica 42:189-201); maize tb1 promoter (Hubbarda, et al., (2002) Genetics 162:1927-1935); maize eep2 promoter (U.S. patent application Ser. No. 10/817,483); maize thioredoxinH promoter (U.S. Provisional Patent Application Ser. No. 60/514,123); maize Zm40 promoter (U.S. Pat. No. 6,403,862 and WO 01/2178); maize mLIP15 promoter (U.S. Pat. No. 6,479,734); maize ESR promoter (U.S. patent application Ser. No. 10/786,679); maize PCNA2 promoter (U.S. patent application Ser. No. 10/388,359); maize cytokinin oxidase promoters (U.S. Provisional Patent Application Ser. No. 60/559,252).

Shoot-preferred promoters include, shoot meristem-preferred promoters such as promoters disclosed in Weigal, et al., (1992) Cell 69:843-859; Accession Number AJ131822; Accession Number Z71981; Accession Number AF049870; the ZAP promoter (U.S. patent application Ser. No. 10/387,937); maize tbl promoter (Wang, et al., (1999) Nature 398:236-239) and shoot-preferred promoters disclosed in McAvoy, et al., (2003) Acta Hort. (ISHS) 625:379-385.

Dividing cell or meristematic tissue-preferred promoters have been disclosed in Ito, et al., (1994) Plant Mol. Biol. 24:863-878; Regad, et al., (1995) Mo. Gen. Genet. 248:703-711; Shaul, et al., (1996) Proc. Natl. Acad. Sci. 93:4868-4872; Ito, et al., (1997) Plant J. 11:983-992 and Trehin, et al., (1997) Plant Mol. Biol. 35:667-672; Zag1 (Schmidt, et al., (1993) The Plant Cell 5:729-37 and Zag2 from maize (Theissen, et al., (1995) Gene 156:155-166), Genbank Accession Number X80206, all of which are herein incorporated by reference.

Inflorescence-preferred promoters include the promoter of chalcone synthase (Van der Meer, et al., (1990) Plant Mol. Biol. 15:95-109), LAT52 (Twell, et al., (1989) Mol. Gen. Genet. 217:240-245), pollen specific genes (Albani, et al., (1990) Plant Mol. Biol. 15:605), Zm13 (Buerrero, et al., (1993) Mol. Gen. Genet. 224:161-168), maize pollen-specific gene (Hamilton, et al., (1992) Plant Mol. Biol. 18:211-218), sunflower pollen expressed gene (Baltz, et al., (1992) The Plant Journal 2:713-721) and B. napus pollen specific genes (Arnoldo, et al., (1992) J. Cell. Biochem, Abstract No. Y101204).

Stress inducible promoters include salt/water stress-inducible promoters such as P5CS (Zang, et al., (1997) Plant Sciences 129:81-89); cold-inducible promoters, such as, cor15a (Hajela, et al., (1990) Plant Physiol. 93:1246-1252), cor15b (Wilhelm, et al., (1993) Plant Mol Biol 23:1073-1077), wsc120 (Ouellet, et al., (1998) FEBS Lett. 423-324-328), ci7 (Kirch, et al., (1997) Plant Mol. Biol. 33:897-909), ci21A (Schneider, et al., (1997) Plant Physiol. 113:335-45); drought-inducible promoters, such as, Trg-31 (Chaudhary, et al., (1996) Plant Mol. Biol. 30:1247-57); osmotic inducible promoters, such as, Rab17 (Vilardell, et al., (1991) Plant Mol. Biol. 17:985-93) and osmotin (Raghothama, et al., (1993) Plant Mol Biol 23:1117-28); and, heat inducible promoters, such as, heat shock proteins (Barros, et al., (1992) Plant Mol. 19:665-75; Marrs, et al., (1993) Dev. Genet. 14:27-41), and smHSP (Waters, et al., (1996) J. Experimental Botany 47:325-338). Other stress-inducible promoters include rip2 (U.S. Pat. No. 5,332,808 and US Patent Application Publication Number 2003/0217393) and rd29a (Yamaguchi-Shinozaki, et al., (1993) Mol. Gen. Genetics 236:331-340). Also of interest are senescence-preferred promoters, such as SEE1 (NCBI AJ494982) and SAG12 (NCBI U37336).

Stress-insensitive promoters can also be used in the methods of the invention. By “stress-insensitive” is intended that the expression level of a sequence operably linked to the promoter is not altered or only minimally altered under stress conditions such as drought or heat.

Nitrogen-responsive promoters can also be used in the methods of the invention. Such promoters include, but are not limited to, the 22 kDa Zein promoter (Spena, et al., (1982) EMBO J. 1:1589-1594 and Muller, et al., (1995) J. Plant Physiol 145:606-613); the 19 kDa zein promoter (Pedersen, et al., (1982) Cell 29:1019-1025); the 14 kDa zein promoter (Pedersen, et al., (1986) J. Biol. Chem. 261:6279-6284), the b-32 promoter (Lohmer, et al., (1991) EMBO J. 10:617-624); and the nitrite reductase (NiR) promoter (Rastogi, et al., (1997) Plant Mol. Biol. 34(3):465-76 and Sander, et al., (1995) Plant Mol. Biol. 27(1):165-77). For a review of consensus sequences found in nitrogen-induced promoters, see for example, Muller, et al., (1997) The Plant Journal 12:281-291.

Where low level expression is desired, weak promoters will be used. Generally, by “weak promoter” is intended a promoter that drives expression of a coding sequence at a low level. By low level is intended at levels of about 1/1000 transcripts to about 1/100,000 transcripts to about 1/500,000 transcripts. Alternatively, it is recognized that weak promoters also encompasses promoters that are expressed in only a few cells and not in others to give a total low level of expression. Where a promoter is expressed at unacceptably high levels, portions of the promoter sequence can be deleted or modified to decrease expression levels.

Such weak constitutive promoters include, for example, the core promoter of the Rsyn7 promoter (WO 99/43838 and U.S. Pat. No. 6,072,050), the core 35S CaMV promoter, and the like. Other constitutive promoters include, for example, U.S. Pat. Nos. 5,608,149; 5,608,144; 5,604,121; 5,569,597; 5,466,785; 5,399,680; 5,268,463 and 5,608,142. See also, U.S. Pat. No. 6,177,611, herein incorporated by reference.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su, et al., (2004) Biotechnol Bioeng 85:610-9 and Fetter, et al., (2004) Plant Cell 16:215-28), cyan fluorescent protein (CYP) (Bolte, et al., (2004) J. Cell Science 117:943-54 and Kato, et al., (2002) Plant Physiol 129:913-42) and yellow fluorescent protein (PhiYFP™ from Evrogen, see, Bolte, et al., (2004) J. Cell Science 117:943-54). For additional selectable markers, see generally, Yarranton, (1992) Curr. Opin. Biotech. 3:506-511; Christopherson, et al., (1992) Proc. Natl. Acad. Sci. USA 89:6314-6318; Yao, et al., (1992) Cell 71:63-72; Reznikoff, (1992) Mol. Microbiol. 6:2419-2422; Barkley, et al., (1980) in The Operon, pp. 177-220; Hu, et al., (1987) Cell 48:555-566; Brown, et al., (1987) Cell 49:603-612; Figge, et al., (1988) Cell 52:713-722; Deuschle, et al., (1989) Proc. Natl. Acad. Aci. USA 86:5400-5404; Fuerst, et al., (1989) Proc. Natl. Acad. Sci. USA 86:2549-2553; Deuschle, et al., (1990) Science 248:480-483; Gossen, (1993) Ph.D. Thesis, University of Heidelberg; Reines, et al., (1993) Proc. Natl. Acad. Sci. USA 90:1917-1921; Labow, et al., (1990) Mol. Cell. Biol. 10:3343-3356; Zambretti, et al., (1992) Proc. Natl. Acad. Sci. USA 89:3952-3956; Baim, et al., (1991) Proc. Natl. Acad. Sci. USA 88:5072-5076; Wyborski, et al., (1991) Nucleic Acids Res. 19:4647-4653; Hillenand-Wissman, (1989) Topics Mol. Struc. Biol. 10:143-162; Degenkolb, et al., (1991) Antimicrob. Agents Chemother. 35:1591-1595; Kleinschnidt, et al., (1988) Biochemistry 27:1094-1104; Bonin, (1993) Ph.D. Thesis, University of Heidelberg; Gossen, et al., (1992) Proc. Natl. Acad. Sci. USA 89:5547-5551; Oliva, et al., (1992) Antimicrob. Agents Chemother. 36:913-919; Hlavka, et al., (1985) Handbook of Experimental Pharmacology, Vol. 78 (Springer-Verlag, Berlin); Gill, et al., (1988) Nature 334:721-724. Such disclosures are herein incorporated by reference.

The above list of selectable marker genes is not meant to be limiting. Any selectable marker gene can be used in the present invention.

In one example, the polynucleotide of interest is targeted to the chloroplast for expression. In this manner, where the polynucleotide of interest is not directly inserted into the chloroplast, the expression cassette will additionally contain a nucleic acid encoding a transit peptide to direct the gene product of interest to the chloroplasts. Such transit peptides are known in the art. See, for example, Von Heijne, et al., (1991) Plant Mol. Biol. Rep. 9:104-126; Clark, et al., (1989) J. Biol. Chem. 264:17544-17550; Della-Cioppa, et al., (1987) Plant Physiol. 84:965-968; Romer, et al., (1993) Biochem. Biophys. Res. Commun. 196:1414-1421 and Shah, et al., (1986) Science 233:478-481.

Chloroplast targeting sequences are known in the art and include the chloroplast small subunit of ribulose-1,5-bisphosphate carboxylase (Rubisco) (de Castro Silva Filho, et al., (1996) Plant Mol. Biol. 30:769-780; Schnell, et al., (1991) J. Biol. Chem. 266(5):3335-3342); 5-(enolpyruvyl)shikimate-3-phosphate synthase (EPSPS) (Archer, et al., (1990) J. Bioenerg. Biomemb. 22(6):789-810); tryptophan synthase (Zhao, et al., (1995) J. Biol. Chem. 270(11):6081-6087); plastocyanin (Lawrence, et al., (1997) J. Biol. Chem. 272(33):20357-20363); chorismate synthase (Schmidt, et al., (1993) J. Biol. Chem. 268(36):27447-27457); and the light harvesting chlorophyll a/b binding protein (LHBP) (Lamppa, et al., (1988) J. Biol. Chem. 263:14996-14999). See also, Von Heijne, et al., (1991) Plant Mol. Biol. Rep. 9:104-126; Clark, et al., (1989) J. Biol. Chem. 264:17544-17550; Della-Cioppa, et al., (1987) Plant Physiol. 84:965-968; Romer, et al., (1993) Biochem. Biophys. Res. Commun. 196:1414-1421 and Shah, et al., (1986) Science 233:478-481.

Methods for transformation of chloroplasts are known in the art. See, for example, Svab, et al., (1990) Proc. Natl. Acad. Sci. USA 87:8526-8530; Svab and Maliga, (1993) Proc. Natl. Acad. Sci. USA 90:913-917; Svab and Maliga, (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride, et al., (1994) Proc. Natl. Acad. Sci. USA 91:7301-7305.

The polynucleotides of interest to be targeted to the chloroplast may be optimized for expression in the chloroplast to account for differences in codon usage between the plant nucleus and this organelle. In this manner, the polynucleotide of interest may be synthesized using chloroplast-preferred codons. See, for example, U.S. Pat. No. 5,380,831, herein incorporated by reference.

The methods of the invention involve introducing a polypeptide or polynucleotide into a plant. “Introducing” is intended to mean presenting to the plant the polynucleotide or polypeptide in such a manner that the sequence gains access to the interior of a cell of the plant. The methods of the invention do not depend on a particular method for introducing a sequence into a plant, only that the polynucleotide or polypeptides gains access to the interior of at least one cell of the plant. Methods for introducing polynucleotide or polypeptides into plants are known in the art including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway, et al., (1986) Biotechniques 4:320-334), electroporation (Riggs, et al., (1986) Proc. Natl. Acad. Sci. USA 83:5602-5606, Agrobacterium-mediated transformation (Townsend, et al., U.S. Pat. No. 5,563,055; Zhao, et al., U.S. Pat. No. 5,981,840), direct gene transfer (Paszkowski, et al., (1984) EMBO J. 3:2717-2722), and ballistic particle acceleration (see, for example, Sanford, et al., U.S. Pat. No. 4,945,050; Tomes, et al., U.S. Pat. No. 5,879,918; Tomes, et al., U.S. Pat. No. 5,886,244; Bidney, et al., U.S. Pat. No. 5,932,782; Tomes, et al., (1995) “Direct DNA Transfer into Intact Plant Cells via Microprojectile Bombardment,” in Plant Cell, Tissue, and Organ Culture: Fundamental Methods, ed. Gamborg and Phillips, (Springer-Verlag, Berlin); McCabe, et al., (1988) Biotechnology 6:923-926) and Lec1 transformation (WO 00/28058). Also see, Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P:175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); Tomes, U.S. Pat. No. 5,240,855; Buising, et al., U.S. Pat. Nos. 5,322,783 and 5,324,646; Tomes, et al., (1995) “Direct DNA Transfer into Intact Plant Cells via Microprojectile Bombardment,” in Plant Cell, Tissue, and Organ Culture: Fundamental Methods, ed. Gamborg, (Springer-Verlag, Berlin) (maize); Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; Bowen, et al., U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, New York), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotechnology 14:745-750 (maize via Agrobacterium tumefaciens); all of which are herein incorporated by reference.

In specific examples, the histidine kinase sequences of the invention can be provided to a plant using a variety of transient transformation methods. Such transient transformation methods include, but are not limited to, the introduction of histidine kinase protein or variants and fragments thereof directly into the plant or the introduction of a histidine kinase transcript into the plant. Such methods include, for example, microinjection or particle bombardment. See, for example, Crossway, et al., (1986) Mol. Gen. Genet. 202:179-185; Nomura, et al., (1986) Plant Sci. 44:53-58; Hepler, et al., (1994) Proc. Natl. Acad. Sci. 91:2176-2180 and Hush, et al., (1994) The Journal of Cell Science 107:775-784, all of which are herein incorporated by reference. Alternatively, the histidine kinase polynucleotide can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which its released to become integrated into the genome is greatly reduced. Such methods include the use particles coated with polyethylimine (PEI; Sigma #P3143).

In other examples, the polynucleotide of the invention may be introduced into plants by contacting plants with a virus or viral nucleic acids. Generally, such methods involve incorporating a nucleotide construct of the invention within a viral DNA or RNA molecule. It is recognized that the a histidine kinase of the invention may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931 and Porta, et al., (1996) Molecular Biotechnology 5:209-221; herein incorporated by reference.

Methods are known in the art for the targeted insertion of a polynucleotide at a specific location in the plant genome. In one example, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide of the invention can be contained in transfer cassette flanked by two non-identical recombination sites. The transfer cassette is introduced into a plant have stably incorporated into its genome a target site which is flanked by two non-identical recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick, et al., (1986) Plant Cell Reports 5:81-84. These plants may then be grown, and either pollinated with the same transformed strain or different strains, and the resulting progeny having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide of the invention, for example, an expression cassette of the invention, stably incorporated into their genome.

Pedigree breeding starts with the crossing of two genotypes, such as an elite line of interest and one other inbred line having one or more desirable characteristics (i.e., having stably incorporated a polynucleotide of the invention, having a modulated activity and/or level of the polypeptide of the invention, etc) which complements the elite line of interest. If the two original parents do not provide all the desired characteristics, other sources can be included in the breeding population. In the pedigree method, superior plants are selfed and selected in successive filial generations. In the succeeding filial generations the heterozygous condition gives way to homogeneous lines as a result of self-pollination and selection. Typically in the pedigree method of breeding, five or more successive filial generations of selfing and selection is practiced: F₁→F₂; F₂→F₃; F₃→F₄; F₄→F₅, etc. After a sufficient amount of inbreeding, successive filial generations will serve to increase seed of the developed inbred. Preferably, the inbred line comprises homozygous alleles at about 95% or more of its loci.

In addition to being used to create a backcross conversion, backcrossing can also be used in combination with pedigree breeding to modify an elite line of interest and a hybrid that is made using the modified elite line. As discussed previously, backcrossing can be used to transfer one or more specifically desirable traits from one line, the donor parent, to an inbred called the recurrent parent, which has overall good agronomic characteristics yet lacks that desirable trait or traits. However, the same procedure can be used to move the progeny toward the genotype of the recurrent parent but at the same time retain many components of the non-recurrent parent by stopping the backcrossing at an early stage and proceeding with selfing and selection. For example, an F₁, such as a commercial hybrid, is created. This commercial hybrid may be backcrossed to one of its parent lines to create a BC₁ or BC₂. Progeny are selfed and selected so that the newly developed inbred has many of the attributes of the recurrent parent and yet several of the desired attributes of the non-recurrent parent. This approach leverages the value and strengths of the recurrent parent for use in new hybrids and breeding.

Therefore, an example of this invention is a method of making a backcross conversion of maize inbred line of interest, comprising the steps of crossing a plant of maize inbred line of interest with a donor plant comprising a mutant gene or transgene conferring a desired trait (e.g., increased expression of a histidine kinase of the invention), selecting an F₁ progeny plant comprising the mutant gene or transgene conferring the desired trait, and backcrossing the selected F₁ progeny plant to the plant of maize inbred line of interest. This method may further comprise the step of obtaining a molecular marker profile of maize inbred line of interest and using the molecular marker profile to select for a progeny plant with the desired trait and the molecular marker profile of the inbred line of interest. In the same manner, this method may be used to produce an F₁ hybrid seed by adding a final step of crossing the desired trait conversion of maize inbred line of interest with a different maize plant to make F₁ hybrid maize seed comprising a mutant gene or transgene conferring the desired trait.

Recurrent selection is a method used in a plant breeding program to improve a population of plants. The method entails individual plants cross pollinating with each other to form progeny. The progeny are grown and the superior progeny selected by any number of selection methods, which include individual plant, half-sib progeny, full-sib progeny, selfed progeny and topcrossing. The selected progeny are cross-pollinated with each other to form progeny for another population. This population is planted and again superior plants are selected to cross pollinate with each other. Recurrent selection is a cyclical process and therefore can be repeated as many times as desired. The objective of recurrent selection is to improve the traits of a population. The improved population can then be used as a source of breeding material to obtain inbred lines to be used in hybrids or used as parents for a synthetic cultivar. A synthetic cultivar is the resultant progeny formed by the intercrossing of several selected inbreds.

Mass selection is a useful technique when used in conjunction with molecular marker enhanced selection. In mass selection seeds from individuals are selected based on phenotype and/or genotype. These selected seeds are then bulked and used to grow the next generation. Bulk selection requires growing a population of plants in a bulk plot, allowing the plants to self-pollinate, harvesting the seed in bulk and then using a sample of the seed harvested in bulk to plant the next generation. Instead of self pollination, directed pollination could be used as part of the breeding program.

As used herein, the term plant includes plant cells, plant protoplasts, plant cell tissue cultures from which a plant can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers and the like. Grain is intended to mean the mature seed produced by commercial growers for purposes other than growing or reproducing the species. Progeny, variants and mutants of the regenerated plants are also included within the scope of the invention, provided that these parts comprise the introduced polynucleotides.

The present invention may be used for transformation of any plant species, including monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima) and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific examples, plants of the present invention are crop plants (for example, corn, alfalfa, sunflower, Brassica, soybean, cotton, safflower, peanut, sorghum, wheat, millet, tobacco, etc.). In other examples, corn and soybean plants are optimal, and in yet other examples corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

The histidine kinases of the invention can be produced in any host cell of interest. The polynucleotides of the invention can be used to express the histidine kinases of the invention in non-human host cells, including, but not limited to, plant cells, algal cells, bacterial cells, animal cells and fungal cells. Such fungal cells include, for example, yeast cells. For expression in a host cell of interest, a polynucleotide of the invention is operably linked to a promoter that drives expression in the host cell. The invention does not depend on a particular promoter or method for transforming a host cell with a polynucleotide construct. Any promoter and/or any method for transforming a host cell of interest can be used in the methods of the present invention. Thus, the present invention further provides non-human host cells transformed with at least one polynucleotide of the invention and methods for making such transformed host cells.

A method for modulating the concentration and/or activity of the polypeptide of the present invention in a plant is provided. In general, concentration and/or activity is increased or decreased by at least 1%, 5%, 10%, 20%, 30%, 40%, 50%, 60%, 70%, 80% or 90% relative to a native control plant, plant part or cell which did not have the sequence of the invention introduced. Modulation in the present invention may occur during and/or subsequent to growth of the plant to the desired stage of development. In specific examples, the polypeptides of the present invention are modulated in monocots, particularly maize.

The expression level of the histidine kinase polypeptide may be measured directly, for example, by assaying for the level of the histidine kinase polypeptide in the plant, or indirectly, for example, by measuring the histidine kinase activity in the plant, or by monitoring the plant phenotype. Methods for determining histidine kinase activity are described elsewhere herein.

In specific examples, the polypeptide or the polynucleotide of the invention is introduced into the plant cell. Subsequently, a plant cell having the introduced sequence of the invention is selected using methods known to those of skill in the art such as, but not limited to, Southern blot analysis, DNA sequencing, PCR analysis, or phenotypic analysis. A plant or plant part altered or modified by the foregoing embodiments is grown under plant forming conditions for a time sufficient to modulate the concentration and/or activity of polypeptides of the present invention in the plant. Plant forming conditions are well known in the art and discussed briefly elsewhere herein.

It is also recognized that the level and/or activity of the polypeptide may be modulated by employing a polynucleotide that is not capable of directing, in a transformed plant, the expression of a protein or an RNA. For example, the polynucleotides of the invention may be used to design polynucleotide constructs that can be employed in methods for altering or mutating a genomic nucleotide sequence in an organism. Such polynucleotide constructs include, but are not limited to, RNA:DNA vectors, RNA:DNA mutational vectors, RNA:DNA repair vectors, mixed-duplex oligonucleotides, self-complementary RNA:DNA oligonucleotides and recombinogenic oligonucleobases. Such nucleotide constructs and methods of use are known in the art. See, U.S. Pat. Nos. 5,565,350; 5,731,181; 5,756,325; 5,760,012; 5,795,972 and 5,871,984; all of which are herein incorporated by reference. See also, WO 98/49350, WO 99/07865, WO 99/25821, and Beetham, et al., (1999) Proc. Natl. Acad. Sci. USA 96:8774-8778; herein incorporated by reference.

It is therefore recognized that methods of the present invention do not depend on the incorporation of the entire polynucleotide into the genome, only that the plant or cell thereof is altered as a result of the introduction of the polynucleotide into a cell. In one example of the invention, the genome may be altered following the introduction of the polynucleotide into a cell. For example, the polynucleotide, or any part thereof, may incorporate into the genome of the plant. Alterations to the genome of the present invention include, but are not limited to, additions, deletions, and substitutions of nucleotides into the genome. While the methods of the present invention do not depend on additions, deletions, and substitutions of any particular number of nucleotides, it is recognized that such additions, deletions, or substitutions comprises at least one nucleotide.

In one example, the activity and/or level of the histidine kinase polypeptide of the invention is increased. An increase in the level and/or activity of the histidine kinase polypeptide of the invention can be achieved by providing to the plant a histidine kinase polypeptide. As discussed elsewhere herein, many methods are known the art for providing a polypeptide to a plant including, but not limited to, direct introduction of the polypeptide into the plant, and introducing into the plant (transiently or stably) a polynucleotide construct encoding a polypeptide having histidine kinase activity. It is also recognized that the methods of the invention may employ a polynucleotide that is not capable of directing, in the transformed plant, the expression of a protein or an RNA. Thus, the level and/or activity of a histidine kinase polypeptide may be increased by altering the gene encoding the histidine kinase polypeptide or its promoter. See, e.g., Kmiec, U.S. Pat. No. 5,565,350; Zarling, et al., PCT/US93/03868. Therefore mutagenized plants that carry mutations in histidine kinase genes, where the mutations increase expression of the histidine kinase gene or increase the histidine kinase activity of the encoded histidine kinase polypeptide are provided.

In other examples, the activity and/or level of the histidine kinase polypeptide of the invention is reduced or eliminated by introducing into a plant a polynucleotide that inhibits the level or activity of the histidine kinase polypeptide of the invention. The polynucleotide may inhibit the expression of histidine kinase directly, by preventing translation of the histidine kinase messenger RNA, or indirectly, by inhibiting the transcription or translation of a histidine kinase gene encoding a histidine kinase protein. Methods for inhibiting or eliminating the expression of a gene in a plant are well known in the art, and any such method may be used in the present invention to inhibit the expression of histidine kinase in a plant. In other examples of the invention, the activity of a histidine kinase polypeptide is reduced or eliminated by transforming a plant cell with a sequence encoding a polypeptide that inhibits the activity of the histidine kinase polypeptide. In other examples, the activity of a histidine kinase polypeptide may be reduced or eliminated by disrupting the gene encoding the histidine kinase polypeptide. The invention encompasses mutagenized plants that carry mutations in histidine kinase genes, where the mutations reduce expression of the histidine kinase gene or inhibit the histidine kinase activity of the encoded histidine kinase polypeptide.

Reduction of the activity of specific genes (also known as gene silencing or gene suppression) is desirable for several aspects of genetic engineering in plants. Many techniques for gene silencing are well known to one of skill in the art, including, but not limited to, antisense technology (see, e.g., Sheehy, et al., (1988) Proc. Natl. Acad. Sci. USA 85:8805-8809 and U.S. Pat. Nos. 5,107,065; 5,453,566 and 5,759,829); cosuppression (e.g., Taylor, (1997) Plant Cell 9:1245; Jorgensen, (1990) Trends Biotech. 8(12):340-344; Flavell, (1994) Proc. Natl. Acad. Sci. USA 91:3490-3496; Finnegan, et al., (1994) Bio/Technology 12:883-888 and Neuhuber, et al., (1994) Mol. Gen. Genet. 244:230-241); RNA interference (Napoli, et al., (1990) Plant Cell 2:279-289; U.S. Pat. No. 5,034,323; Sharp, (1999) Genes Dev. 13:139-141; Zamore, et al., (2000) Cell 101:25-33; and Montgomery, et al., (1998) Proc. Natl. Acad. Sci. USA 95:15502-15507), virus-induced gene silencing (Burton, et al., (2000) Plant Cell 12:691-705 and Baulcombe, (1999) Curr. Op. Plant Bio. 2:109-113); target-RNA-specific ribozymes (Haseloff, et al., (1988) Nature 334:585-591); hairpin structures (Mette, et al., (2002) EMBO J. 19:5194-5201; Smith, et al., (2000) Nature 407:319-320; WO 99/53050; WO 02/00904; WO 98/53083; Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini, et al., BMC Biotechnology 3:7, US Patent Application Publication Number 2003/0175965; Panstruga, et al., (2003) Mol. Biol. Rep. 30:135-140; Wesley, et al., (2001) Plant J. 27:581-590; Wang and Waterhouse, (2001) Curr. Opin. Plant Biol. 5:146-150; US Patent Application Publication Number 2003/0180945 and WO 02/00904, all of which are herein incorporated by reference); ribozymes (Steinecke, et al., (1992) EMBO J. 11:1525; and Perriman, et al., (1993) Antisense Res. Dev. 3:253); oligonucleotide-mediated targeted modification (e.g., WO 03/076574 and WO 99/25853); Zn-finger targeted molecules (e.g., WO 01/52620; WO 03/048345 and WO 00/42219); transposon tagging (Maes, et al., (1999) Trends Plant Sci. 4:90-96; Dharmapuri and Sonti, (1999) FEMS Microbiol. Lett. 179:53-59; Meissner, et al., (2000) Plant J. 22:265-274; Phogat, et al., (2000) J. Biosci. 25:57-63; Walbot, (2000) Curr. Opin. Plant Biol. 2:103-107; Gai, et al., (2000) Nucleic Acids Res. 28:94-96; Fitzmaurice, et al., (1999) Genetics 153:1919-1928; Bensen, et al., (1995) Plant Cell 7:75-84; Mena, et al., (1996) Science 274:1537-1540; and U.S. Pat. No. 5,962,764); each of which is herein incorporated by reference; and other methods or combinations of the above methods known to those of skill in the art.

The polynucleotides of the present invention may be used in the sense orientation to suppress the expression of endogenous genes in plants. Methods for suppressing gene expression in plants using polynucleotides in the sense orientation are known in the art. The methods generally involve transforming plants with a DNA construct comprising a promoter that drives expression in a plant operably linked to at least a portion of a polynucleotide that corresponds to the transcript of the endogenous gene. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. Overexpression of the RNA molecule can result in reduced expression of the native gene. Accordingly, multiple plant lines transformed with the cosuppression expression cassette are screened to identify those that show the greatest inhibition of histidine kinase expression. See, U.S. Pat. Nos. 5,283,184 and 5,034,323; hereby incorporated by reference. Thus, many methods may be used to reduce or eliminate the activity of a histidine kinase polypeptide. More than one method may be used to reduce the activity of a single histidine kinase polypeptide. In addition, combinations of methods may be employed to reduce or eliminate the activity of the histidine kinase polypeptides.

In some examples of the present invention, a maize plant cell is transformed with an expression cassette that is capable of expressing a polynucleotide that inhibits the expression of histidine kinase. The term “expression” as used herein refers to the biosynthesis of a gene product, including the transcription and/or translation of said gene product. For example, for the purposes of the present invention, an expression cassette capable of expressing a polynucleotide that inhibits the expression of at least one maize histidine kinase is an expression cassette capable of producing an RNA molecule that inhibits the transcription and/or translation of at least one maize histidine kinase. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

The polynucleotide used for cosuppression may correspond to all or part of the sequence encoding the histidine kinase, all or part of the 5′ and/or 3′ untranslated region of a histidine kinase transcript, or all or part of both the coding sequence and the untranslated regions of a transcript encoding histidine kinase. In some embodiments where the polynucleotide comprises all or part of the coding region for the histidine kinase, the expression cassette is designed to eliminate the start codon of the polynucleotide so that no protein product will be transcribed.

Cosuppression may be used to inhibit the expression of plant genes to produce plants having undetectable protein levels for the proteins encoded by these genes. See, for example, Broin, et al., (2002) Plant Cell 14:1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell, et al., (1994) Proc. Natl. Acad. Sci. USA 91:3490-3496; Jorgensen, et al., (1996) Plant Mol. Biol. 31:957-973; Johansen and Carrington, (2001) Plant Physiol. 126:930-938; Broin, et al., (2002) Plant Cell 14:1417-1432; Stoutjesdijk, et al., (2002) Plant Physiol. 129:1723-1731; Yu, et al., (2003) Phytochemistry 63:753-763 and U.S. Pat. Nos. 5,034,323, 5,283,184 and 5,942,657; each of which is herein incorporated by reference. The efficiency of cosuppression may be increased by including a poly-dT region in the expression cassette at a position 3′ to the sense sequence and 5′ of the polyadenylation signal. See, US Patent Application Publication Number 2002/0048814, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,184 and 5,034,323; herein incorporated by reference.

In some examples of the invention, inhibition of the expression of the histidine kinase may be obtained by antisense suppression. For antisense suppression, the expression cassette is designed to express an RNA molecule complementary to all or part of a messenger RNA encoding the histidine kinase. Overexpression of the antisense RNA molecule can result in reduced expression of the native gene. Accordingly, multiple plant lines transformed with the antisense suppression expression cassette are screened to identify those that show the greatest inhibition of histidine kinase expression.

The polynucleotide for use in antisense suppression is designed to hybridize with the corresponding mRNA and may comprise all or part of the complement of the sequence encoding the histidine kinase, all or part of the complement of the 5′ and/or 3′ untranslated region of the histidine kinase transcript, or all or part of the complement of both the coding sequence and the untranslated regions of a transcript encoding the histidine kinase. In addition, the antisense polynucleotide may be fully complementary (i.e., 100% identical to the complement of the target sequence) or partially complementary (i.e., less than 100% identical to the complement of the target sequence) to the target sequence. Antisense suppression may be used to inhibit the expression of multiple proteins in the same plant. See, for example, U.S. Pat. No. 5,942,657. Furthermore, portions of the antisense nucleotides may be used to disrupt the expression of the target gene. Generally, sequences of at least 50 nucleotides, 100 nucleotides, 200 nucleotides, 300, 400, 450, 500, 550 or greater may be used. Methods for using antisense suppression to inhibit the expression of endogenous genes in plants are described, for example, in Liu, et al., (2002) Plant Physiol. 129:1732-1743 and U.S. Pat. Nos. 5,759,829 and 5,942,657, each of which is herein incorporated by reference. Efficiency of antisense suppression may be increased by including a poly-dT region in the expression cassette at a position 3′ to the antisense sequence and 5′ of the polyadenylation signal. See, US Patent Application Publication Number 2002/0048814, herein incorporated by reference.

In some examples of the invention, inhibition of the expression of a histidine kinase may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the greatest inhibition of histidine kinase expression. Methods for using dsRNA interference to inhibit the expression of endogenous plant genes are described in Waterhouse, et al., (1998) Proc. Natl. Acad. Sci. USA 95:13959-13964, Liu, et al., (2002) Plant Physiol. 129:1732-1743 and WO 99/49029, WO 99/53050, WO 99/61631 and WO 00/49035; each of which is herein incorporated by reference.

In some examples of the invention, inhibition of the expression of one or more histidine kinases may be obtained by hairpin RNA (hpRNA) interference or intron-containing hairpin RNA (ihpRNA) interference. These methods are highly efficient at inhibiting the expression of endogenous genes. See, Waterhouse and Helliwell, (2003) Nat. Rev. Genet. 4:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited, and an antisense sequence that is fully or partially complementary to the sense sequence. Alternatively, the base-paired stem region may comprise complementary sequences corresponding to a selected promoter region, resulting in silencing of a coding sequence operably linked to said selected promoter. See, for example, Mette, et al., (2000) EMBO J. 19(19):5194-5201. Thus, the base-paired stem region of the molecule generally determines the specificity of the RNA interference. hpRNA molecules are highly efficient at inhibiting the expression of endogenous genes, and the RNA interference they induce is inherited by subsequent generations of plants. See, for example, Chuang and Meyerowitz, (2000) Proc. Natl. Acad. Sci. USA 97:4985-4990; Stoutjesdijk, et al., (2002) Plant Physiol. 129:1723-1731 and Waterhouse and Helliwell, (2003) Nat. Rev. Genet. 4:29-38. Methods for using hpRNA interference to inhibit or silence the expression of genes are described, for example, in Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini, et al., BMC Biotechnology 3:7 and US Patent Application Publication Number 2003/0175965; each of which is herein incorporated by reference. A transient assay for the efficiency of hpRNA constructs to silence gene expression in vivo has been described by Panstruga, et al., (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith, et al., (2000) Nature 407:319-320. In fact, Smith, et al., show 100% suppression of endogenous gene expression using ihpRNA-mediated interference. Methods for using ihpRNA interference to inhibit the expression of endogenous plant genes are described, for example, in Smith, et al., (2000) Nature 407:319-320; Wesley, et al., (2001) Plant J. 27:581-590; Wang and Waterhouse, (2001) Curr. Opin. Plant Biol. 5:146-150; Waterhouse and Helliwell, (2003) Nat. Rev. Genet. 4:29-38; Helliwell and Waterhouse, (2003) Methods 30:289-295 and US Patent Application Publication Number 2003/0180945, each of which is herein incorporated by reference.

The expression cassette for hpRNA interference may also be designed such that the sense sequence and the antisense sequence do not correspond to an endogenous RNA. In this instance, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 02/00904, herein incorporated by reference.

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for histidine kinase). Methods of using amplicons to inhibit the expression of endogenous plant genes are described, for example, in Angell and Baulcombe, (1997) EMBO J. 16:3675-3684, Angell and Baulcombe, (1999) Plant J. 20:357-362 and U.S. Pat. No. 6,646,805, each of which is herein incorporated by reference.

In some examples, the polynucleotide expressed by the expression cassette of the invention is catalytic RNA or has ribozyme activity specific for the messenger RNA of histidine kinase. Thus, the polynucleotide causes the degradation of the endogenous messenger RNA, resulting in reduced expression of the histidine kinase. This method is described, for example, in U.S. Pat. No. 4,987,071, herein incorporated by reference.

In some examples of the invention, inhibition of the expression of one or more histidine kinases may be obtained by RNA interference by expression of a gene encoding a micro RNA (miRNA). miRNAs are regulatory agents consisting of about 22 ribonucleotides. miRNA are highly efficient at inhibiting the expression of endogenous genes. See, for example, Javier, et al., (2003) Nature 425:257-263, herein incorporated by reference.

For miRNA interference, the expression cassette is designed to express an RNA molecule that is modeled on an endogenous miRNA gene. The miRNA gene encodes an RNA that forms a hairpin structure containing a 22-nucleotide sequence that is complementary to another endogenous gene (target sequence). For suppression of histidine kinase expression, the 22-nucleotide sequence is selected from a histidine kinase transcript sequence and contains 22 nucleotides of said histidine kinase sequence in sense orientation and 21 nucleotides of a corresponding antisense sequence that is complementary to the sense sequence. miRNA molecules are highly efficient at inhibiting the expression of endogenous genes, and the RNA interference they induce is inherited by subsequent generations of plants.

In one example, the polynucleotide encodes a zinc finger protein that binds to a gene encoding a maize histidine kinase, resulting in reduced expression of the gene. In particular examples, the zinc finger protein binds to a regulatory region of a histidine kinase gene. In other examples, the zinc finger protein binds to a messenger RNA encoding a histidine kinase and prevents its translation. Methods of selecting sites for targeting by zinc finger proteins have been described, for example, in U.S. Pat. No. 6,453,242, and methods for using zinc finger proteins to inhibit the expression of genes in plants are described, for example, in US Patent Application Publication Number 2003/0037355; each of which is herein incorporated by reference.

In some examples of the invention, the polynucleotide encodes an antibody that binds to at least one maize histidine kinase, and reduces histidine kinase activity of the histidine kinase protein. In another example, the binding of the antibody results in increased turnover of the antibody-histidine kinase complex by cellular quality control mechanisms. The expression of antibodies in plant cells and the inhibition of molecular pathways by expression and binding of antibodies to proteins in plant cells are well known in the art. See, for example, Conrad and Sonnewald, (2003) Nature Biotech. 21:35-36, incorporated herein by reference.

In some examples of the present invention, the activity of a histidine kinase is reduced or eliminated by disrupting the gene encoding the histidine kinase. The gene encoding the histidine kinase may be disrupted by any method known in the art. For example, in one example, the gene is disrupted by transposon tagging. In another example, the gene is disrupted by mutagenizing maize plants using random or targeted mutagenesis, and selecting for plants that have reduced histidine kinase activity, histidine kinase protein levels and/or histidine kinase mRNA levels.

In one example of the invention, transposon tagging is used to reduce or eliminate the histidine kinase activity of one or more histidine kinases. Transposon tagging comprises inserting a transposon within an endogenous histidine kinase gene to reduce or eliminate expression of the histidine kinase. By “histidine kinase gene”, it is intended to mean the gene that encodes a histidine kinase according to the invention.

In this example, the expression of one or more maize histidine kinases is reduced or eliminated by inserting a transposon within a regulatory region or coding region of the gene encoding the histidine kinase. A transposon that is within an exon, intron, 5′ or 3′ untranslated sequence, a promoter, or any other regulatory sequence of a histidine kinase gene may be used to reduce or eliminate the expression and/or activity of the encoded histidine kinase.

Methods for the transposon tagging of specific genes in plants are well known in the art. See, for example, Maes, et al., (1999) Trends Plant Sci. 4:90-96; Dharmapuri and Sonti, (1999) FEMS Microbiol. Lett. 179:53-59; Meissner, et al., (2000) Plant J. 22:265-274; Phogat, et al., (2000) J. Biosci. 25:57-63; Walbot, (2000) Curr. Opin. Plant Biol. 2:103-107; Gai, et al., (2000) Nucleic Acids Res. 28:94-96; Fitzmaurice, et al., (1999) Genetics 153:1919-1928). In addition, the TUSC process for selecting Mu insertions in selected genes has been described in Bensen, et al., (1995) Plant Cell 7:75-84; Mena, et al., (1996) Science 274:1537-1540 and U.S. Pat. No. 5,962,764; each of which is herein incorporated by reference.

Additional methods for decreasing or eliminating the expression of endogenous genes in plants are also known in the art and can be similarly applied to the instant invention. These methods include other forms of mutagenesis, such as ethyl methanesulfonate-induced mutagenesis, deletion mutagenesis, and fast neutron deletion mutagenesis used in a reverse genetics sense (with PCR) to identify plant lines in which the endogenous gene has been deleted. For examples of these methods see, Ohshima, et al., (1998) Virology 243:472-481; Okubara, et al., (1994) Genetics 137:867-874 and Quesada, et al., (2000) Genetics 154:421-436; each of which is herein incorporated by reference. In addition, a fast and automatable method for screening for chemically induced mutations, TILLING (Targeting Induced Local Lesions In Genomes), using denaturing HPLC or selective endonuclease digestion of selected PCR products is also applicable to the instant invention. See, McCallum, et al., (2000) Nat. Biotechnol. 18:455-457, herein incorporated by reference.

Mutations that impact gene expression or that interfere with the function (e.g., histidine kinase activity or phosphorelay activity) of the encoded protein are well known in the art. Insertional mutations in gene exons usually result in null-mutants. Mutations in conserved active site residues are particularly effective in inhibiting the histidine kinase activity of the encoded protein. Also, mutations in the histidine and aspartate residues that are involve in the phosphorelay function are also effective in inhibiting the activity of hybrid histidine kinases. Conserved active site residues of plant histidine kinases suitable for mutagenesis with the goal to eliminate histidine kinase activity and those required for the phosphorelay function have been described. See, for example, Hwang, et al., (2002) Plant Physiol. 129:500-515. Such mutants can be isolated according to well-known procedures, and mutations in different histidine kinase loci can be stacked by genetic crossing. See, for example, Gruis, et al., (2002) Plant Cell 14:2863-2882.

In another example of this invention, dominant mutants can be used to trigger RNA silencing due to gene inversion and recombination of a duplicated gene locus. See, for example, Kusaba, et al., (2003) Plant Cell 15:1455-1467.

The invention encompasses additional methods for reducing or eliminating the activity of one or more histidine kinases. Examples of other methods for altering or mutating a genomic nucleotide sequence in a plant are known in the art and include, but are not limited to, the use of RNA:DNA vectors, RNA:DNA mutational vectors, RNA:DNA repair vectors, mixed-duplex oligonucleotides, self-complementary RNA:DNA oligonucleotides and recombinogenic oligonucleobases. Such vectors and methods of use are known in the art. See, for example, U.S. Pat. Nos. 5,565,350; 5,731,181; 5,756,325; 5,760,012; 5,795,972 and 5,871,984; each of which are herein incorporated by reference. See also, WO 98/49350, WO 99/07865, WO 99/25821 and Beetham, et al., (1999) Proc. Natl. Acad. Sci. USA 96:8774-8778; each of which is herein incorporated by reference.

For the purposes of the present invention unless indicated otherwise or apparent from the context, a “subject plant” or “subject plant cell” is one in which genetic alteration, such as transformation, has been affected as to a gene of interest, or is a plant or plant cell which is descended from a plant or plant cell so altered and which comprises the alteration. A “control” or “control plant” or “control plant cell” provides a reference point for measuring changes in the subject plant or plant cell.

A control plant or control plant cell may comprise, for example: (a) a wild-type plant or plant cell, i.e., of the same genotype as the starting material for the genetic alteration which resulted in the subject plant or subject plant cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or subject plant cell; (d) a plant or plant cell genetically identical to the subject plant or subject plant cell but which is not exposed to conditions or stimuli that would induce expression of the gene of interest; or (e) the subject plant or subject plant cell itself, under conditions in which the gene of interest is not expressed.

For example, in various examples of the present invention, changes in histidine kinase activity, histidine kinase levels, cytokinin response, cytokinin perception and/or changes in one or more traits such as flowering time, seed set, branching, senescence, stress tolerance and root mass, could be measured by comparing a subject plant or subject plant cell to a control plant or control plant cell.

In certain examples the polynucleotides of the present invention can be stacked with any combination of polynucleotide sequences of interest in order to create plants with a desired phenotype. For example, the polynucleotides of the present invention may be stacked with any other polynucleotides encoding polypeptides having pesticidal and/or insecticidal activity, such as other Bacillus thuringiensis toxic proteins (described in U.S. Pat. Nos. 5,366,892; 5,747,450; 5,737,514; 5,723,756; 5,593,881 and Geiser, et al., (1986) Gene 48:109), lectins (Van Damme, et al., (1994) Plant Mol. Biol. 24:825, pentin (described in U.S. Pat. No. 5,981,722), and the like. The combinations generated can also include multiple copies of any one of the polynucleotides of interest. The polynucleotides of the present invention can also be stacked with any other gene or combination of genes to produce plants with a variety of desired trait combinations including, but not limited to, traits desirable for animal feed such as high oil genes (e.g., U.S. Pat. No. 6,232,529); balanced amino acids (e.g., hordothionins (U.S. Pat. Nos. 5,990,389; 5,885,801; 5,885,802; and 5,703,409); barley high lysine (Williamson, et al., (1987) Eur. J. Biochem. 165:99-106 and WO 98/20122) and high methionine proteins (Pedersen, et al., (1986) J. Biol. Chem. 261:6279; Kirihara, et al., (1988) Gene 71:359; and Musumura, et al., (1989) Plant Mol. Biol. 12:123)); increased digestibility (e.g., modified storage proteins (U.S. patent application Ser. No. 10/053,410, filed Nov. 7, 2001); and thioredoxins (U.S. patent application Ser. No. 10/005,429, filed Dec. 3, 2001)); the disclosures of which are herein incorporated by reference.

The polynucleotides of the present invention may be stacked with any gene or combination of genes and the combinations generated can include multiple copies of any one or more of the polynucleotides of interest. The desired combination may affect one or more traits; that is, certain combinations may be created for modulation of gene expression affecting cytokinin activity. Modulation of cytokinin sensing provided by the present application may be combined with methods and constructs to modulate cytokinin levels, such as those described in co-pending U.S. Patent Application Ser. Nos. 60/610,656, 60/637,230 and 60/696,405; 11/094,917; 10/817,483 and 09/545,334, incorporated herein by reference and in US Patent Application Publication Number 2003/0074698 (Schmulling, et al.) and U.S. Pat. No. 6,617,497 (Morris).

The polynucleotides of the present invention can also be stacked with traits desirable for disease or herbicide resistance (e.g., fumonisin detoxification genes (U.S. Pat. No. 5,792,931); avirulence and disease resistance genes (Jones, et al., (1994) Science 266:789; Martin, et al., (1993) Science 262:1432; Mindrinos, et al., (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)) and traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE), and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert, et al., (1988) J. Bacteriol. 170:5837-5847) facilitate expression of polyhydroxyalkanoates (PHAs)); the disclosures of which are herein incorporated by reference. One could also combine the polynucleotides of the present invention with polynucleotides providing agronomic traits such as male sterility (e.g., see U.S. Pat. No. 5,583,210), stalk strength, flowering time, or transformation technology traits such as cell cycle regulation or gene targeting (e.g., WO 99/61619, WO 00/17364, and WO 99/25821); the disclosures of which are herein incorporated by reference.

These stacked combinations can be created by any method including, but not limited to, cross-breeding plants by any conventional or TopCross methodology or genetic transformation. If the traits are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855 and WO99/25853, all of which are herein incorporated by reference.

Additionally, two protein coding regions may be operably linked in the same reading frame to produce a fusion protein. Some modifications may be made to facilitate the cloning, expression, or incorporation of a fusion protein. Such modifications are well known to those of skill in the art and include, for example, a methionine added at the amino terminus to provide an initiation site, or additional amino acids (e.g., poly His) placed on either terminus to create conveniently located purification sequences. Restriction sites or termination codons can also be introduced.

Expression of heterologous DNA sequences in a plant host is dependent upon the presence of an operably-linked promoter that is functional within the plant host. Choice of the promoter sequence will determine when and where within the plant the heterologous DNA sequence is expressed. Where continuous expression is desired throughout the cells of a plant, constitutive promoters are utilized. In contrast, where gene expression in response to a stimulus is desired, inducible promoters are the regulatory elements of choice. Where expression in specific tissues or organs is desired, tissue-preferred promoters are used. That is, these promoters can drive expression in specific tissues or organs. Additional regulatory sequences upstream and/or downstream from the core promoter sequence can be included in expression cassettes of transformation vectors to bring about varying levels of expression of heterologous nucleotide sequences in a transgenic plant. See, for example, U.S. Pat. No. 5,850,018.

Regulatory sequences may also be useful in controlling temporal and/or spatial expression of endogenous DNA.

Certain examples of the invention comprise nucleotide sequences that favor initiation of transcription in specific tissues, including vascular tissue and meristematic tissue of roots and/or shoots, and in callus tissue. One sequence exemplifying the promoter region of a histidine kinase of the present invention, AtCKI2, is set forth in SEQ ID NO.: 29.

By “promoter” is intended a regulatory region of DNA usually comprising a TATA box capable of directing RNA polymerase II to initiate RNA synthesis at the appropriate transcription initiation site for a particular coding sequence. A promoter can additionally comprise other recognition sequences generally positioned upstream or 5′ to the TATA box, referred to as upstream promoter elements, which influence the transcription initiation rate. It is recognized that having identified the nucleotide sequences for the promoter region disclosed herein, it is within the state of the art to isolate and identify further regulatory elements in the 5′ untranslated region upstream from the particular promoter region identified herein. Thus the promoter region disclosed herein is generally further defined by comprising upstream regulatory elements such as those responsible for tissue and temporal expression of the coding sequence, enhancers and the like. In the same manner, the promoter elements which enable expression in the desired tissue can be identified, isolated, and used with other core promoters to confirm tissue-preferred expression. Promoter elements may also be identified and isolated for use with other core promoters.

“Operably linked,” as used herein, includes reference to a functional linkage between a promoter and a second sequence, wherein the promoter sequence initiates and mediates transcription of the DNA corresponding to the second sequence. Generally, operably linked means that the nucleic acid sequences being linked are contiguous and, where necessary to join two protein coding regions, contiguous and in the same reading frame. An endogenous promoter is operably linked to the endogenous coding region which it regulates.

By “tissue-preferred” promoter is meant a sequence which preferentially initiates transcription in certain tissues, such as leaves, roots or seeds. A tissue-preferred promoter also may drive expression in certain tissues types in one or more organs; for example, in vascular tissues of roots or leaves.

The isolated promoter sequence of the present invention can be modified to provide for a range of expression levels of the heterologous nucleotide sequence. Less than the entire promoter region can be utilized and the ability to drive tissue-preferred expression retained. However, it is recognized that expression levels of mRNA can be decreased with deletions of portions of the promoter sequence. Thus, the promoter can be modified to be a weak or strong promoter. Generally, by “weak promoter” is intended a promoter that drives expression of a coding sequence at a low level. By “low level” is intended levels of about 1/10,000 transcripts to about 1/100,000 transcripts to about 1/500,000 transcripts. Conversely, a strong promoter drives expression of a coding sequence at a high level, or at about 1/10 transcripts to about 1/100 transcripts to about 1/1,000 transcripts. Generally, at least about 20 nucleotides of an isolated promoter sequence will be used to drive expression of a nucleotide sequence.

It is recognized that to increase transcription levels, enhancers can be utilized in combination with the promoter regions of the invention. Enhancers are nucleotide sequences that act to increase the expression of a promoter region. Enhancers are known in the art and include the SV40 enhancer region, the 35S enhancer element, and the like.

Fragments of a promoter nucleotide sequence disclosed herein are also encompassed by this invention. Such fragments will comprise at least about 20 contiguous nucleotides, preferably at least about 50 contiguous nucleotides, more preferably at least about 75 contiguous nucleotides, even more preferably at least about 100 contiguous nucleotides of the promoter nucleotide sequence disclosed herein. Such fragments will usually comprise the TATA recognition motif of the promoter sequence. Such fragments can be obtained by use of restriction enzymes to cleave the naturally-occurring promoter nucleotide sequences disclosed herein; by synthesizing a nucleotide sequence; through the use of PCR technology, and the like. See particularly, Mullis, et al., (1987) Methods Enzymol. 155:335-350 and Erlich, ed. (1989) PCR Technology (Stockton Press, New York).

Such fragments encompass, for example, sequences capable of driving tissue-preferred expression, elements responsible for temporal or tissue specificity, elements responsive to a phytohormone, and sequences useful as probes to identify similar sequences.

Biologically active variants of the promoter sequence are also encompassed by the composition of the present invention, including variants resulting from site-directed mutagenesis. A regulatory “variant” is a modified form of a regulatory sequence wherein one or more bases have been modified, removed or added. For example, a routine way to remove part of a DNA sequence is to use an exonuclease in combination with DNA amplification to produce unidirectional nested deletions of double stranded DNA clones. A commercial kit for this purpose is sold under the trade name Exo-Size™ (New England Biolabs, Beverly, Mass.). Briefly, this procedure entails incubating exonuclease III with DNA to progressively remove nucleotides in the 3′ to 5′ direction at 5′ overhangs, blunt ends or nicks in the DNA template. However, exonuclease III is unable to remove nucleotides at 3′, 4-base overhangs. Timed digestion of a clone with this enzyme produces unidirectional nested deletions.

One example of a regulatory sequence variant is a promoter formed by one or more deletions from a larger promoter. The 5′ portion of a promoter up to the TATA box near the transcription start site can be deleted without abolishing promoter activity, as described by Zhu, et al., The Plant Cell 7:1681-89 (1995). Such variants should retain promoter activity, particularly the ability to drive expression in specific tissues. Promoter activity can be measured by Northern blot analysis, reporter activity measurements when using transcriptional fusions, and the like. See, for example, Sambrook, et al., (1989) Molecular Cloning: A Laboratory Manual (2nd ed. Cold Spring Harbor Laboratory, Cold Spring Harbor, N.Y.), herein incorporated by reference.

A nucleotide sequence for the promoter of the invention, as well as fragments and variants thereof, can be provided in expression cassettes along with heterologous nucleotide sequences for expression in the plant of interest, more particularly in specific tissues of the plant. Such an expression cassette is provided with a plurality of restriction sites for insertion of the nucleotide sequence to be under the transcriptional regulation of the promoter. These expression cassettes are useful in the genetic manipulation of any plant to achieve a desired phenotypic response. This may be achieved by increasing expression of endogenous or exogenous products in the specific tissues of interest. Alternatively, there may be a reduction of expression of one or more endogenous products, particularly enzymes or cofactors.

The promoter region of the invention may be isolated from any plant, including, but not limited to, maize (Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum) and soybean (Glycine max). Promoter sequences from these or other plants may be isolated according to well-known techniques based on their sequence homology to the promoter sequence set forth herein. In these techniques, all or part of the known promoter sequence is used as a probe which selectively hybridizes to other sequences present in a population of cloned genomic DNA fragments (i.e. genomic libraries) from a chosen organism. Methods are readily available in the art for the hybridization of nucleic acid sequences.

The following examples are offered by way of illustration and not by way of limitation.

### Example 1

### Arabidopsis Histidine Kinases

Signal transduction systems mediate the perception of environmental and hormonal stimuli and the consequent downstream activation of appropriate cellular responses. The strategic location of these systems at the very beginning of signaling cascades makes them ideal candidates for the modulation of complex traits. The stimuli reported as being perceived by such systems in plants include osmolarity, ethylene and cytokinin, while those in bacteria include nitrogen, phosphate and salt, and those in cyanobacteria include salt and low temperature. A typical two-component system consists of a sensory histidine kinase and a response regulator.

The amino acid sequence of ZmCKI2 (SEQ ID NO: 8) is homologous to the Arabidopsis CKI2 protein (SEQ ID NO: 14) in the histidine kinase and response regulator domains, with overall sequence similarity of 55% across the entire protein (GAP; BLOSUM 62 matrix). The Arabidopsis CKI2 was originally isolated from an activation-tag screen for gain-of-function mutants that display a constitutive cytokinin response in the absence of the hormone (Kakimoto, (1996) Science 274:982-985). CKI2 may modulate cytokinin signal transduction by a unique mechanism relative to other described cytokinin receptors.

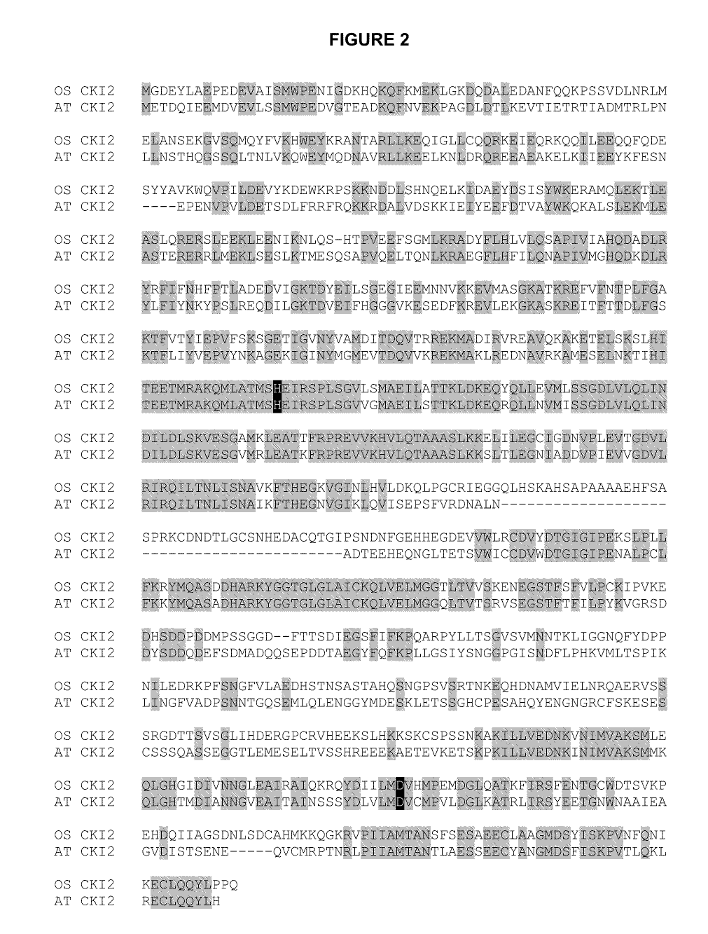

The AtCKI2 protein is a histidine kinase that functions as a component in signal transduction systems. The AtCKI2 coding region contains eleven introns, with one additional intron within the 5′ untranslated region. The longest open reading frame could be translated into a protein of 922 amino acid residues. The translation product of one predicted open reading frame from Oryza sativum (Os6G44410) was similar to CKI2 across its entire amino acid sequence and was designated as OsCki2 (see, FIG. 2 and SEQ ID NO: 23).

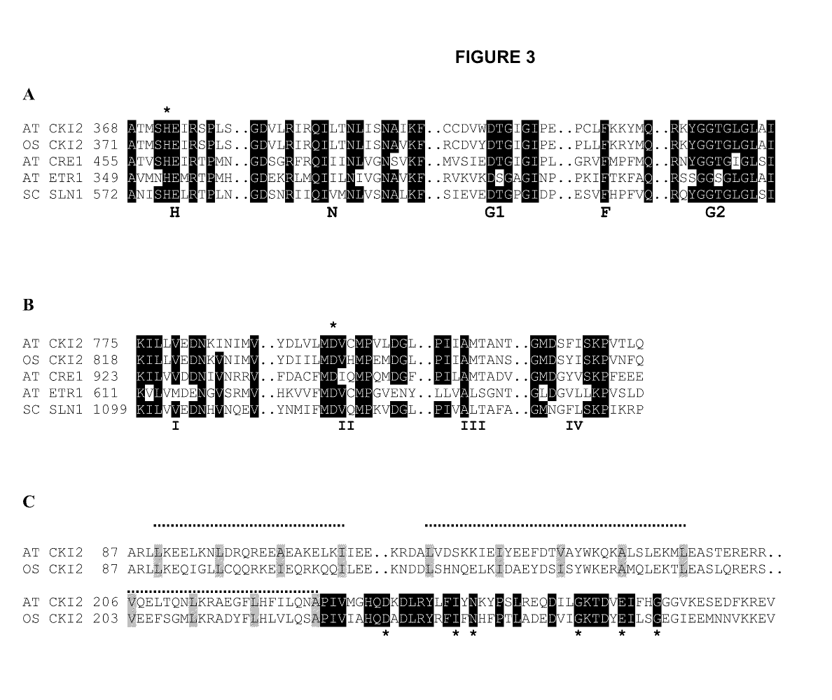

The AtCKI2 protein contains three regions with sequence identity to previously described protein domains. Demonstrably active histidine kinases have five motifs, the H, N, G1, F and G2 boxes, that are essential for ATP binding, hydrolysis and phosphorylation (Stock, et al., 2000). Hybrid-type receptor histidine kinases contain a second region, the response regulator region, which is similar to the signaling targets of the prototypical two-component signal transduction cascades. Response regulators have four motifs that contain residues essential for phosphorylation (Stock, et al., 2000). The predicted CKI2 translational products of both Arabidopsis and rice contain signature residues of the histidine kinase and response regulator regions that are apparent in hybrid type receptor histidine kinases (FIGS. 3A and 3B) from diverged organisms. Within plants, the hormone responsive cytokinin and ethylene receptors have been described as hybrid type receptor histidine kinases (Schaller, et al., 2002). The CKI2 protein, however, is distinct from these hormone receptors in that it lacks sequence similarity to the described cytokinin- or ethylene-binding protein domains within its amino terminus (Schaller and Bleecker, 1995; Yamada, et al., 2001). Further, data indicate that CKI2, similar to ETR1, can have multiple downstream targets.

A third region, near the CKI2 amino terminus, was identified with sequence similarity to the described PER/ANRT/SIM (PAS) domain superfamily, and contains signature residues that were identified in a comparison of PAS domains (FIG. 3C) (Taylor and Zhulin, 1999). A putative PAS core sequence is 59% identical (23 of 39 residues) between the Arabidopsis and rice sequences. Adjacent to the CKI2 PAS domain, three regions were identified with a repeating hydrophobic residue motif and a predicted alpha-helical tertiary structure.

The unique PAS-like domain of CKI2 is required for its ability to modulate cytokinin responsiveness. Based on an enhancer-trap line and expression profiling, CKI2 expression may be responsive to hypoxic growth conditions or hydrogen peroxide application (Desikan, et al., 2001; Baxter-Burrell, et al., 2003). Transcriptional autoregulation has been demonstrated for some bacterial and plant histidine kinases (Urao, et al., 1999; Bijlsma and Groisman, 2003; Rashotte, et al., 2003) and thus CKI2 could serve as an integrator of such environmental stimuli into phytohormone signal transduction. Consistent with this hypothesis, PAS domains of some proteins have been demonstrated to sense oxygen or redox potential (Taylor and Zhulin, 1999; Gilles-Gonzalez and Gonzalez, 2004). The presence of a CKI2-like PAS domain in some cyanobacterial histidine kinases enables the utilization of these model organisms to possibly identify and characterize the CKI2 stimulus.

A majority of receptor histidine kinases are membrane-localized with identifiable transmembrane regions within their amino termini (West and Stock, 2001; Hwang, et al., 2002). In contrast to other Arabidopsis histidine kinases, AtCKI2 appears to lack amino-terminal transmembrane regions based on structural prediction algorithms. To provide experimental evidence for its in vivo sub-cellular localization, constructs for the production of a near full-length or amino terminal region of CKI2 translationally fused to GFP were created. Similar to the GFP control, fluorescence of the CKI2(5-355):GFP fusion protein was localized to both the cytosol and nucleus during transient expression in onion epidermal cells. In contrast, fluorescence from a GFP fusion protein with the amino-terminal region of AHK1, AHK1(1-500):GFP, which contains identifiable transmembrane regions (Urao, et al., 1999), appeared to be localized to the plasma-membrane.

Several qualitative similarities between loss-of-function Arabidopsis mutant cki2-2 and the cre1ahk2ahk3 triple mutant (Nishimura, et al., (2004) Plant Cell 16:1365-1377) have been observed. Root growth is affected in both, with an overall reduction in root growth rate and alterations to root architecture. Overall vegetative growth was reduced; mutants had smaller leaves, reduced plant stature, and a delay in transition to reproductive development.

In Arabidopsis, AtCKI2 is preferentially expressed in a manner similar to specific expression domains of other cytokinin receptors; root, immature leaf and inflorescence tissue have the highest detectable expression levels. The endogenous CKI2 transcript could not be detected by northern hybridization using a total RNA blot. Employing RT-PCR, two adjacent regions of the 5′ CKI2 coding sequence, corresponding to the unique CKI2 amino terminus, could be amplified from cDNA derived from root-, leaf-, stem- or inflorescence-specific RNA. Utilizing 18S rRNA primers as an internal control, the cycle-dependent accumulation of the CKI2 product was reproducibly observed in fewer cycles in root and inflorescence samples, implying a higher level of endogenous expression in these tissues.

Transgenic Arabidopsis plants comprising a transcriptional fusion of AtCKI2 promoter (SEQ ID NO: 29), the coding region of the GUS reporter gene, and the PINII transcriptional termination region displayed histochemical staining for GUS activity predominantly in the vasculature of immature leaves, root vasculature, hypocotyl, and root meristem. In germinating seedlings, GUS activity was first detectable 48 hours after transferring to light in a diffuse pattern throughout the cotyledons and at the extreme root tip. The region of GUS activity in the root tip may include the root meristem but the root elongation zone proximal to the root tip was devoid of reporter gene activity. At 72 hours post-transfer, GUS activity appeared in the root vascular bundle, within regions that had presumably completed cellular differentiation (Scheres, et al., 2002). Additionally, GUS activity became more apparent in the vasculature of the cotyledons and was observed in the shoot meristem and adjacent hypocotyl. The spatial GUS activity of the root tip was recapitulated in lateral root development; GUS activity was first apparent throughout the primordium but later restricted to the distal tip and vascular bundle proximal to the main root. Although not detectable in emerging leaf primordia, PRO_{CKI2}:GUS transgene activity was seen diffusely through leaf tissue, including vasculature, in the early stages of development. Activity was detectable in the floral meristem and diffusely seen in all floral organs, being most pronounced in the vasculature. Within the gynoecium, GUS activity was absent during a brief period at anthesis and was not detectable in ovules, developing seeds or embryos.

The extreme root tip expression of CKI2 appears to be quite distinct from expression of other Arabidopsis histidine kinases. This is especially interesting as this region has been shown to be a site of both cytokinin and auxin accumulation based on immunolocalization and reporter gene analysis (Scheres, et al., 2002; Aloni, et al., 2004). Thus, CKI2 is not present during primary events of organogenesis, precluding its involvement in a developmental role, but is expressed in regions of hormone integration and may serve as a cellular constituent for competency to perceive such stimuli.

Of the currently examined response regulators, the unique root tip expression pattern of CKI2 may only be shared by ARR5 (D'Agostino, et al., 2000; Aloni, et al., 2004). In the absence of other two-component receptors in this region, CKI2 could serve as the primary initiator of two-component signal transduction, resulting in the transcriptional activation of ARR5. In other tissue types, such as shoot meristems and vasculature, which are reported to express several receptor histidine kinases, two-component signaling could be modulated by any of the described receptors.

This pattern of GUS expression in actively dividing cells is consistent with a function of CKI2 in cytokinin sensing. The vascular location of AtCKI2-promoter-driven GUS expression is similar to that of AtCRE1, AHK2 and AHK3, all of which are known to be involved in cytokinin sensing, as has been shown by Higuchi, et al., ((2004) Proc. Natl. Acad. Sci. USA 101:8821-8826) and Nishimura, et al., ((2004) Plant Cell 16:1365-1377).

Localization studies with ZmCKI2 in immature ear tissues of maize show distinct expression of ZmCKI2 in the vascular bundles, again implying a role for cytokinin sensing during transport of the hormone through vascular bundles.

PRO_{CKI2}:GUS activity was also assessed in response to exogenous hormone application in both seedlings and callus tissue. Five-day seedlings were incubated in the presence of either cytokinin or auxin and stained for GUS activity. Based on assays in various concentrations of GUS substrate, the spatial or quantitative activity of the PRO_{CKI2}:GUS transgene did not appear to be influenced by either a one or three hour period of hormone treatment. To examine chronic exposure to hormone application, hypocotyl segments of dark grown PRO_{CKI2}:GUS transgenics were cultured on callus inducing media (CIM). In dark-grown seedlings at five days growth, activity in the root tip and shoot meristem regions were similar to light-grown seedlings. However, GUS activity was relatively diffuse within the cotyledons, was only apparent in the region adjacent to the meristem within the hypocotyl, and was not detectable in the vasculature of the hypocotyl or root. Hypocotyls were excised and after seven days of growth on CIM, GUS activity was apparent within the vasculature of the entire hypocotyl, as well as small foci that appeared to correlate with regions of tissue proliferation. Hypocotyl segments were subsequently transferred to various ratios of cytokinin to auxin and assayed for activity at seven, fourteen and twenty-one days. GUS activity was similar under each of the hormone treatments for the entire time course and was reminiscent of observable activity in planta. Although not observed in developing shoot tissue, GUS activity was apparent in the root apex and vascular bundle, excluding the region immediately adjacent to the root meristem, of individually developing roots.

AtCKI2 can also interact with canonical two-component signaling intermediates as demonstrated by yeast two-hybrid assays. The heterologous yeast two hybrid assay has been successfully utilized to identify signaling targets of Arabidopsis histidine kinases (Urao, et al., 2000) and this assay was employed to define CKI2-dependent signaling cascades. The details of the assay are described below in Example 4.

CKI2 coding sequence fragments, containing either the amino terminus and PAS, or the histidine kinase and response regulator regions (both in combination and individually), were translationally fused to the GAL4 DNA binding domain (GAL4BD) coding sequence for use in a two hybrid screen. Of all examined fusion proteins (six total), only one (GAL4BD:CKI2(590-922) produced positive colonies from the primary screen that were successfully retested in both auxotrophic growth and colorimetric assays.

Clones representing two independent genes, ARABIDOPSIS HISTIDINE PHOSPHOTRANSFER PROTEIN 3 (AHP3, At5G39340) and At3G28690, a putative serine/threonine protein kinase, were identified from the library screen with GAL4BD:CKI2(590-922). AHPs are the described signaling targets of canonical receptor histidine kinases, such as CRE1, AHK1 and ETR1 (Grefen and Harter, 2004). Full-length proteins of four obtainable AHPs were subsequently assayed and showed positive histidine auxotrophic and colorimetric activity, indicating that AtCKI2 could interact with each of these proteins and further supporting the role of this histidine kinase in two-component signal transduction cascades. At3G28690 is homologous to the previously published ARABIDOPSIS PROTEIN KINASE 1 (APK1) (Hirayama and Oka, 1992) and APK2 (Ito, et al., 1997) and is designated APK3. The original screen identified a truncated APK3 protein fusion, APK3(262-476), which contains only part of the canonical kinase domain and carboxyl terminal region. Contrasting results with this truncation, fusion proteins with either the full-length APK3 or a second truncated protein, APK3(115-476), were unable to positively interact with GAL4BD:CKI2(590-922) in the two hybrid assay. This suggests that its amino-terminus may interfere with the CKI2 interaction. This observation could be due to the presence of an auto-inhibitory domain, as has been demonstrated for the unrelated protein kinase SOS2 (Zhu, 2002). APK3 has been classified as a Receptor Like Cytoplasmic Kinase (RLCK) VII (Family 1.2.2) in Arabidopsis. See, the Purdue University website on Functional Genomics of Plant Phosphorylation (plantsp.genomics.purdue.edu) Preliminary experiments suggest the endogenous APK3 is expressed at a relatively low level. Its use in modulation of cytokinin-dependent growth responses is being pursued through both transgenic and mutant analysis.

The first insertional allele of CKI2, cki2-1, was identified in a screen for cytokinin independent growth phenotypes of activation-tagged callus tissue. Cki2-1 callus could produce shoot tissue in the absence of exogenous cytokinin, but this phenotype was not observed in the progeny of regenerated plants (Kakimoto, 1996, 1998). The T-DNA of cki2-1 was inserted in the 5′ region of the coding sequence, likely producing a constitutively expressed truncated mRNA with the subsequent translational product lacking the 84 amino-terminal amino acid residues (CKI2(85-922)) (Kakimoto, 2002).

A T-DNA insertional line, herein designated as cki2-2, was characterized as a putative cki2 mutant allele. The left border of the T-DNA insertion could be amplified by PCR, and hemizygous plants were twice backcrossed to wild-type Arabidopsis (accession Columbia). An obvious mutant phenotype (see description below) cosegregated with plants that were homozygous for the T-DNA insertion. The segregation ratio of the mutant phenotype, 4.2:1 (wild type:mutant), was skewed relative to the expected ratio of 3:1 (Chi square test; p=0.05) for a single-gene recessive mutant allele, which could be due to a poorly penetrant negative effect on gametophyte development. The lack of observable GUS activity in female sporophytic or gametophytic tissue suggests that such defects may be limited to stamen or pollen development.

The left border genomic insertion site of the cki2-2 allele is located within the eleventh exon of CKI2 (FIG. 4A). This insertion site, confirmed using cki2-2 derived cDNA, would result in a translational product in which the 53 carboxyl-terminal residues of the endogenous CKI2 were replaced with 33 residues derived from the T-DNA left border. Two motifs critical to the formation of the phosphorylation active site of the response regulator domain (FIG. 3) would be lost in the cki2-2 translational product. Northern hybridizations and RT-PCR indicate that expression of the flanking coding sequence (At5G10730) appeared to be unaffected in cki2-2. Southern hybridization of cki2-2 genomic DNA with a cauliflower mosaic virus 35S promoter fragment, which is contained in the pROK2 T-DNA (Baulcombe, et al., 1986), suggested a tandem insertion occurred within the CKI2 coding sequence.

Thus, whereas cki2-1 was possibly unaffected in protein function, cki2-2 represents a functional null allele with regard to the response regulator activity. However, the cki2-2 protein may retain histidine kinase activity and the partially functional protein still may influence downstream signaling pathways. The ability of ETR1 histidine kinase region to function as an independent domain (Gamble, et al., 1998) and the observed phenotypic differences in histidine kinase or response regulator mutant versions of CKI1 in protoplast assays (Hwang and Sheen, 2001) supports such a proposition.

Further, Nakamura, et al., (1999) demonstrated that the CKI1 response regulator domain could facilitate trans-dephosphorylation of two AHP proteins, and response regulator domains of some bacterial histidine kinases are essential for defining protein interaction specificity or regulation of histidine kinase activity (Bijlsma and Groisman, 2003). It is feasible that the cki2 insertional mutants differentially influence cytokinin signaling through one of these mechanisms. Since these potential effects would occur post-translationally, detectable differences in reporter gene expression, such as cytokinin-inducible ARR6, may not be observable.

The cki2-2 mutant phenotype could be described as an overall reduction in the rate of plant growth and development. Cki2-2 seedlings could be identified based on a pale green color relative to wild type, which was apparent throughout its life cycle. Flowering time was lengthened in cki2-2 plants, with the transition to flowering occurring 12-15 days after wild type, although the number of vegetative leaves present at the transition (approximately twelve) was unaffected. Mature cki2-2 plants were reduced in both size and stature, averaging 25% primary shoot length of wild type, and had an apparent decrease in internode elongation. Based on seedling growth on plates, cki2-2 root growth was reduced relative to wild type. Lateral root initiation appeared to be temporally delayed in the mutant; however, unlike wild type, growth of adventitious primary roots could exceed that of the main root axis early in development. Gross morphological defects were not observed in any cki2-2 root, vegetative or floral organs and the skotomorphogenic response of dark grown cki2-2 seedlings appeared unaffected.

AtCKI2 has been previously associated with hormone signal transduction based on an activation-tagged line, and current observations suggest some overlapping expression domains with the described cytokinin receptors. To assess differences in hormone-dependent responses, cki2-2 seedlings were assayed for physiological and molecular responses to the cytokinin benzyladenine (BA) and the auxin indole acetic acid (IAA). Primary root growth is inhibited in wild type seedlings in response to increasing concentrations of both cytokinin and auxin (Inoue, et al., 2001); these phenotypic responses were similarly observed in cki2-2 seedlings. However, due to the relatively reduced cki2-2 root growth, this effect was less pronounced.

The hormone-dependent transcriptional activation of specific cytokinin- and auxin-inducible genes (D'Agostino, et al., 2000; Hagen and Guilfoyle, 2002), ARR6 and IAA5 respectively, was analyzed in both wild type and cki2-2. Seven-day-old seedlings were treated with BA or IAA in both wild type and cki2-2, and the hormone dependent induction of reporter gene expression was observable by semi-quantitative RT-PCR. The results indicate that cki2-2 seedlings respond to exogenous cytokinin and auxin application in both in planta physiological and molecular assays, suggesting that the loss of CKI2 activity does not fully abrogate the ability of the plant to sense these hormones.

Single mutants of the cytokinin hormone receptors CRE1 and AHK3 lack gross morphological defects when grown in normal growth conditions but are demonstrably cytokinin hyposensitive in callus growth assays (Inoue, et al., 2001; Ueguchi, et al., 2001; Higuchi, et al., 2004; Nishimura, et al., 2004). To further explore this observation, an AHK3-T-DNA insertion line was obtained, herein termed ahk3-4 (FIG. 4B), and transgenic lines constitutively expressing the full-length AHK3 coding sequence with the Zea mays UBIQUITIN promoter (PRO_{UBQ}) were created. The exonic location of the ahk3-4 T-DNA was confirmed, and constitutive expression of the transgenic lines was demonstrated by northern hybridization.

Duplicate, independent samples of hypocotyl tissue were excised from wild type, mutant, or transgenic Arabidopsis lines (wild type, ahk3-4, PRO_{UBQ}:AHK3, ahk1-1, PRO_{UBQ}:AHK1, cki2-2, PRO_{UBQ}:CKI2, PRO_{UBQ}:CKI2(1-363) and PRO_{UBQ}:CKI2(353-922)) and grown on plates containing differing cytokinin:auxin ratios. The following trans-zeatin concentrations were used in the gradient: 0.0, 0.01, 0.05, 0.1, 0.5, 1.0 ug/ml with 0.2 ug/ml indole butyric acid.

Plants of both the mutant and transgenic lines lacked apparent gross morphological defects under normal growth conditions. Under the tested callus growth conditions, significant differences in root formation between wild type, mutant and transgenic derived-callus were not apparent. In contrast, differences in shoot formation during callus tissue growth were observed relative to wild type callus. With a constant concentration of auxin, ahk3-4 callus required a higher concentration of cytokinin than wild type callus to produce significant tissue greening and shoot formation. In contrast, constitutive expression of AHK3 resulted in callus tissue that appeared green and initiated shoot formation at a lower cytokinin concentration relative to wild type. Thus, the relative level of functional AHK3 expression can influence some responses of callus tissue to exogenous cytokinin application but does not appear to alter plant development resulting from endogenous cytokinin levels.

The effects of altering the endogenous expression of the AHK1 histidine kinase, which lacks the described cytokinin-binding CHASE domain, in the hypocotyl growth assays were determined to serve as potential negative control. AHK1 has been suggested to function as an osmosensor in Arabidopsis (Urao, et al., 1999), possibly as a constitutively active histidine kinase and would hypothetically not influence the cytokinin-dependent growth of callus.

An ahk1 mutant, noted as ahk1-1 herein (FIG. 4C), was identified by screening a population of T-DNA insertional lines. PRO_{UBQ}:AHK1 transgenic lines were created and selected based on detectable transgene expression by northern hybridization. As seen with the AHK3 receptor histidine kinase, gross morphological defects of both mutant and transgenic lines were not observed under normal growth conditions. Similarly, in the callus growth assay, the hormone-dependent growth of both ahk1-1 mutant and PRO_{UBQ}:AHK1 transgenic calli appeared indistinguishable from wild type callus. These results suggest that alterations to AHK1 endogenous expression are unable to influence callus growth in either a positive or negative manner, demonstrating that not all Arabidopsis histidine kinases can modulate cytokinin responsiveness.

In similar experiments, the ability of CKI2 to influence callus growth was examined. Transgenic lines constitutively expressing the CKI2 genomic coding sequence, as confirmed by northern hybridization, were created. These lines lacked obvious morphological or growth defects under normal conditions.

Callus tissue of these cki2-2 mutant and PRO_{UBQ}:CKI2 transgenic lines were grown on plates containing an increasing ratio of cytokinin to auxin.

Overall, cki2-2 callus tissue appeared to be less prolific than wild type. Similar to ahk3-4 mutant callus, it appeared to be hyposensitive to the cytokinin concentration based on tissue greening and shoot formation. Constitutive CKI2 expression resulted in callus growth that appeared to be cytokinin hypersensitive, but relatively less pronounced than the PRO_{UBQ}:AHK3 transgenics.

To further explore the positive growth effects of CKI2 expression, transgenic tissue expressing either the amino terminal and PAS (PRO_{UBQ}:AT-CKI2(1-363)), or the histidine kinase and response regulator (PRO_{UBQ}:AT-CKI2(353-922)), coding sequences were assayed for differences in the callus growth. Expression of these two constructs did not appear to detectably influence hormone responsiveness of callus tissue.

Thus, analogous to the negative pleiotropic effects observed in normal growth conditions and similar to the hyposensitivity of ahk3-4 calli, cki2-2 calli are less responsive to the exogenous cytokinin concentration than wild type. The cytokinin hypersensitive effects of CKI2 expression, observed only in callus growth conditions, are phenotypically similar to constitutive AHK3 expression and reminiscent of the description of the CKI2 activation-tagged line (Kakimoto, 1996). This phenotype cannot be duplicated with expression of only the CKI2 histidine kinase and response regulator coding regions.

### Example 2

### Maize Histidine Kinases

The ZmCKI2 polynucleotide sequence (SEQ ID NO: 7) was obtained from a homology search of rice proteins using the Arabidopsis CKI2 protein sequence. The top rice candidate was used to search the maize genomic sequences that are available in public sequence databases. To produce the ZmCKI2 polynucleotide sequence, the identified partial 5′ and 3′-end maize sequences were assembled into a contig, and the missing middle regions were filled in by physical cloning using the end-sequence information. In particular, RNA was extracted from maize immature ear tissue, and a cDNA pool was prepared from the RNA using reverse transcription. The ZmCKI2 cDNA was cloned by straight PCR from this pool of cDNA.

The other polynucleotides of the invention that encode maize histidine kinases were obtained by a similar approach. ZmHK2, ZmHK3 and ZmCKI2 were physically cloned from the pool of cDNA prepared from immature ear RNA from maize as described above.

Sequence for ZmCRE1 (SEQ ID NO: 1-3) was completed through BAC screening and primer walking. Genomic sequence for a selected BAC clone was submitted to Sequence Annotation Viewer and was shown to contain a partial coding sequence for the 5′ end of ZmCRE1. This coding sequence showed perfect overlap with the full-insert sequence for a selected EST which encodes the 3′ end of ZmCRE1. The coding sequence identified from the BAC clone and the full-insert sequence from the EST were assembled to obtain full-length coding sequence for ZmCRE1.

Sequence for ZmCKI1 (SEQ ID NO: 26-28) was obtained based on partial sequence information gathered by genome walking. Based on partial sequence information for ZmCKI1 identified through BLAST searches, primers were designed which amplified a ˜3 kb fragment. Sequence confirmation was done on roughly 400 bp on either ends of this sequence, and when this sequence was used in BLAST searches, a 7008 bp genomic fragment was identified. This genomic sequence was submitted for cDNA prediction to Sequence Annotation Viewer and was predicted to contain the coding sequence for ZmCKI1.

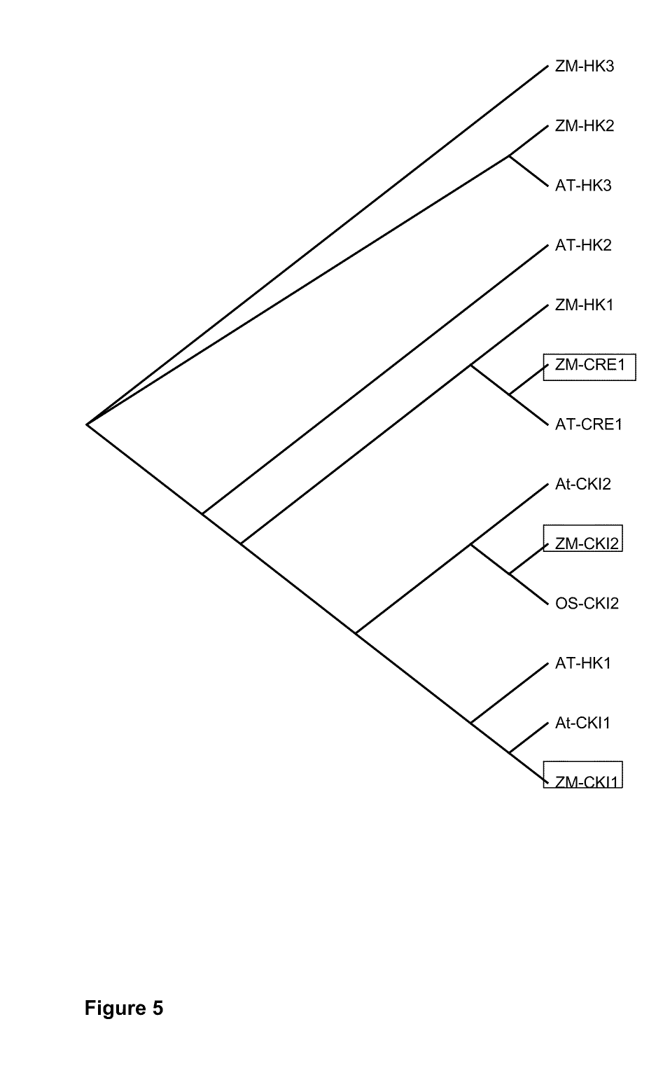

As observed with the Arabidopsis histidine kinase, AtCKI1, the ZmCKI1 coding region falls in the same clade as the osmosensing AHK1 (FIG. 5). This sequence similarity indicates that ZmCKI1 would be involved in cytokinin signaling as is proposed for AtCKI1, or in osmosensing as is proposed for AHK1. Homology searches also revealed that the ZmCKI1 sequence shows similarity to the cold-inducible histidine kinase from Catharanthus roseus.

A partial cDNA of ZmCKI1 was used to probe its cell-type specific expression in immature ears of B73. Similar to ZmCKI2, the expression of this gene within the immature ear was found to be confined to the vasculature.

The results of pairwise amino acid sequence comparisons of the ZmHK2, ZmHK3 and ZmCRE1 amino acid sequences with each of the AtCRE1, AtAHK2 and AtAHK3 amino acid sequences are provided in Table 1. The results of pairwise amino acid sequence comparisons of the ZmCKI2 amino acid sequence with each of the AtCKI2 and OsCKI2 amino acid sequences are provided in Table 2.

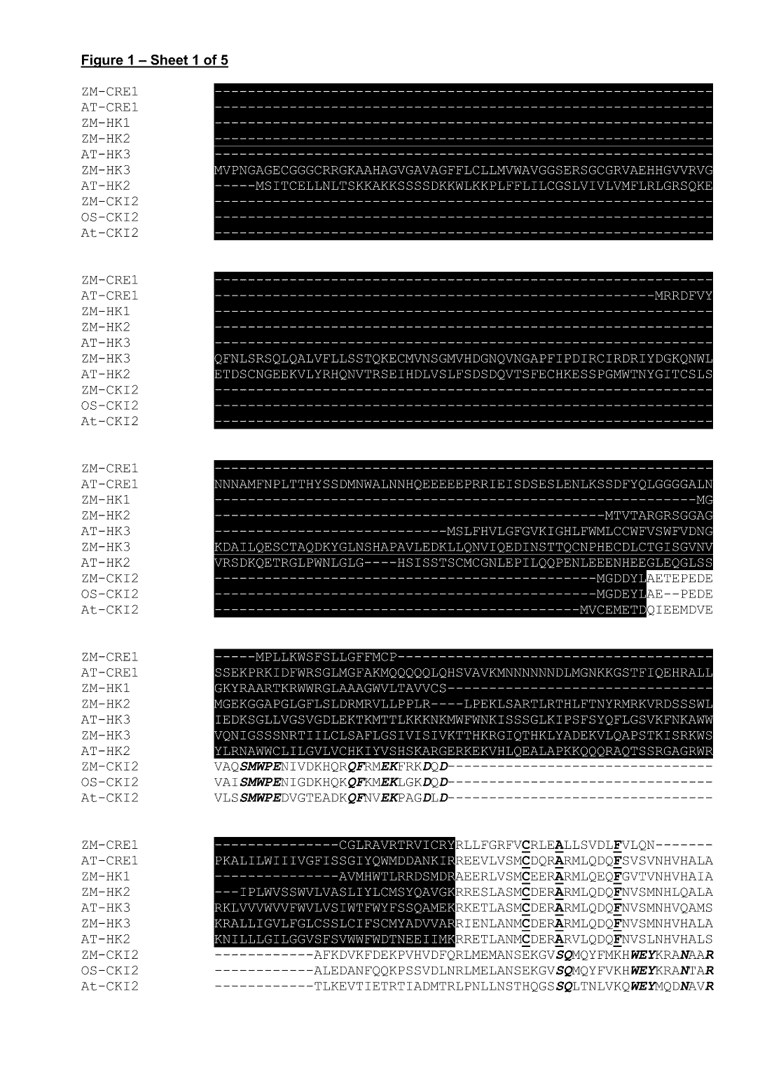

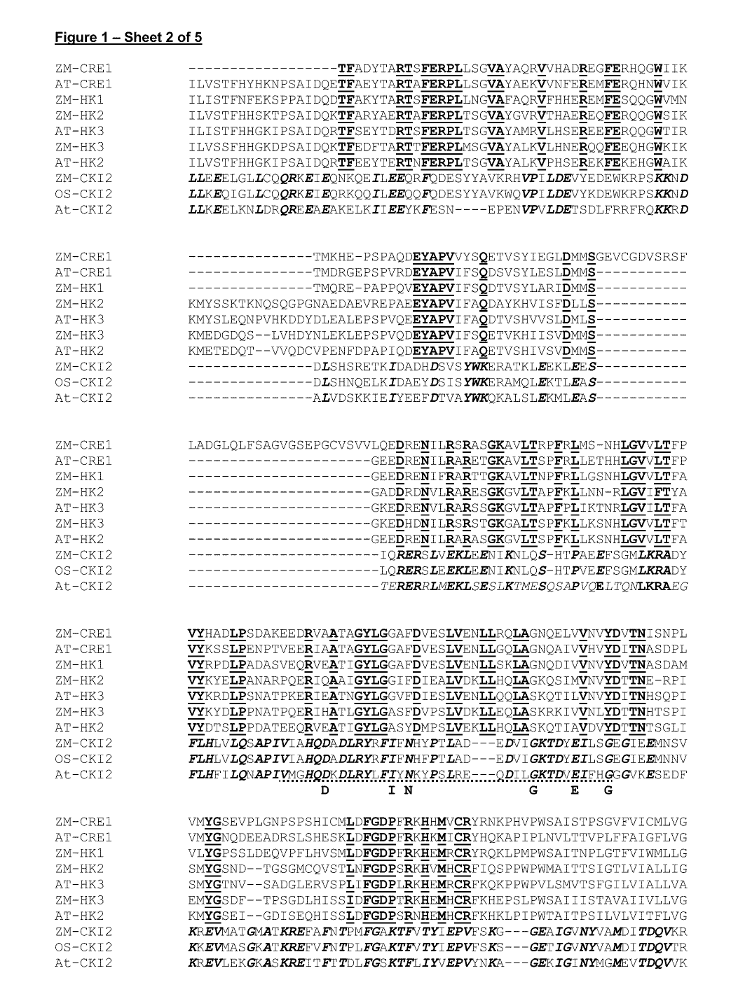

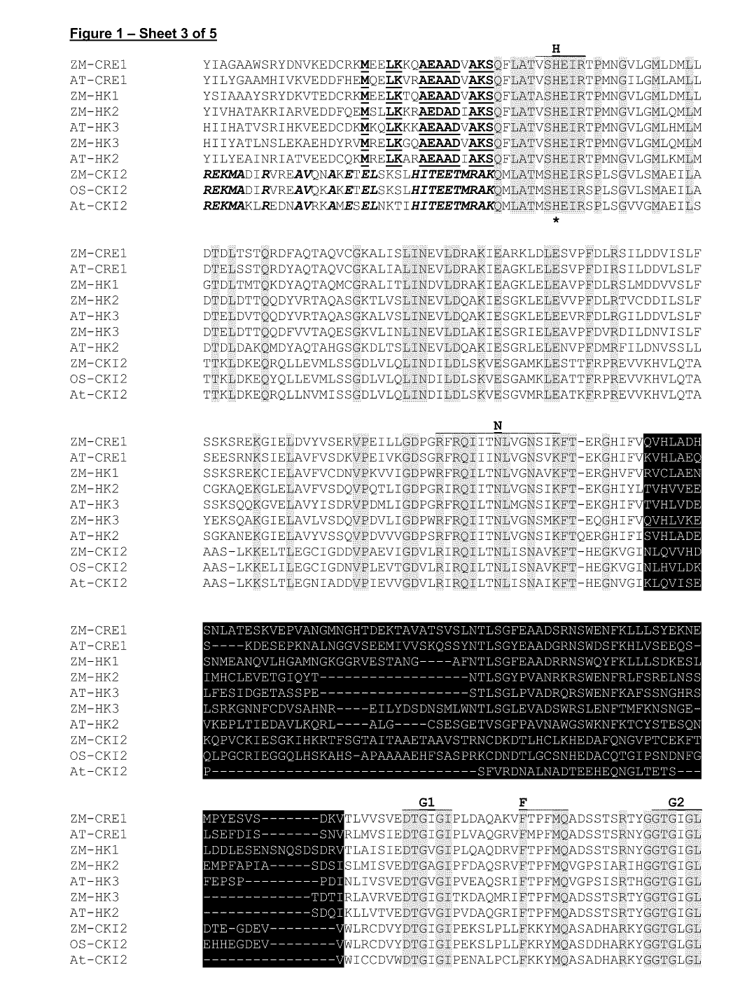

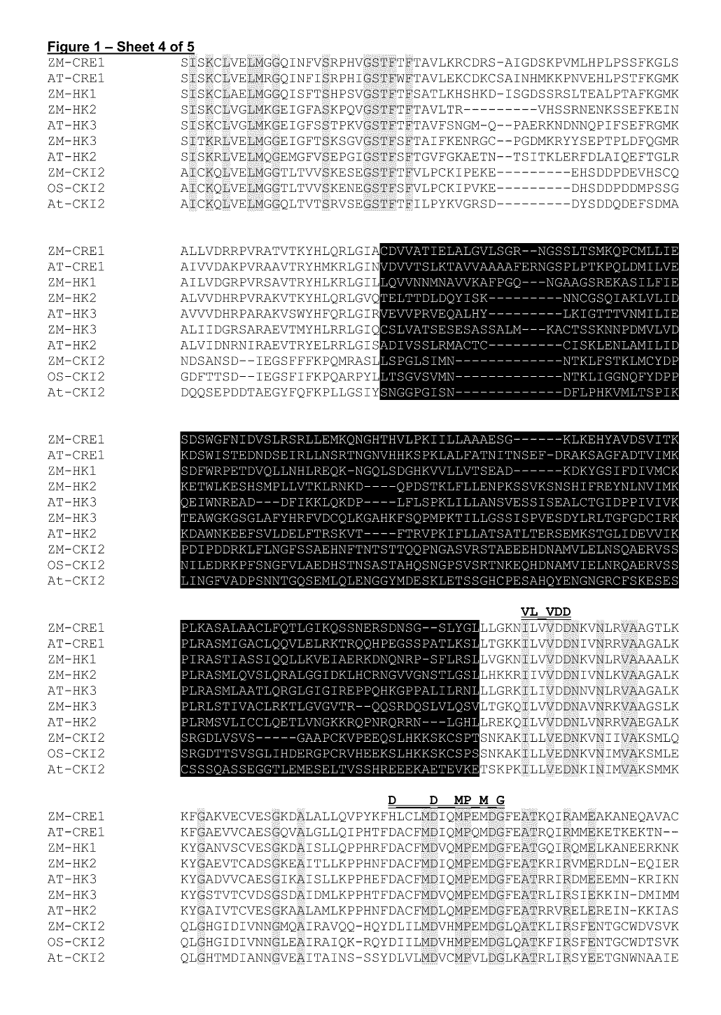

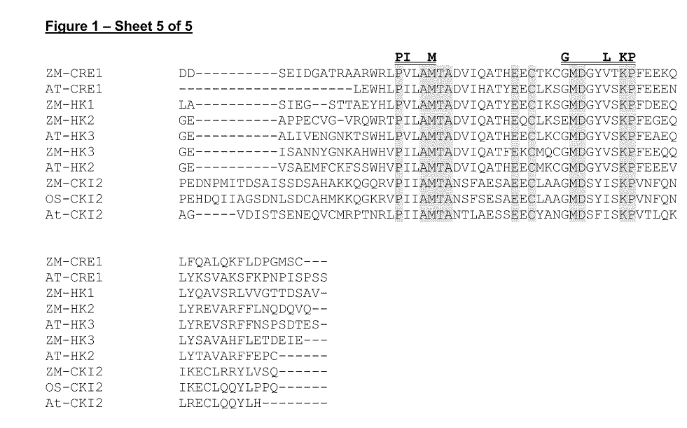

To determine the percent sequence identities presented in Tables 1 and 2, the amino acid sequences were aligned by GAP in pairwise combinations using the BLOSUM62 scoring matrix. In addition, a multiple amino acid sequence alignment of the ZmHK2, ZmHK3, ZmCKI2 and ZmCRE1 amino acid sequences with other hybrid-type receptor histidine kinases is provided in FIG. 1. The sequence identities in Tables 1 and 2 and the relatively high level of amino acid sequence conservation within the five histidine kinase boxes (H, N, G1, F, G2; see. FIGS. 1 and 3) provide further support for identification of sequences of the present invention as functional histidine kinases.

TABLE 1
Percent Amino Acid Sequence Identities
among Receptor Histidine Kinases

| | AtCRE1 | AtAHK2 | AtAHK3 |
|---|---|---|---|
| ZmHK2 | 52.6 | 54.2 | 60.1 |
| ZmHK3 | 50.6 | 53.2 | 56.8 |
| ZmCRE1 | 60.8 | 56.1 | 56.1 |

TABLE 2
Percent Amino Acid Sequence Identities
among Receptor Histidine Kinases

| | AtCKI2 | OsCKI2 |
|---|---|---|
| ZmCKI2 | 55.4 | 80.9 |

Furthermore, both the ZmHK2 and ZmHK3 proteins of the present invention have the conserved cytokinin-binding CHASE domain as shown in FIG. 1, further supporting their role in cytokinin sensing. Yonekura-Sakakibara, et al., ((2004) Plant Physiol. 134:1654-1661) demonstrated that similar ZmHK2 and ZmHK3 proteins are involved in cytokinin sensing. The nucleotide and amino acid sequences of the ZmHK2 (AB102956) and ZmHK3 (AB102957) proteins utilized by Yonekura-Sakakibara, et al., are similar, but not identical, to the respective ZmHK2 (SEQ ID NOS: 4-6) and ZmHK3 (SEQ ID NOS: 30-32) sequences of the present invention.

### Example 3

### Methods of Use of the Polynucleotides of the Invention

The polynucleotides of the invention can be used to alter the phenotype of plants. For example, a cytokinin-sensing histidine kinase of the present invention, when expressed under the direction of a tissue-preferred promoter in transgenic maize, will allow the increased sensing of the available cytokinin levels, leading to enhanced cytokinin responses in selected tissues. Combining a cytokinin sensor with a cytokinin biosynthetic gene (such as isopentenyl transferase) in tissue-preferred expression in transgenic maize will allow increased responses to the increased amount of cytokinin produced. Increased sensing of available cytokinin could also be combined with decreased expression of a cytokinin-degrading enzyme, such as cytokinin oxidase, in selected tissues. If the cytokinin sensor is downregulated, cytokinin responses can be reduced; this could be useful, for example, in roots, as cytokinins normally inhibit root growth.

Further, by introducing into a plant a polynucleotide of the invention comprising a functional histidine kinase coding sequence, a histidine kinase can be overexpressed in the plant, inducing the typical plant response to an environmental or hormonal stimulus in the absence of that stimulus. For example, overexpression of CKI1 or CKI2 in Arabidopsis induces typical cytokinin responses such as shoot formation from callus, cell proliferation, and the like, in the absence of cytokinin in the medium (Kakimoto, (1996) Science 274:982-985). Female reproductive tissue and/or the photosynthetic apparatus, for example, can be chosen for the overexpression of a histidine kinase. In the former, the enhanced cytokinin perception could lead to increased ear growth, and in the latter it could lead to reduced or delayed senescence.

Furthermore, the polynucleotides of the invention, comprising either full-length or partial-length histidine kinase coding sequences, can be used to down-regulate histidine kinase expression in a plant through the use of antisense and/or RNAi constructs. By down-regulating histidine kinases in this manner, a plant's normal response to an environmental or hormonal stimulus can be inhibited. For example, downregulating ZmCKI2 in roots through the use of a root-preferred promoter may alter normal cytokinin responses in roots and thus allow increased root growth.

Additionally, the polynucleotides of the invention can be used in methods for identifying other components of signal transduction cascades. In yeast two-hybrid assays using the specific protein domains encoded by the polynucleotides of the invention, proteins that interact in vivo with the histidine kinases of the invention can be identified. Such interacting proteins may be crucial for the modification of particular complex traits. Also, these domains can be used as starting points to build protein interaction maps in the corresponding signal transduction pathways. Such information will aid in the identification of a protein or gene in a pathway that should be targeted for the regulation of a trait of interest in a plant.

### Example 4

### Yeast Two-Hybrid Assays

The yeast two-hybrid assays discussed in Example 1 were conducted as follows. Polyadenylated mRNA was isolated from Arabidopsis aerial tissue using a FastTrack™ mRNA kit (Invitrogen, Carlsbad, Calif., USA). First strand cDNA was created using a cDNA synthesis kit (Stratagene, La Jolla, Calif., USA), subsequently cloned into pGADT7 (Clontech, Palo Alto, Calif., USA), and the cDNA library was transformed into the yeast strain AH109 (Clontech). AtCKI2 fragments were PCR amplified, inserting SfiI and BamHI sites at the 3′ and 5′ termini respectively, cloned into a pGBKT7 (Clontech) derivative, pRSASKIII, and sequenced. pRSASKIII derivatives, containing AT-CKI2(5-367), pRM242; AT-CKI2(357-922), pRM291; AT-CKI2(5-205), pRM362; AT-CKI2(200-367), pRM363; AT-CKI2(357-615), pRM431; AT-CKI2(590-922), pRM430, were transformed into Y187UH, a Y187 (Clontech) derivative with GAL4::HIS reporter gene inserted into the ura locus, and assayed for autoactivation of the HIS reporter. Y187UH, containing the pRSASKIII derivatives, and AH109, containing the cDNA library, were mated and allowed to grow on synthetic media lacking leucine, uracil, histidine and adenine. Plasmid DNA was isolated from viable transformants using an EZ Yeast plasmid kit (GenoTechnology, St. Louis, Mo., USA) and the pGADT7 insert was PCR amplified using vector specific primers that flanked the cloning site. PCR fragments were sequenced and vectors containing unique genes were transformed into Y187UH, with the respective pRSASKIII derivative, to confirm histidine auxotrophy and β-galactosidase activity. AT-AHP coding sequences were inserted into the SfiI/BamHI sites of pGADT7 as pRM751 (AHP1), pRM660 (AHP2), pRM661 (AHP3), pRM736 (AHP5).

### Example 5

### Transformation and Regeneration of Transgenic Maize Plants

Immature maize embryos from greenhouse donor plants are bombarded with a plasmid containing a maize histidine kinase polynucleotide of the invention operably linked to an ubiquitin promoter and the selectable marker gene PAT (Wohlleben, et al., (1988) Gene 70:25-37), which confers resistance to the herbicide Bialaphos. If desired, the maize promoters, zag2.1 (NCBI GenBank Accession Number X80206) or ckx1 (US Patent Application Publication Number 2002/0152500) can be used instead of the ubiquitin promoter. Alternatively, the selectable marker gene is provided on a separate plasmid. Transformation is performed as follows. Media recipes follow below.

### Preparation of Target Tissue

The ears are husked and surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5 cm target zone in preparation for bombardment.

### Preparation of DNA

A plasmid vector comprising a maize histidine kinase polynucleotide of the invention operably linked to an ubiquitin promoter is made. This plasmid DNA plus plasmid DNA containing a PAT selectable marker is precipitated onto 1.1 μm (average diameter) tungsten pellets using a CaCl₂ precipitation procedure as follows:

100 μl prepared tungsten particles in water

10 μl (1 μg) DNA in Tris EDTA buffer (1 μg total DNA)

100 μl 2.5 M CaCl₂

10 μl 0.1 M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

### Particle Gun Treatment

The sample plates are bombarded at level #4 in particle gun #HE34-1 or #HE34-2. All samples receive a single shot at 650 PSI, with a total of ten aliquots taken from each tube of prepared particles/DNA.

### Subsequent Treatment

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for increases or decreases in histidine kinase activity and/or histidine kinase protein levels.

### Bombardment and Culture Media

Bombardment medium (560Y) comprises 4.0 g/l N6 basal salts (SIGMA C-1416), 1.0 ml/l Eriksson's Vitamin Mix (1000×SIGMA-1511), 0.5 mg/l thiamine HCl, 120.0 g/l sucrose, 1.0 mg/l 2,4-D, and 2.88 g/l L-proline (brought to volume with D-I H₂O following adjustment to pH 5.8 with KOH); 2.0 g/l Gelrite (added after bringing to volume with D-I H₂O); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000×SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog, (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/1 myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

### Bombardment and Culture Media

Bombardment medium (560Y) comprises 4.0 g/l N6 basal salts (SIGMA C-1416), 1.0 ml/l Eriksson's Vitamin Mix (1000×SIGMA-1511), 0.5 mg/l thiamine HCl, 120.0 g/l sucrose, 1.0 mg/l 2,4-D, and 2.88 g/l L-proline (brought to volume with D-I H₂O following adjustment to pH 5.8 with KOH); 2.0 g/l Gelrite (added after bringing to volume with D-I H₂O) and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000×SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog, (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/1 myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

### Example 6

### The Production of Transformed Maize Plants via Agrobacterium-Mediated Transformation

For Agrobacterium-mediated transformation of maize with a maize histidine kinase polynucleotide of the invention, the method of Zhao is employed (U.S. Pat. No. 5,981,840 and PCT patent publication WO98/32326, the contents of which are hereby incorporated by reference). Briefly, immature embryos are isolated from maize and the embryos contacted with a suspension of Agrobacterium, where the bacteria are capable of transferring the maize histidine kinase polynucleotide of the invention to at least one cell of at least one of the immature embryos (step 1: the infection step). The embryos are co-cultured for a time with the Agrobacterium (step 2: the co-cultivation step). Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Next, inoculated embryos are cultured on medium containing a selective agent and growing transformed callus is recovered (step 4: the selection step). The callus is then regenerated into plants (step 5: the regeneration step).

### Example 7

### Soybean Embryo Transformation and Regeneration of Transformed Soybean Plants

Soybean embryos are bombarded with a plasmid containing a histidine kinase polynucleotide of the invention operably linked to constitutive promoter such as the Soybean Constitutive Promoter SCP1 (WO 97/47756, U.S. Pat. No. 6,555,673) for testing functionality or to a seed-specific promoter for transgenic modification of cytokinin sensing as follows. Alternatively, the maize promoters, zag2.1 or ckx1 can be used instead of the SCP1 promoter. To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein, et al., (1987) Nature (London) 327:70-73, U.S. Pat. No. 4,945,050). A Du Pont Biolistic PDS1000/HE instrument (helium retrofit) can be used for these transformations.

A selectable marker gene that can be used to facilitate soybean transformation is a transgene composed of the 35S promoter from Cauliflower Mosaic Virus (Odell, et al., (1985) Nature 313:810-812), the hygromycin phosphotransferase gene from plasmid pJR225 (from E. coli; Gritz, et al., (1983) Gene 25:179-188), and the 3′ region of the nopaline synthase gene from the T-DNA of the Ti plasmid of Agrobacterium tumefaciens. The expression cassette comprising the histidine kinase polynucleotide operably linked to the ubiquitin promoter can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1 M), and 50 μl CaCl₂ (2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 8

### Sunflower Meristem Tissue Transformation and Regeneration of Transgenic Sunflower Plants

Sunflower meristem tissues are transformed with an expression cassette containing a histidine kinase polynucleotide of the invention operably linked to an ubiquitin promoter as follows (see also, EP Patent Number EP 0 486233, herein incorporated by reference, and Malone-Schoneberg, et al., (1994) Plant Science 103:199-207). Mature sunflower seed (Helianthus annuus L.) are dehulled using a single wheat-head thresher. Seeds are surface sterilized for 30 minutes in a 20% Clorox bleach solution with the addition of two drops of Tween 20 per 50 ml of solution. The seeds are rinsed twice with sterile distilled water.

Split embryonic axis explants are prepared by a modification of procedures described by Schrammeijer et al. (Schrammeijer, et al., (1990) Plant Cell Rep. 9:55-60). Seeds are imbibed in distilled water for 60 minutes following the surface sterilization procedure. The cotyledons of each seed are then broken off, producing a clean fracture at the plane of the embryonic axis. Following excision of the root tip, the explants are bisected longitudinally between the primordial leaves. The two halves are placed, cut surface up, on GBA medium consisting of Murashige and Skoog mineral elements (Murashige, et al., (1962) Physiol. Plant. 15:473-497), Shepard's vitamin additions (Shepard, (1980) in Emergent Techniques for the Genetic Improvement of Crops (University of Minnesota Press, St. Paul, Minn.), 40 mg/l adenine sulfate, 30 g/l sucrose, 0.5 mg/l 6-benzyl-aminopurine (BAP), 0.25 mg/l indole-3-acetic acid (IAA), 0.1 mg/l gibberellic acid (GA3), pH 5.6, and 8 g/l Phytagar.

The explants are subjected to microprojectile bombardment prior to Agrobacterium treatment (Bidney, et al., (1992) Plant Mol. Biol. 18:301-313). Thirty to forty explants are placed in a circle at the center of a 60×20 mm plate for this treatment. Approximately 4.7 mg of 1.8 mm tungsten microprojectiles are resuspended in 25 ml of sterile TE buffer (10 mM Tris HCl, 1 mM EDTA, pH 8.0) and 1.5 ml aliquots are used per bombardment. Each plate is bombarded twice through a 150 mm nytex screen placed 2 cm above the samples in a PDS 1000® particle acceleration device.

Disarmed Agrobacterium tumefaciens strain EHA105 is used in all transformation experiments. A binary plasmid vector comprising the expression cassette that contains the histidine kinase polynucleotide operably linked to a constitutive promoter such as the Soybean Constitutive Promoter SCP1 for testing functionality or to a seed-specific promoter for transgenic modification of cytokinin sensing is prepared. Alternatively, the maize promoters, zag2.1 or ckx1 can be used instead of the SCP1 promoter. The binary plasmid vector is introduced into Agrobacterium strain EHA105 via freeze-thawing as described by Holsters, et al., (1978) Mol. Gen. Genet. 163:181-187. This plasmid further comprises a kanamycin selectable marker gene (i.e., nptII). Bacteria for plant transformation experiments are grown overnight (28° C. and 100 RPM continuous agitation) in liquid YEP medium (10 gm/l yeast extract, 10 gm/l Bactopeptone, and 5 gm/l NaCl, pH 7.0) with the appropriate antibiotics required for bacterial strain and binary plasmid maintenance. The suspension is used when it reaches an OD₆₀₀ of about 0.4 to 0.8. The Agrobacterium cells are pelleted and resuspended at a final OD₆₀₀ of 0.5 in an inoculation medium comprised of 12.5 mM MES pH 5.7, 1 gm/l NH₄Cl, and 0.3 gm/l MgSO₄.

Freshly bombarded explants are placed in an Agrobacterium suspension, mixed, and left undisturbed for 30 minutes. The explants are then transferred to GBA medium and co-cultivated, cut surface down, at 26° C. and 18-hour days. After three days of co-cultivation, the explants are transferred to 374B (GBA medium lacking growth regulators and a reduced sucrose level of 1%) supplemented with 250 mg/l cefotaxime and 50 mg/l kanamycin sulfate. The explants are cultured for two to five weeks on selection and then transferred to fresh 374B medium lacking kanamycin for one to two weeks of continued development. Explants with differentiating, antibiotic-resistant areas of growth that have not produced shoots suitable for excision are transferred to GBA medium containing 250 mg/l cefotaxime for a second 3-day phytohormone treatment. Leaf samples from green, kanamycin-resistant shoots are assayed for the presence of NPTII by ELISA and for the presence of transgene expression by assaying for histidine kinase activity as described elsewhere herein. NPTII-positive shoots are grafted to Pioneer® hybrid 6440 in vitro-grown sunflower seedling rootstock. Surface sterilized seeds are germinated in 48-0 medium (half-strength Murashige and Skoog salts, 0.5% sucrose, 0.3% gelrite, pH 5.6) and grown under conditions described for explant culture. The upper portion of the seedling is removed, a 1 cm vertical slice is made in the hypocotyl, and the transformed shoot inserted into the cut. The entire area is wrapped with parafilm to secure the shoot. Grafted plants can be transferred to soil following one week of in vitro culture. Grafts in soil are maintained under high humidity conditions followed by a slow acclimatization to the greenhouse environment. Transformed sectors of T₀ plants (parental generation) maturing in the greenhouse are identified by NPTII ELISA and/or by histidine kinase activity analysis of leaf extracts while transgenic seeds harvested from NPTII-positive T₀ plants are identified by histidine kinase activity analysis of small portions of dry seed cotyledon.

### Example 9

### Transient Expression of Histidine Kinase

The plasmid comprising a histidine kinase polynucleotide of the invention operably linked to a plant promoter is precipitated onto gold particles with polyethylimine (PEI; Sigma #P3143), while the transgenic expression cassette (UBI::moPAT˜GFPm::pinII) to be integrated is precipitated onto gold particles using the standard Ca⁺⁺ method. Briefly, coating gold particles with PEI is done as follows.

First, the gold particles are washed. Thirty-five mg of gold particles, for example 1.0 micron in average diameter (A.S.I. #162-0010), are weighed out in a microcentrifuge tube, and 1.2 ml absolute EtOH is added and vortexed for one minute. The tube is set aside for 15 minutes at room temperature and then centrifuged at high speed using a microfuge for 15 minutes at 4° C. The supernatant is discarded and a fresh 1.2 ml aliquot of EtOH is added, vortexed for one minute, centrifuged for one minute and the supernatant again discarded (this is repeated twice). A fresh 1.2 ml aliquot of EtOH is added, and this suspension (gold particles in EtOH) can be stored at −20° C. for weeks.

To coat particles with polyethylimine (PEI; Sigma #P3143), start with 250 μl of washed gold particle/EtOH, centrifuge and discard EtOH. Wash once in 100 μl ddH₂O to remove residual ethanol. Add 250 μl of 0.25 mM PEI, pulse-sonicate to suspend particles and then plunge tube into dry ice/EtOH bath to flash-freeze suspension into place. Lyophilize overnight. At this point, dry, coated particles can be stored at −80° C. for at least 3 weeks.

Before use, rinse particles 3 times with 250 μl aliquots of 2.5 mM HEPES buffer, ph 7.1, with 1× pulse-sonication and then quick vortex before each centrifugation. Suspend in final volume of 250 μl HEPES buffer. Aliquot 25 μl to fresh tubes before attaching DNA. To attach uncoated DNA, pulse-sonicate the particles, then add DNA's and mix by pipetting up and down a few times. Let sit for at least 2 minutes, spin briefly (e.g. 10 seconds), remove supernatant and add 60 μl EtOH. Spot onto macrocarriers and bombard following standard protocol. The Ca⁺⁺ precipitation and bombardment follows standard protocol for the PDS-1000.

The two particle preparations are mixed together; and the mixture is bombarded into plant cells (some cells receiving only a histidine kinase polynucleotide particle, some cells receiving only a PAT˜GFP particle and some cells receiving both). PEI-mediated precipitation results in a high frequency of transiently expressing cells and extremely low frequencies of recovery of stable transformants (relative to the Ca⁺⁺ method). Thus, the PEI-precipitated histidine kinase polynucleotide cassette expresses transiently and stimulates a burst of histidine kinase polynucleotide activity, but this plasmid does not integrate. The PAT˜GFP plasmid released from the Ca⁺⁺/gold particles integrates and expresses the selectable marker at a frequency that result in substantially improved recovery of transgenic events.

### Example 10

### Transient Expression of a Histidine Kinase Polynucleotide and Polypeptide

Transient expression of the histidine kinase polynucleotide product can be done by delivering histidine kinase 5′capped polyadenylated RNA, expression cassettes containing histidine kinase DNA, or histidine kinase protein. All of these molecules can be delivered using a biolistics particle gun. For example 5′capped polyadenylated histidine kinase RNA can easily be made in vitro using the mMessage mMachine® kit from Ambion (Austin, Tex., USA). Following the procedure outlined above, RNA is co-delivered along with DNA comprising a gene or gene fragment of agronomic interest and a marker used for selection/screening such as Ubi::moPAT˜GFPm::pinII. The cells receiving the RNA can be validated as being transgenic clonal colonies because they will also express the PAT˜GFP fusion protein (and thus will display green fluorescence under appropriate illumination). Plants regenerated from these embryos can then be screened for the presence of the gene of agronomic interest.

### Example 11

### Variants of Histidine Kinase

### A. Variant Nucleotide Sequences of SEQ ID NOS: 1, 3, 4, 6, 7, 9, 13, 15, 16, 18, 26, 28, and 32 That do not Alter the Encoded Amino Acid Sequence

The histidine kinase nucleotide sequences set forth in SEQ ID NOS: 1, 3, 4, 6, 7, 9, 13, 15, 16, 18, 26, 28, 30 and 32 are used to generate variant nucleotide sequences having the nucleotide sequence of the open reading frame with about 70%, 76%, 81%, 86%, 92% and 97% nucleotide sequence identity when compared to the starting unaltered ORF nucleotide sequence of SEQ ID NOS: 1, 3, 4, 6, 7, 9, 13, 15, 16, 18, 26, 28, 30 and 32, respectively. These functional variants are generated using a standard codon table. While the nucleotide sequence of the variant is altered, the amino acid sequence encoded by the open reading frame does not change.

### B. Variant Amino Acid Sequences of SEQ ID NOS: 2, 8, 14, 17, 23 and 27

Variant amino acid sequences of histidine kinases are generated. In this example, one amino acid is altered. Specifically, the open reading frame set forth in SEQ ID NOS: 2, 8, 14, 17, 23 or 27 is reviewed to determined the appropriate amino acid alteration. The selection of the amino acid to change is made by consulting the protein alignment (with the other orthologs and other gene family members from various species). See FIG. 1. An amino acid is selected that is deemed not to be under high selection pressure (not highly conserved) and which is rather easily substituted by an amino acid with similar chemical characteristics (i.e., similar functional side-chain). Using the protein alignment set forth in FIG. 1 an appropriate amino acid can be changed. Variants having about 70%, 75%, 81%, 86%, 92% and 97% nucleic acid sequence identity to SEQ ID NOS: 2, 8, 14, 17, 23 or 27 are generated using this method.

### C. Additional Variant Amino Acid Sequences of SEQ ID NOS: 2, 8, 14, 17, 23 and 27

In this example, artificial protein sequences are created having 82%, 87%, 92% and 97% identity relative to the reference protein sequence. This latter effort requires identifying conserved and variable regions from the alignment set forth in FIG. 1 and then the judicious application of an amino acid substitutions table. These parts will be discussed in more detail below.

Largely, the determination of which amino acid sequences are altered is made based on the conserved regions among plant histidine kinase proteins or among the other histidine kinase proteins. See FIG. 1. Based on the sequence alignment, the various regions of the histidine kinase that can likely be altered are represented in lower case letters, while the conserved regions are represented by capital letters. It is recognized that conservative substitutions can be made in the conserved regions below without altering function. In addition, one of skill will understand that functional variants of the histidine kinase sequence of the invention can have minor non-conserved amino acid alterations in the conserved domain.

The conserved regions of hybrid type receptor histidine kinases are evident in FIG. 1 and described in the brief description of FIG. 1 above.

Artificial protein sequences are then created that are different from the original in the intervals of 80-85%, 85-90%, 90-95% and 95-100% identity. Midpoints of these intervals are targeted, with liberal latitude of plus or minus 1%, for example. The amino acids substitutions will be effected by a custom Perl script. The substitution table is provided below in Table 3.

TABLE 3
Substitution Table

| | | Rank of | |
|---|---|---|---|
| Amino | Strongly Similar and | Order to | |
| Acid | Optimal Substitution | Change | Comment |
| I | L, V | 1 | 50:50 substitution |
| L | I, V | 2 | 50:50 substitution |
| V | I, L | 3 | 50:50 substitution |
| A | G | 4 | |
| G | A | 5 | |
| D | E | 6 | |
| E | D | 7 | |
| W | Y | 8 | |
| Y | W | 9 | |
| S | T | 10 | |
| T | S | 11 | |
| K | R | 12 | |
| R | K | 13 | |
| N | Q | 14 | |
| Q | N | 15 | |
| F | Y | 16 | |
| M | L | 17 | First methionine cannot |
| | | | change |
| H | | Na | No good substitutes |
| C | | Na | No good substitutes |
| P | | Na | No good substitutes |

First, any conserved amino acids in the protein that should not be changed is identified and “marked off” for insulation from the substitution. The start methionine will of course be added to this list automatically. Next, the changes are made.

H, C and P are not changed in any circumstance. The changes will occur with isoleucine first, sweeping N-terminal to C-terminal. Then leucine, and so on down the list until the desired target it reached. Interim number substitutions can be made so as not to cause reversal of changes. The list is ordered 1-17, so start with as many isoleucine changes as needed before leucine, and so on down to methionine. Clearly many amino acids will in this manner not need to be changed. L, I and V will involve a 50:50 substitution of the two alternate optimal substitutions.

The variant amino acid sequences are written as output. Perl script is used to calculate the percent identities. Using this procedure, variants of histidine kinases are generated having about 82%, 87%, 92% and 97% amino acid identity to the starting unaltered amino acid sequences of SEQ ID NOS: 2, 8, 14, 17, 23 or 27.

The article “a” and “an” as used herein refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

### CITATIONS

Aloni R, Langhans M, Aloni E, Ulirich C I (2004) Role of cytokinin in the regulation of root gravitropism. Planta 220: 177-182
Anantharaman V, Aravind L (2001) The CHASE domain: a predicted ligand-binding module in plant cytokinin receptors and other eukaryotic and bacterial receptors. Trends Biochem Sci 26: 579-582
Baulcombe D C, Saunders G R, Bevan M W, Mayo M A, Harrison M J (1986) Expression of biologically-active viral satellite RNA from the nuclear genome of transformed plants. Nature 321: 446-449
Baxter-Burrell A, Chang R, Springer P, Bailey-Serres J (2003) Gene and enhancer trap transposable elements reveal oxygen deprivation-regulated genes and their complex patterns of expression in Arabidopsis. Ann Bot (Lond) 91 Spec No: 129-141
Bijisma J J, Groisman E A (2003) Making informed decisions: regulatory interactions between two-component systems. Trends Microbiol 11: 359-366
D'Agostino I B, Deruere J, Kieber J J (2000) Characterization of the response of the Arabidopsis response regulator gene family to cytokinin. Plant Physiol 124: 1706-1717
Desikan R, A-H-Mackerness S, Hancock J T, Neill S J (2001) i Regulation of the Arabidopsis transcriptome by oxidative stress. Plant Physiol 127: 159-172
Gamble R L, Coonfield M L, Schaller G E (1998) Histidine kinase activity of the ETR1 ethylene receptor from Arabidopsis. Proc Natl Acad Sci USA 95: 7825-7829
Gilles-Gonzalez M A, Gonzalez G (2004) Signal transduction by heme-containing PAS-domain proteins. J Appl Physiol 96: 774-783
Grefen C, Harter K (2004) Plant two-component systems: principles, functions, complexity and cross talk. Planta 219: 733-742
Hagen G, Guilfoyle T (2002) Auxin-responsive gene expression: genes, promoters and regulatory factors. Plant Mol Biol 49: 373-385
Higuchi M, Pischke M S, Mahonen A P, Miyawaki K, Hashimoto Y, Seki M, Kobayashi M, Shinozaki K, Kato T, Tabata S, Helariutta Y, Sussman M R, Kakimoto T (2004) In planta functions of the Arabidopsis cytokinin receptor family. Proc Natl Acad Sci USA 101: 8821-8826
Hirayama T, Oka A (1992) Novel protein kinase of Arabidopsis thaliana (APK1) that phosphorylates tyrosine, serine and threonine. Plant Mol Biol 20: 653-662
Hwang I, Chen H C, Sheen J (2002) Two-component signal transduction pathways in Arabidopsis. Plant Physiol 129: 500-515
Hwang I, Sheen J (2001) Two-component circuitry in Arabidopsis cytokinin signal transduction. Nature 413: 383-389
Inoue T, Higuchi M, Hashimoto Y, Seki M, Kobayashi M, Kato T, Tabata S, Shinozaki K, Kakimoto T (2001) Identification of CRE1 as a cytokinin receptor from Arabidopsis. Nature 409: 1060-1063
Ito T, Takahashi N, Shimura Y, Okada K (1997) A serine/threonine protein kinase gene isolated by an in vivo binding procedure using the Arabidopsis floral homeotic gene product, AGAMOUS. Plant Cell Physiol 38: 248-258
Kakimoto T (1996) CKI1, a histidine kinase homolog implicated in cytokinin signal transduction. Science 274: 982-985
Kakimoto T (2002) Gene encoding protein participating in signal transduction of cytokinin. WO 2001/16332
Mougel C, Zhulin I B (2001) CHASE: an extracellular sensing domain common to transmembrane receptors from prokaryotes, lower eukaryotes and plants. Trends Biochem Sci 26: 582-584
Nakamura A, Kakimoto T, Imamura A, Suzuki T, Ueguchi C, Mizuno T (1999) Biochemical characterization of a putative cytokinin-responsive His-kinase, CKI1, from Arabidopsis thaliana. Biosci Biotechnol Biochem 63: 1627-1630
Nishimura C, Ohashi Y, Sato S, Kato T, Tabata S, Ueguchi C (2004) Histidine kinase homologs that act as cytokinin receptors possess overlapping functions in the regulation of shoot and root growth in Arabidopsis. Plant Cell 16: 1365-1377
Rashotte A M, Carson S D, To J P, Kieber J J (2003) Expression profiling of cytokinin action in Arabidopsis. Plant Physiol 132: 1998-2011
Schaller G E, Bleecker A B (1995) Ethylene-binding sites generated in yeast expressing the Arabidopsis ETR1 gene. Science 270: 1809-1811
Schaller G E, Mathews D E, Gribskov M, Walker J C (2002) Two-Component Signaling Elements and Histidyl-Aspartyl Phosphorelays. In C R Somerville, E M Meyerowitz, eds, The Arabidopsis Book. American Society of Plant Biologists
Scheres B, Benfey P, Dolan L (2002) Root Development. In C R Somerville, E M Meyerowitz, eds, The Arabidopsis Book. American Society of Plant Biologists, Rockville, Md.
Stock A M, Robinson V L, Goudreau P N (2000) Two-component signal transduction. Annu Rev Biochem 69: 183-215
Taylor B L, Zhulin I B (1999) PAS domains: internal sensors of oxygen, redox potential, and light. Microbiol. Mol Biol Rev 63: 479-506
Ueguchi C, Sato S, Kato T, Tabata S (2001) The AHK4 gene involved in the cytokinin-signaling pathway as a direct receptor molecule in Arabidopsis thaliana. Plant Cell Physiol 42: 751-755
Urao T, Miyata S, Yamaguchi-Shinozaki K, Shinozaki K (2000) Possible His to Asp phosphorelay signaling in an Arabidopsis two-component system. FEBS Lett 478: 227-232
Urao T, Yakubov B, Satoh R, Yamaguchi-Shinozaki K, Seki M, Hirayama T, Shinozaki K (1999) A transmembrane hybrid-type histidine kinase in Arabidopsis functions as an osmosensor. Plant Cell 11: 1743-1754
West A H, Stock A M (2001) Histidine kinases and response regulator proteins in two-component signaling systems. Trends Biochem Sci 26: 369-376
Yamada H, Suzuki T, Terada K, Takei K, Ishikawa K, Miwa K, Yamashino T, Mizuno T (2001) The Arabidopsis AHK4 histidine kinase is a cytokinin-binding receptor that transduces cytokinin signals across the membrane. Plant Cell Physiol 42: 1017-1023
Zhu J (2002) Salt and drought stress signal transduction in plants. Annu Rev Plant Biol 53: 247-273

That which is claimed:

1. An isolated polynucleotide comprising a nucleotide sequence selected from the group consisting of:
(a) a nucleotide sequence comprising SEQ ID NO: 1, 3, 4, 6, 7, 9, 13, 15, 16, 18, 26, 28, 30 or 32;
(b) a nucleotide sequence encoding an amino acid sequence comprising SEQ ID NO: 2, 5, 8, 14, 17, 27 or 31;
(c) a nucleotide sequence 90% identical to SEQ ID NO: 1, 3, 7, 9, 13, 15, 16, 18, 26 or 28;
(d) a nucleotide sequence encoding a polypeptide comprising at least 50 consecutive amino acids of SEQ ID NO: 2, 8, 14, 17 or 27, wherein said polypeptide retains histidine kinase activity; and
(e) a nucleotide sequence that hybridizes under stringent conditions to the complement of SEQ ID NO: 1, 3, 7, 9, 13, 15, 16, 18, 26 or 28, wherein said stringent conditions comprise hybridization in 50% formamide, 1 M NaCl, 1% SDS at 37° C., and at least one wash in 0.1×SSC at 60° C. to 65° C.

2. A method of modulating a plant response to cytokinin, comprising transforming said plant with a recombinant expression cassette comprising a polynucleotide of claim 1 operably linked to a promoter which drives expression in a plant.

3. The method of claim 2, wherein the promoter is a constitutive promoter.

4. The method of claim 2, wherein the promoter is a tissue-preferred promoter.

5. The method of claim 4, wherein the promoter directs expression in female reproductive tissue.

6. The method of claim 2, wherein the promoter is a cytokinin-inducible promoter.

7. The method of claim 2, wherein said modulation results in increased sensitivity to cytokinin.

8. The method of claim 2, wherein said plant is a monocot.

9. An isolated polypeptide comprising an amino acid sequence selected from the group consisting of:
(a) an amino acid sequence comprising SEQ ID NO: 2, 5, 8, 14, 17, 23, 27 or 31;
(b) an amino acid sequence comprising at least 70% sequence identity to SEQ ID NO: 2, 8, 14, 17, 23 or 27, wherein said polypeptide retains histidine kinase activity;
(c) an amino acid sequence encoded by a nucleotide sequence that hybridizes under stringent conditions to the complement of 1, 3, 7, 9, 13, 15, 16, 18, 26 or 28, wherein said stringent conditions comprise hybridization in 50% formamide, 1 M NaCl, 1% SDS at 37° C., and at least one wash in 0.1×SSC at 60° C. to 65° C.; and,
(d) an amino acid sequence comprising at least 50 consecutive amino acids of SEQ ID NO: 2, 8, 14, 17, 23 or 27, wherein said polypeptide retains kinase activity.

10. A method of modulating the histidine kinase activity in a plant comprising providing to said plant a polypeptide of claim 9.

11. A transformed plant comprising a polynucleotide operably linked to a promoter that drives expression in a plant, wherein said polynucleotide comprises a nucleotide sequence selected from the group consisting of:
(a) a nucleotide sequence comprising SEQ ID NO: 1, 3, 4, 6, 7, 9, 13, 15, 16, 18, 26, 28, 30 or 32;
(b) a nucleotide sequence encoding an amino acid sequence comprising SEQ ID NO: 2, 5, 8, 14, 17, 23, 27 or 31;
(c) a nucleotide sequence comprising at least 70% sequence identity to SEQ ID NO: 1, 3, 7, 9, 13, 15, 16, 18, 26 or 28, wherein said polynucleotide encodes a polypeptide with histidine kinase activity;
(d) a nucleotide sequence encoding an amino acid sequence comprising at least 70% sequence identity to SEQ ID NO: 2, 8, 14, 17 or 27, wherein said polynucleotide encodes a polypeptide comprising histidine kinase activity;
(e) a nucleotide sequence that hybridizes under stringent conditions to the complement of SEQ ID NO: 1, 3, 7, 9, 13, 15, 16, 18, 26 or 28, wherein said stringent conditions comprise hybridization in 50% formamide, 1 M NaCl, 1% SDS at 37° C., and at least one wash in 0.1×SSC at 60° C. to 65° C.; and
(f) a nucleotide sequence that is fully complementary to at least one sequence selected from the group consisting of the nucleotide sequences of (a)-(e).

12. The plant of claim 11, wherein said plant is a monocot.

13. The plant of claim 12, wherein said monocot is maize, wheat, rice, barley, sorghum or rye.

14. The plant of claim 11, wherein said plant is a dicot.

15. The plant of claim 14, wherein the dicot is soybean, Brassica, sunflower, cotton, Arabidopsis or alfalfa.

16. The plant of claim 11, wherein said polynucleotide is stably incorporated into the genome of the plant.

17. A transformed cell of the plant of claim 11.

18. A transformed seed of the plant of claim 11.

19. A method for modulating the level or activity of a polypeptide in a plant comprising transforming said plant with a construct comprising a fragment of SEQ ID NO: 1, 3, 4, 6, 7, 9, 13, 15, 16, 18, 26, 28, 30 or 32, or a fragment of the complement of any of the same, wherein expression of said fragment disrupts transcription or translation of the corresponding endogenous polynucleotide or an endogenous polynucleotide 90% identical thereto.

20. The method of claim 19, wherein said polypeptide is a histidine kinase.


SEQUENCE LISTING (37 sequences)

SEQ ID NO: 1 - DNA, 3590 bp, Zea mays
  CDS (1)...(2901): ZmCRE1
atgccacttcttaaatggagtttttctctacttggtttttttatgtgcccctgcggcctgcgcgcagtgcgcacgcgcgtaatttgccgatacagattactgttcggccgttttgtttgtcggctggaggctttactttcggtcgatttgtttgttctgcagaacacgttcgcggactacaccgccaggacgtcgttcgagcggccgctgctgagcggggtggcgtacgcgcagcgggtggtgcacgccgacagggagggcttcgagcgccaccaggggtggatcatcaagaccatgaagcacgagccgtccccggcgcaggacgagtacgcgcccgttgtatactcgcaggagaccgtctcctacatcgaggggctcgacatgatgtccggcgaggtctgcggcgacgtttcccgttcgtttcttgccgatgggctgcagttattctctgcaggtgttggttctgaacctggttgcgtttctgtggtgctgcaggaggaccgtgagaacattctgaggtcaagagcatcggggaaggcagttctcactcgaccattccggctcatgtcgaatcacctgggtgtcgtattgacttttcctgtctatcatgcggatcttccttctgatgccaaggaggaggatcgtgttgctgccaccgcaggataccttgggggagcatttgatgtagaatcattagtggaaaatttgttgaggcagctagctggcaatcaggaattggtggtaaatgtttatgatgtcacaaacatttcgaaccctcttgtcatgtatggatcagaagttcctcttggcaacccctcaccatcgcacatctgcatgctagattttggtgatccattcagaaagcatcatatggtttgcagatacagaaacaagcctcatgtcccatggtctgcaatttctaccccatctggtgtctttgtcatatgtatgcttgtggggtacatcgcgggtgccgcttggagtcgttatgataatgttaaggaagattgccggaaaatggaggagctgaaaaaacaggcggaagcagccgatgttgctaaatctcagttccttgcaactgtttctcatgagatcagaacgcccatgaatggagttctaggaatgcttgatatgctgttagacactgacctaacctcaactcagagggattttgcacaaaccgctcaagtctgtggaaaggctttaatatcgctaatcaatgaagtgcttgacagagcgaaaattgaagccagaaagttggatcttgaatctgtaccatttgatctgagatccatccttgatgatgtcatctcattattctcttcaaagtcaagagagaagggaattgagcttgatgtatatgtctctgaaagagttcccgaaatcttgttgggtgatcctggaaggtttcgacagataattacaaatttagtgggcaactcaattaagttcacagaacggggacatatttttgtacaagttcatctggcagatcattcaaatcttgcaacagaatccaaagttgagccggtggctaatgggatgaatggacatacagatgagaaaactgctgtagcaaccagtgtttctctcaacacactaagtggttttgaagctgctgatagccgaaatagttgggaaaacttcaagcttttgctttcttatgagaaaaatgagatgccctatgaaagtgtatctgataaagttactcttgtagtaagtgtggaagatacaggaataggtataccattggatgcacaagccaaggtgttcacacctttcatgcaggctgatagttcgacttccaggacatatggtggtactggaattggattgagcatcagcaaatgtcttgttgaactaatgggtggtcagataaactttgtcagccgaccacatgttgggagtacgttcacatttactgcggttctcaaaagatgtgacagaagcgctattggtgacagtaagcctgttatgttgcatcctcttccatccagtttcaaaggtttatctgcactattggttgatagaagaccagtaagagctactgtaactaagtatcacttgcaaaggctgggaattgcctgtgatgttgttgctaccattgaactggctcttggtgtgctgtccgggagaaatggcagttccctaaccagtatgaagcaaccatgcatgttattgatcgagagtgattcatggggcttcaatattgatgtttctttacgatctagactcctggagatgaagcagaatggtcacacacatgtattacccaaaatcatccttcttgcagctgcagaatcgggcaaactcaaagaacactatgcagttgattctgtgatcacgaaacctctgaaagcaagcgcacttgccgcttgtctattccaaacacttggtatcaaacagtcaagcaacgagagaagtgacaactcaggttctctttacgggttgcttcttggcaagaacatattggtggttgatgacaacaaggtaaatcttagagtggctgctggcacgttaaagaaatttggagcaaaggtggagtgcgtggagagtggaaaagatgccctcgccctcctacaggtcccatataagtttcatctttgtctcatggacattcagatgcccgaaatggatgggtttgaggctaccaagcaaataagggcaatggaagcgaaggcaaatgagcaggcagtcgcctgtgacgattcagaaatagatggcgcgacaagggcagcaagatggcgcctgcctgttcttgcaatgaccgccgatgtcatccaggccacccatgaagagtgcacgaagtgcgggatggatgggtacgtcacaaagcccttcgaggagaagcagctcttccaggcgttgcagaagttcttggaccccggcatgtcctgctaacgccgaagtgatgcgttggaggtgaggcactattctcctctattctgtaattccctaaccttggccgcaacctgatccccaagaaagaccgtgcagcgcgggctgctcgtcgatcatgccagcggggaacagcaggcagacaaggcagcacgcaccacgggccttcgctggtctgcctgcctgtgcaggcgtcgtgtaaatgtattgtatttgcagggatactgtttacagcgctagtatgccccgtgccacaatgtccgagctacctgtggctgatgcacgttgcgcataggtggtttttttttgtacagcttccaccatttttgtgctccagatcagaccaccgaaggaggcaagcatgctcgctggtgcagtgcagcactggaaggaaaacctgacttcctgagagtagatttttgctgtttaattgcattgggatttatgatgtattataatcagataaatgtgtgatgtaagctggcgtaattggcccaatgcatttccattgttatggatgagttgaaatggcattggttagactacactattgggtgtatgcccttccacagttccattttccctgcaaaatatcaaaatgtgcaaagggatagtggaatcatggcttgaattatatgaattttctttggtgttttaaaaaaaaaaaaaaaaaaaaaaaaaaaaaaaaaaaa

SEQ ID NO: 2 - protein, 966 aa, Zea mays
MPLLKWSFSLLGFFMCPCGLRAVRTRVICRYRLLFGRFVCRLEALLSVDLFVLQNTFADYTARTSFERPLLSGVAYAQRVVHADREGFERHQGWIIKTMKHEPSPAQDEYAPVVYSQETVSYIEGLDMMSGEVCGDVSRSFLADGLQLFSAGVGSEPGCVSVVLQEDRENILRSRASGKAVLTRPFRLMSNHLGVVLTFPVYHADLPSDAKEEDRVAATAGYLGGAFDVESLVENLLRQLAGNQELVVNVYDVTNISNPLVMYGSEVPLGNPSPSHICMLDFGDPFRKHHMVCRYRNKPHVPWSAISTPSGVFVICMLVGYIAGAAWSRYDNVKEDCRKMEELKKQAEAADVAKSQFLATVSHEIRTPMNGVLGMLDMLLDTDLTSTQRDFAQTAQVCGKALISLINEVLDRAKIEARKLDLESVPFDLRSILDDVISLFSSKSREKGIELDVYVSERVPEILLGDPGRFRQIITNLVGNSIKFTERGHIFVQVHLADHSNLATESKVEPVANGMNGHTDEKTAVATSVSLNTLSGFEAADSRNSWENFKLLLSYEKNEMPYESVSDKVTLVVSVEDTGIGIPLDAQAKVFTPFMQADSSTSRTYGGTGIGLSISKCLVELMGGQINFVSRPHVGSTFTFTAVLKRCDRSAIGDSKPVMLHPLPSSFKGLSALLVDRRPVRATVTKYHLQRLGIACDVVATIELALGVLSGRNGSSLTSMKQPCMLLIESDSWGFNIDVSLRSRLLEMKQNGHTHVLPKIILLAAAESGKLKEHYAVDSVITKPLKASALAACLFQTLGIKQSSNERSDNSGSLYGLLLGKNILVVDDNKVNLRVAAGTLKKFGAKVECVESGKDALALLQVPYKFHLCLMDIQMPEMDGFEATKQIRAMEAKANEQAVACDDSEIDGATRAARWRLPVLAMTADVIQATHEECTKCGMDGYVTKPFEEKQLFQALQKFLDPGMSC

SEQ ID NO: 3 - DNA, 2901 bp, Zea mays
  CDS (1)...(2901): ZmCRE1
atgccacttcttaaatggagtttttctctacttggtttttttatgtgcccctgcggcctgcgcgcagtgcgcacgcgcgtaatttgccgatacagattactgttcggccgttttgtttgtcggctggaggctttactttcggtcgatttgtttgttctgcagaacacgttcgcggactacaccgccaggacgtcgttcgagcggccgctgctgagcggggtggcgtacgcgcagcgggtggtgcacgccgacagggagggcttcgagcgccaccaggggtggatcatcaagaccatgaagcacgagccgtccccggcgcaggacgagtacgcgcccgttgtatactcgcaggagaccgtctcctacatcgaggggctcgacatgatgtccggcgaggtctgcggcgacgtttcccgttcgtttcttgccgatgggctgcagttattctctgcaggtgttggttctgaacctggttgcgtttctgtggtgctgcaggaggaccgtgagaacattctgaggtcaagagcatcggggaaggcagttctcactcgaccattccggctcatgtcgaatcacctgggtgtcgtattgacttttcctgtctatcatgcggatcttccttctgatgccaaggaggaggatcgtgttgctgccaccgcaggataccttgggggagcatttgatgtagaatcattagtggaaaatttgttgaggcagctagctggcaatcaggaattggtggtaaatgtttatgatgtcacaaacatttcgaaccctcttgtcatgtatggatcagaagttcctcttggcaacccctcaccatcgcacatctgcatgctagattttggtgatccattcagaaagcatcatatggtttgcagatacagaaacaagcctcatgtcccatggtctgcaatttctaccccatctggtgtctttgtcatatgtatgcttgtggggtacatcgcgggtgccgcttggagtcgttatgataatgttaaggaagattgccggaaaatggaggagctgaaaaaacaggcggaagcagccgatgttgctaaatctcagttccttgcaactgtttctcatgagatcagaacgcccatgaatggagttctaggaatgcttgatatgctgttagacactgacctaacctcaactcagagggattttgcacaaaccgctcaagtctgtggaaaggctttaatatcgctaatcaatgaagtgcttgacagagcgaaaattgaagccagaaagttggatcttgaatctgtaccatttgatctgagatccatccttgatgatgtcatctcattattctcttcaaagtcaagagagaagggaattgagcttgatgtatatgtctctgaaagagttcccgaaatcttgttgggtgatcctggaaggtttcgacagataattacaaatttagtgggcaactcaattaagttcacagaacggggacatatttttgtacaagttcatctggcagatcattcaaatcttgcaacagaatccaaagttgagccggtggctaatgggatgaatggacatacagatgagaaaactgctgtagcaaccagtgtttctctcaacacactaagtggttttgaagctgctgatagccgaaatagttgggaaaacttcaagcttttgctttcttatgagaaaaatgagatgccctatgaaagtgtatctgataaagttactcttgtagtaagtgtggaagatacaggaataggtataccattggatgcacaagccaaggtgttcacacctttcatgcaggctgatagttcgacttccaggacatatggtggtactggaattggattgagcatcagcaaatgtcttgttgaactaatgggtggtcagataaactttgtcagccgaccacatgttgggagtacgttcacatttactgcggttctcaaaagatgtgacagaagcgctattggtgacagtaagcctgttatgttgcatcctcttccatccagtttcaaaggtttatctgcactattggttgatagaagaccagtaagagctactgtaactaagtatcacttgcaaaggctgggaattgcctgtgatgttgttgctaccattgaactggctcttggtgtgctgtccgggagaaatggcagttccctaaccagtatgaagcaaccatgcatgttattgatcgagagtgattcatggggcttcaatattgatgtttctttacgatctagactcctggagatgaagcagaatggtcacacacatgtattacccaaaatcatccttcttgcagctgcagaatcgggcaaactcaaagaacactatgcagttgattctgtgatcacgaaacctctgaaagcaagcgcacttgccgcttgtctattccaaacacttggtatcaaacagtcaagcaacgagagaagtgacaactcaggttctctttacgggttgcttcttggcaagaacatattggtggttgatgacaacaaggtaaatcttagagtggctgctggcacgttaaagaaatttggagcaaaggtggagtgcgtggagagtggaaaagatgccctcgccctcctacaggtcccatataagtttcatctttgtctcatggacattcagatgcccgaaatggatgggtttgaggctaccaagcaaataagggcaatggaagcgaaggcaaatgagcaggcagtcgcctgtgacgattcagaaatagatggcgcgacaagggcagcaagatggcgcctgcctgttcttgcaatgaccgccgatgtcatccaggccacccatgaagagtgcacgaagtgcgggatggatgggtacgtcacaaagcccttcgaggagaagcagctcttccaggcgttgcagaagttcttggaccccggcatgtcctgctaa

SEQ ID NO: 4 - DNA, 3024 bp, Zea mays
  CDS (1)...(3021): ZmHK2
atgaccgttacggcgcgcgggaggagcggtggagccgggatgggggagaagggcggcgctcccgggctcggcttcctgagcctcgatcggatgcgcgtcctcctgccgccgctccgcctgccggagaagctgtcggccaggacgctgcggacgcacctcttcactaactatcggatgcgcaaggtgcgggacagctccagctggctcatcccgctctgggtcagctcttgggtcctcgtcgcctccctcatctacctctgcatgagctaccaggccgtcggcaagcgccgggagtctctcgccagcatgtgcgacgagcgcgcgcgcatgctccaggaccaatttaacgtcagcatgaaccacctccaggcgctcgccatcctcgtctccaccttccaccactccaagacaccctccgccatcgaccagaaaacgttcgcgaggtatgcggagaggacggcgttcgagcggccgctgacgagcggggtcgcgtacggggtgcgggtcacgcacaccgagcgggagcagttcgagcgccagcagggatggagcatcaagaagatgtactcctccaagaccaagaatcagtcgcagggacccgggaacgccgaggacgccgaggtgcgggagccggcggaggagtatgctcccgtaatcttcgcgcaggacgcctacaagcacgtcatctccttcgacttgctatccggcgctgatgaccgggacaacgtactccgagctagagaatctgggaagggtgttcttactgctcctttcaagctgctcaataatcgccttggagtgatcttcacctatgctgtgtacaagtatgagcttcctgcaaatgccaggccacaggagcgcattcaggctgctataggctatttaggtggcatatttgatattgaagctcttgttgataagttgcttcaccagctcgcgggcaagcaatccatcatggtgaacgtttacgatactactaatgagagaccaatcagtatgtatggttcgaatgatacaggcagtggcatgtgccaggtcagcacactgaacttcggtgacccttcaaggaagcatgtgatgcactgcaggttcattcagagcccaccttggccttggatggcaataacgacatcaattggaactctagtgattgctttactgattggatacatagttcatgctaccgcaaaacggattgccagagttgaagatgactttcaggagatgagtttgctcaagaagcgcgcagaggatgcagatattgccaagtcacagttcttggctactgtttctcatgagatcagaactccaatgaatggtgtcctagggatgctccaaatgctcatggatactgatttggacacaacgcaacaagactatgttagaaccgcacaagccagtggcaaaactttggtatccctcatcaatgaggttcttgatcaggcaaagatcgagtctggtaaacttgagcttgaggtggtaccctttgatcttagaacagtttgtgatgacattttgtctctcttctgtgggaaagcacaggagaaaggactggagttggcagtgtttgtttctgatcaagttccacaaacactaattggcgatcctggaagaataaggcaaatcattacaaatcttgttggaaactctataaaattcacagagaagggtcatatctacttgacggttcatgtcgttgaagagattatgcattgtctggaggttgagacgggaactcagtatacaaataccttaagtggctacccagtggcgaacaggaagcgcagttgggaaaactttcgactttttagtagggaactgaactcatccgagatgccttttgcacccattgcatcagactcaataagcttgatgatatcagttgaagatacgggtgctggaatcccgttcgatgctcaattccgtgtcttcacccctttcatgcaagtaggtccatccatcgctcgcatacacgggggcactggcattggattaagcatcagcaaatgcttggttggcctcatgaagggagagatcggatttgcaagtaaaccccaagttggttctactttcaccttcactgcagtgcttacgagagtccattccagtagaaatgagaataaatcatcagaatttaaagagatcaatgcattggtggtagatcatagaccggtccgtgccaaggttactaagtatcatttgcagagactgggagttcagactgaattgacaactgacctagatcagtatatttctaaaaataattgtggatcacagattgcaaagcttgtgctcattgacaaagaaacctggctgaaagaatcccactccatgcctcttttggttactaaattgaggaacaaagatcagccagactctacaaagttatttcttttggagaaccctaaaagttctgtcaagagcaattcacatatattcagggaatataacttgaatgtaattatgaagccacttcgtgcaagcatgcttcaggtttcgctacaaagagcattgggtgggatagataaactgcattgcaggaatggagtagtcggaaattcaacactgggcagccttcttcacaagaagcgaatcatagtggttgatgataatattgtaaatctcaaggttgctgcaggcgctctaaagaaatatggcgcggaagtaacttgtgctgacagtgggaaggaagcaatcacactgctgaaacctcctcacaattttgatgcttgtttcatggacatacagatgcctgaaatggatggatttgaagccacaaaaaggattagagtgatggaaagagatctaaatgagcaaatagaatgtggagaagccccaccagaatgcgtaggggttcggcagtggcgaactccaatattagccatgacagcagatgttatccaggcgacacatgaacaatgcttgaaatctgaaatggatggttatgtttccaagccattcgagggcgagcaattgtacagagaagtagctcgctttttcctaaatcaagaccaagttcagtag

SEQ ID NO: 5 - protein, 1007 aa, Zea mays
MTVTARGRSGGAGMGEKGGAPGLGFLSLDRMRVLLPPLRLPEKLSARTLRTHLFTNYRMRKVRDSSSWLIPLWVSSWVLVASLIYLCMSYQAVGKRRESLASMCDERARMLQDQFNVSMNHLQALAILVSTFHHSKTPSAIDQKTFARYAERTAFERPLTSGVAYGVRVTHTEREQFERQQGWSIKKMYSSKTKNQSQGPGNAEDAEVREPAEEYAPVIFAQDAYKHVISFDLLSGADDRDNVLRARESGKGVLTAPFKLLNNRLGVIFTYAVYKYELPANARPQERIQAAIGYLGGIFDIEALVDKLLHQLAGKQSIMVNVYDTTNERPISMYGSNDTGSGMCQVSTLNFGDPSRKHVMHCRFIQSPPWPWMAITTSIGTLVIALLIGYIVHATAKRIARVEDDFQEMSLLKKRAEDADIAKSQFLATVSHEIRTPMNGVLGMLQMLMDTDLDTTQQDYVRTAQASGKTLVSLINEVLDQAKIESGKLELEVVPFDLRTVCDDILSLFCGKAQEKGLELAVFVSDQVPQTLIGDPGRIRQIITNLVGNSIKFTEKGHIYLTVHVVEEIMHCLEVETGTQYTNTLSGYPVANRKRSWENFRLFSRELNSSEMPFAPIASDSISLMISVEDTGAGIPFDAQFRVFTPFMQVGPSIARIHGGTGIGLSISKCLVGLMKGEIGFASKPQVGSTFTFTAVLTRVHSSRNENKSSEFKEINALVVDHRPVRAKVTKYHLQRLGVQTELTTDLDQYISKNNCGSQIAKLVLIDKETWLKESHSMPLLVTKLRNKDQPDSTKLFLLENPKSSVKSNSHIFREYNLNVIMKPLRASMLQVSLQRALGGIDKLHCRNGVVGNSTLGSLLHKKRIIVVDDNIVNLKVAAGALKKYGAEVTCADSGKEAITLLKPPHNFDACFMDIQMPEMDGFEATKRIRVMERDLNEQIECGEAPPECVGVRQWRTPILAMTADVIQATHEQCLKSEMDGYVSKPFEGEQLYREVARFFLNQDQVQ

SEQ ID NO: 6 - DNA, 3021 bp, Zea mays
atgaccgttacggcgcgcgggaggagcggtggagccgggatgggggagaagggcggcgctcccgggctcggcttcctgagcctcgatcggatgcgcgtcctcctgccgccgctccgcctgccggagaagctgtcggccaggacgctgcggacgcacctcttcactaactatcggatgcgcaaggtgcgggacagctccagctggctcatcccgctctgggtcagctcttgggtcctcgtcgcctccctcatctacctctgcatgagctaccaggccgtcggcaagcgccgggagtctctcgccagcatgtgcgacgagcgcgcgcgcatgctccaggaccaatttaacgtcagcatgaaccacctccaggcgctcgccatcctcgtctccaccttccaccactccaagacaccctccgccatcgaccagaaaacgttcgcgaggtatgcggagaggacggcgttcgagcggccgctgacgagcggggtcgcgtacggggtgcgggtcacgcacaccgagcgggagcagttcgagcgccagcagggatggagcatcaagaagatgtactcctccaagaccaagaatcagtcgcagggacccgggaacgccgaggacgccgaggtgcgggagccggcggaggagtatgctcccgtaatcttcgcgcaggacgcctacaagcacgtcatctccttcgacttgctatccggcgctgatgaccgggacaacgtactccgagctagagaatctgggaagggtgttcttactgctcctttcaagctgctcaataatcgccttggagtgatcttcacctatgctgtgtacaagtatgagcttcctgcaaatgccaggccacaggagcgcattcaggctgctataggctatttaggtggcatatttgatattgaagctcttgttgataagttgcttcaccagctcgcgggcaagcaatccatcatggtgaacgtttacgatactactaatgagagaccaatcagtatgtatggttcgaatgatacaggcagtggcatgtgccaggtcagcacactgaacttcggtgacccttcaaggaagcatgtgatgcactgcaggttcattcagagcccaccttggccttggatggcaataacgacatcaattggaactctagtgattgctttactgattggatacatagttcatgctaccgcaaaacggattgccagagttgaagatgactttcaggagatgagtttgctcaagaagcgcgcagaggatgcagatattgccaagtcacagttcttggctactgtttctcatgagatcagaactccaatgaatggtgtcctagggatgctccaaatgctcatggatactgatttggacacaacgcaacaagactatgttagaaccgcacaagccagtggcaaaactttggtatccctcatcaatgaggttcttgatcaggcaaagatcgagtctggtaaacttgagcttgaggtggtaccctttgatcttagaacagtttgtgatgacattttgtctctcttctgtgggaaagcacaggagaaaggactggagttggcagtgtttgtttctgatcaagttccacaaacactaattggcgatcctggaagaataaggcaaatcattacaaatcttgttggaaactctataaaattcacagagaagggtcatatctacttgacggttcatgtcgttgaagagattatgcattgtctggaggttgagacgggaactcagtatacaaataccttaagtggctacccagtggcgaacaggaagcgcagttgggaaaactttcgactttttagtagggaactgaactcatccgagatgccttttgcacccattgcatcagactcaataagcttgatgatatcagttgaagatacgggtgctggaatcccgttcgatgctcaattccgtgtcttcacccctttcatgcaagtaggtccatccatcgctcgcatacacgggggcactggcattggattaagcatcagcaaatgcttggttggcctcatgaagggagagatcggatttgcaagtaaaccccaagttggttctactttcaccttcactgcagtgcttacgagagtccattccagtagaaatgagaataaatcatcagaatttaaagagatcaatgcattggtggtagatcatagaccggtccgtgccaaggttactaagtatcatttgcagagactgggagttcagactgaattgacaactgacctagatcagtatatttctaaaaataattgtggatcacagattgcaaagcttgtgctcattgacaaagaaacctggctgaaagaatcccactccatgcctcttttggttactaaattgaggaacaaagatcagccagactctacaaagttatttcttttggagaaccctaaaagttctgtcaagagcaattcacatatattcagggaatataacttgaatgtaattatgaagccacttcgtgcaagcatgcttcaggtttcgctacaaagagcattgggtgggatagataaactgcattgcaggaatggagtagtcggaaattcaacactgggcagccttcttcacaagaagcgaatcatagtggttgatgataatattgtaaatctcaaggttgctgcaggcgctctaaagaaatatggcgcggaagtaacttgtgctgacagtgggaaggaagcaatcacactgctgaaacctcctcacaattttgatgcttgtttcatggacatacagatgcctgaaatggatggatttgaagccacaaaaaggattagagtgatggaaagagatctaaatgagcaaatagaatgtggagaagccccaccagaatgcgtaggggttcggcagtggcgaactccaatattagccatgacagcagatgttatccaggcgacacatgaacaatgcttgaaatctgaaatggatggttatgtttccaagccattcgagggcgagcaattgtacagagaagtagctcgctttttcctaaatcaagaccaagttcag

SEQ ID NO: 7 - DNA, 2898 bp, Zea mays
  CDS (1)...(2895): ZmCKI2
atgggggacgactacctagctgaaactgaacctgaggatgaggttgcacaatcaatgtggcctgagaacatagtagacaaacaccagaggcagttcagaatggaaaagtttaggaaggaccaagatgctttcaaggatgtgaagtttgatgaaaagcctgttcacgtggactttcagcggcttatggaaatggcaaattctgagaaaggtgtttctcagatgcaatactttatgaagcactgggaatataaaagagccaatgctgctcggcttctggaggaagagcttggtcttctctgccaacaaagaaaagagattgaacaaaacaagcaagaaatcttggaggagcaacgctttcaggatgaaagctattatgctgtaaagcggcatgtaccaatactggatgaagtttatgaagatgaatggaagcgaccgagcaaaaagaatgatgatctctctcatagtagagagactaaaatagacgcagatcatgacagtgtttcctactggaaagagagggcaactaagttagaggaaaaacttgaggaaagcatccaaagggagcgttctttggttgagaaattggaggaaaatataaagaatctgcagtcacatacaccagctgaagaattctctggaatgctgaaacgtgctgattacttcttgcatttggttctacaaagcgctcctattgttattgctcatcaggatgctgatttacggtaccgtttcatatttaaccactatccaacacttgctgatgaggatgttattggtaagaccgactacgagatattatccggtgagggtatagaagagatgaatagtgtcaagcgagaagttatggccacaggaatggcaactaaaagagaattcgcgtttaatacaccaatgtttggggcaaagacatttgtgacatacattgagccagttttcagcaaaggtggggaagcaattggtgtgaattatgttgcaatggatataacagatcaggtcaaaagaagagagaaaatggcagacataagggtgagggaagctgtacaaaatgccaaggaaactgaacttagcaaatctcttcacataacagaggaaacaatgcgagcaaaacaaatgctagctaccatgtcccatgagatcagatctcctctttcaggtgttcttagcatggctgaaattcttgcaactaccaaactggataaagaacagcgtcagttgctagaagttatgttatcctctggagatcttgtattgcagttgatcaatgacatccttgatctttccaaagtggagtcaggggctatgaaactagaatctacgacatttagaccaagggaagtagttaaacatgtactgcaaactgccgcggcttctctgaagaaggaattgacccttgaaggttgcataggtgacgatgttccagcggaggttattggcgatgtgctaaggattcgtcaaattctgaccaacttgatcagcaatgcagtcaaatttacacatgaagggaaagttggaataaatcttcaggtcgtgcatgacaagcaaccggtatgcaaaatagagagcgggaaaattcataagcgaacgttttctgggacagcaattactgctgcagaaactgctgctgtctctacaagaaactgtgataaagatactttgcattgcttgaagcatgaagatgcttttcagaatggtgttccaacatgtgaaaaattcacggatactgagggagatgaagttgtttggcttcgctgtgatgtatatgacactggaataggaataccagagaagtccttaccattgctgttcaagaagtatatgcaagctagcgccgatcatgcgaggaagtatggtgggacagggcttggcctagcaatctgcaagcagttggttgagttgatgggtggtactctgactgtggtcagcaaagagagcgaaggatcaacatttacatttgtgctgccttgtaaaatccctgaaaaagaggagcatagtgatgatcccgatgaagtgcacagttgtcagaatgattccgcaaacagtgatatagaaggttctttcttttttaagccacaaatgcgggcttcccttctgtctcctggactttcaataatgaacaacacaaagttattcagtaccaagcttatgtgctatgacccgcctgacataccagatgaccgcaaattgttcttaaatggtttctcatcagcagaacataattttacaaatacttctactactcaacaaccgaatggtgctagtgttaggagcacagctgaagaagaacatgataatgcaatggtccttgaactgaatagtcaagctgaacgggtttccagttcccgaggagatttggtatccgtttcaggtgctgctccatgcaaagttcctgaagaacaatcactccacaagaaatcaaaatgttctccaaccagcaacaaagcaaagatcctcctagttgaagataacaaggtcaacataatagtggcaaaatcaatgttgcagcagctgggccatggaatagatatcgtaaataatgggatgcaagcaatccgtgcagtacagcagcatcaatatgatcttattctgatggatgttcacatgccagaaatggatggcctgcaagccactaaacttatccggtcttttgagaataccggctgttgggatgtttctgtaaagcctgaagataatccgatgataactgactcagctatttcatcagattctgcacatgcaaaaaagcaagggcagagggttcctataatcgcgatgacagcgaattcattcgcagagagtgccgaggaatgccttgctgcaggcatggattcctacatatctaagccggtgaacttccagaatataaaagagtgcctgcggcggtacttggtgtctcagtga

SEQ ID NO: 8 - protein, 965 aa, Zea mays
MGDDYLAETEPEDEVAQSMWPENIVDKHQRQFRMEKFRKDQDAFKDVKFDEKPVHVDFQRLMEMANSEKGVSQMQYFMKHWEYKRANAARLLEEELGLLCQQRKEIEQNKQEILEEQRFQDESYYAVKRHVPILDEVYEDEWKRPSKKNDDLSHSRETKIDADHDSVSYWKERATKLEEKLEESIQRERSLVEKLEENIKNLQSHTPAEEFSGMLKRADYFLHLVLQSAPIVIAHQDADLRYRFIFNHYPTLADEDVIGKTDYEILSGEGIEEMNSVKREVMATGMATKREFAFNTPMFGAKTFVTYIEPVFSKGGEAIGVNYVAMDITDQVKRREKMADIRVREAVQNAKETELSKSLHITEETMRAKQMLATMSHEIRSPLSGVLSMAEILATTKLDKEQRQLLEVMLSSGDLVLQLINDILDLSKVESGAMKLESTTFRPREVVKHVLQTAAASLKKELTLEGCIGDDVPAEVIGDVLRIRQILTNLISNAVKFTHEGKVGINLQVVHDKQPVCKIESGKIHKRTFSGTAITAAETAAVSTRNCDKDTLHCLKHEDAFQNGVPTCEKFTDTEGDEVVWLRCDVYDTGIGIPEKSLPLLFKKYMQASADHARKYGGTGLGLAICKQLVELMGGTLTVVSKESEGSTFTFVLPCKIPEKEEHSDDPDEVHSCQNDSANSDIEGSFFFKPQMRASLLSPGLSIMNNTKLFSTKLMCYDPPDIPDDRKLFLNGFSSAEHNFTNTSTTQQPNGASVRSTAEEEHDNAMVLELNSQAERVSSSRGDLVSVSGAAPCKVPEEQSLHKKSKCSPTSNKAKILLVEDNKVNIIVAKSMLQQLGHGIDIVNNGMQAIRAVQQHQYDLILMDVHMPEMDGLQATKLIRSFENTGCWDVSVKPEDNPMITDSAISSDSAHAKKQGQRVPIIAMTANSFAESAEECLAAGMDSYISKPVNFQNIKECLRRYLVSQ

SEQ ID NO: 9 - DNA, 2895 bp, Zea mays
atgggggacgactacctagctgaaactgaacctgaggatgaggttgcacaatcaatgtggcctgagaacatagtagacaaacaccagaggcagttcagaatggaaaagtttaggaaggaccaagatgctttcaaggatgtgaagtttgatgaaaagcctgttcacgtggactttcagcggcttatggaaatggcaaattctgagaaaggtgtttctcagatgcaatactttatgaagcactgggaatataaaagagccaatgctgctcggcttctggaggaagagcttggtcttctctgccaacaaagaaaagagattgaacaaaacaagcaagaaatcttggaggagcaacgctttcaggatgaaagctattatgctgtaaagcggcatgtaccaatactggatgaagtttatgaagatgaatggaagcgaccgagcaaaaagaatgatgatctctctcatagtagagagactaaaatagacgcagatcatgacagtgtttcctactggaaagagagggcaactaagttagaggaaaaacttgaggaaagcatccaaagggagcgttctttggttgagaaattggaggaaaatataaagaatctgcagtcacatacaccagctgaagaattctctggaatgctgaaacgtgctgattacttcttgcatttggttctacaaagcgctcctattgttattgctcatcaggatgctgatttacggtaccgtttcatatttaaccactatccaacacttgctgatgaggatgttattggtaagaccgactacgagatattatccggtgagggtatagaagagatgaatagtgtcaagcgagaagttatggccacaggaatggcaactaaaagagaattcgcgtttaatacaccaatgtttggggcaaagacatttgtgacatacattgagccagttttcagcaaaggtggggaagcaattggtgtgaattatgttgcaatggatataacagatcaggtcaaaagaagagagaaaatggcagacataagggtgagggaagctgtacaaaatgccaaggaaactgaacttagcaaatctcttcacataacagaggaaacaatgcgagcaaaacaaatgctagctaccatgtcccatgagatcagatctcctctttcaggtgttcttagcatggctgaaattcttgcaactaccaaactggataaagaacagcgtcagttgctagaagttatgttatcctctggagatcttgtattgcagttgatcaatgacatccttgatctttccaaagtggagtcaggggctatgaaactagaatctacgacatttagaccaagggaagtagttaaacatgtactgcaaactgccgcggcttctctgaagaaggaattgacccttgaaggttgcataggtgacgatgttccagcggaggttattggcgatgtgctaaggattcgtcaaattctgaccaacttgatcagcaatgcagtcaaatttacacatgaagggaaagttggaataaatcttcaggtcgtgcatgacaagcaaccggtatgcaaaatagagagcgggaaaattcataagcgaacgttttctgggacagcaattactgctgcagaaactgctgctgtctctacaagaaactgtgataaagatactttgcattgcttgaagcatgaagatgcttttcagaatggtgttccaacatgtgaaaaattcacggatactgagggagatgaagttgtttggcttcgctgtgatgtatatgacactggaataggaataccagagaagtccttaccattgctgttcaagaagtatatgcaagctagcgccgatcatgcgaggaagtatggtgggacagggcttggcctagcaatctgcaagcagttggttgagttgatgggtggtactctgactgtggtcagcaaagagagcgaaggatcaacatttacatttgtgctgccttgtaaaatccctgaaaaagaggagcatagtgatgatcccgatgaagtgcacagttgtcagaatgattccgcaaacagtgatatagaaggttctttcttttttaagccacaaatgcgggcttcccttctgtctcctggactttcaataatgaacaacacaaagttattcagtaccaagcttatgtgctatgacccgcctgacataccagatgaccgcaaattgttcttaaatggtttctcatcagcagaacataattttacaaatacttctactactcaacaaccgaatggtgctagtgttaggagcacagctgaagaagaacatgataatgcaatggtccttgaactgaatagtcaagctgaacgggtttccagttcccgaggagatttggtatccgtttcaggtgctgctccatgcaaagttcctgaagaacaatcactccacaagaaatcaaaatgttctccaaccagcaacaaagcaaagatcctcctagttgaagataacaaggtcaacataatagtggcaaaatcaatgttgcagcagctgggccatggaatagatatcgtaaataatgggatgcaagcaatccgtgcagtacagcagcatcaatatgatcttattctgatggatgttcacatgccagaaatggatggcctgcaagccactaaacttatccggtcttttgagaataccggctgttgggatgtttctgtaaagcctgaagataatccgatgataactgactcagctatttcatcagattctgcacatgcaaaaaagcaagggcagagggttcctataatcgcgatgacagcgaattcattcgcagagagtgccgaggaatgccttgctgcaggcatggattcctacatatctaagccggtgaacttccagaatataaaagagtgcctgcggcggtacttggtgtctcag

SEQ ID NO: 10 - DNA, 2482 bp, Zea mays
  CDS (3)...(1790): ZmCRE1 partial
cccacgcgtccggaatgcttgatatgctgttagacactgacctaacctcaactcagagggattttgcacaaaccgctcaagtctgtggaaaggctttaatatcgctaatcaatgaagtgcttgacagagcgaaaattgaagccagaaagttggatcttgaatctgtaccatttgatctgagatccatccttgatgatgtcatctcattattctcttcaaagtcaagagagaagggaattgagcttgatgtatatgtctctgaaagagttcccgaaatcttgttgggtgatcctggaaggtttcgacagataattacaaatttagtgggcaactcaattaagttcacagaacggggacatatttttgtacaagttcatctggcagatcattcaaatcttgcaacagaatccaaagttgagccggtggctaatgggatgaatggacatacagatgagaaaactgctgtagcaaccagtgtttctctcaacacactaagtggttttgaagctgctgatagccgaaatagttgggaaaacttcaagcttttgctttcttatgagaaaaatgagatgccctatgaaagtgtatctgataaagttactcttgtagtaagtgtggaagatacaggaataggtataccattggatgcacaagccaaggtgttcacacctttcatgcaggctgatagttcgacttccaggacatatggtggtactggaattggattgagcatcagcaaatgtcttgttgaactaatgggtggtcagataaactttgtcagccgaccacatgttgggagtacgttcacatttactgcggttctcaaaagatgtgacagaagcgctattggtgacagtaagcctgttatgttgcatcctcttccatccagtttcaaaggtttatctgcactattggttgatagaagaccagtaagagctactgtaactaagtatcacttgcaaaggctgggaattgcctgtgatgttgttgctaccattgaactggctcttggtgtgctgtccgggagaaatggcagttccctaaccagtatgaagcaaccatgcatgttattgatcgagagtgattcatggggcttcaatattgatgtttctttacgatctagactcctggagatgaagcagaatggtcacacacatgtattacccaaaatcatccttcttgcagctgcagaatcgggcaaactcaaagaacactatgcagttgattctgtgatcacgaaacctctgaaagcaagcgcacttgccgcttgtctattccaaacacttggtatcaaacagtcaagcaacgagagaagtgacaactcaggttctctttacgggttgcttcttggcaagaacatattggtggttgatgacaacaaggtaaatcttagagtggctgctggcacgttaaagaaatttggagcaaaggtggagtgcgtggagagtggaaaagatgccctcgccctcctacaggtcccatataagtttcatctttgtctcatggacattcagatgcccgaaatggatgggtttgaggctaccaagcaaataagggcaatggaagcgaaggcaaatgagcaggcagtcgcctgtgacgattcagaaatagatggcgcgacaagggcagcaagatggcgcctgcctgttcttgcaatgaccgccgatgtcatccaggccacccatgaagagtgcacgaagtgcgggatggatgggtacgtcacaaagcccttcgaggagaagcagctcttccaggcgttgcagaagttcttggaccccggcatgtcctgctaacgccgaagtgatgcgttggaggtgaggcactattctcctctattctgtaattccctaaccttggccgcaacctgatccccaagaaagaccgtgcagcgcgggctgctcgtcgatcatgccagcggggaacagcaggcagacaaggcagcacgcaccacgggccttcgctggtctgcctgcctgtgcaggcgtcgtgtaaatgtattgtatttgcagggatactgtttacagcgctagtatgccccgtgccacaatgtccgagctacctgtggctgatgcacgttgcgcataggtggtttttttttgtacagcttccaccatttttgtgctccagatcagaccaccgaaggaggcaagcatgctcgctggtgcagtgcagcactggaaggaaaacctgacttcctgagagtagatttttgctgtttaattgcattgggatttatgatgtattataatcagataaatgtgtgatgtaagctggcgtaattggcccaatgcatttccattgttatggatgagttgaaatggcattggttagactacactattgggtgtatgcccttccacagttccattttccctgcaaaatatcaaaatgtgcaaagggatagtggaatcatggcttgaattatatgaattttctttggtgttttaaaaaaaaaaaaaaaaaaaaaaaaaaaaaaaaaaaa

SEQ ID NO: 11 - protein, 596 aa, Zea mays
HASGMLDMLLDTDLTSTQRDFAQTAQVCGKALISLINEVLDRAKIEARKLDLESVPFDLRSILDDVISLFSSKSREKGIELDVYVSERVPEILLGDPGRFRQIITNLVGNSIKFTERGHIFVQVHLADHSNLATESKVEPVANGMNGHTDEKTAVATSVSLNTLSGFEAADSRNSWENFKLLLSYEKNEMPYESVSDKVTLVVSVEDTGIGIPLDAQAKVFTPFMQADSSTSRTYGGTGIGLSISKCLVELMGGQINFVSRPHVGSTFTFTAVLKRCDRSAIGDSKPVMLHPLPSSFKGLSALLVDRRPVRATVTKYHLQRLGIACDVVATIELALGVLSGRNGSSLTSMKQPCMLLIESDSWGFNIDVSLRSRLLEMKQNGHTHVLPKIILLAAAESGKLKEHYAVDSVITKPLKASALAACLFQTLGIKQSSNERSDNSGSLYGLLLGKNILVVDDNKVNLRVAAGTLKKFGAKVECVESGKDALALLQVPYKFHLCLMDIQMPEMDGFEATKQIRAMEAKANEQAVACDDSEIDGATRAARWRLPVLAMTADVIQATHEECTKCGMDGYVTKPFEEKQLFQALQKFLDPGMSC

SEQ ID NO: 12 - DNA, 1788 bp, Zea mays
cacgcgtccggaatgcttgatatgctgttagacactgacctaacctcaactcagagggattttgcacaaaccgctcaagtctgtggaaaggctttaatatcgctaatcaatgaagtgcttgacagagcgaaaattgaagccagaaagttggatcttgaatctgtaccatttgatctgagatccatccttgatgatgtcatctcattattctcttcaaagtcaagagagaagggaattgagcttgatgtatatgtctctgaaagagttcccgaaatcttgttgggtgatcctggaaggtttcgacagataattacaaatttagtgggcaactcaattaagttcacagaacggggacatatttttgtacaagttcatctggcagatcattcaaatcttgcaacagaatccaaagttgagccggtggctaatgggatgaatggacatacagatgagaaaactgctgtagcaaccagtgtttctctcaacacactaagtggttttgaagctgctgatagccgaaatagttgggaaaacttcaagcttttgctttcttatgagaaaaatgagatgccctatgaaagtgtatctgataaagttactcttgtagtaagtgtggaagatacaggaataggtataccattggatgcacaagccaaggtgttcacacctttcatgcaggctgatagttcgacttccaggacatatggtggtactggaattggattgagcatcagcaaatgtcttgttgaactaatgggtggtcagataaactttgtcagccgaccacatgttgggagtacgttcacatttactgcggttctcaaaagatgtgacagaagcgctattggtgacagtaagcctgttatgttgcatcctcttccatccagtttcaaaggtttatctgcactattggttgatagaagaccagtaagagctactgtaactaagtatcacttgcaaaggctgggaattgcctgtgatgttgttgctaccattgaactggctcttggtgtgctgtccgggagaaatggcagttccctaaccagtatgaagcaaccatgcatgttattgatcgagagtgattcatggggcttcaatattgatgtttctttacgatctagactcctggagatgaagcagaatggtcacacacatgtattacccaaaatcatccttcttgcagctgcagaatcgggcaaactcaaagaacactatgcagttgattctgtgatcacgaaacctctgaaagcaagcgcacttgccgcttgtctattccaaacacttggtatcaaacagtcaagcaacgagagaagtgacaactcaggttctctttacgggttgcttcttggcaagaacatattggtggttgatgacaacaaggtaaatcttagagtggctgctggcacgttaaagaaatttggagcaaaggtggagtgcgtggagagtggaaaagatgccctcgccctcctacaggtcccatataagtttcatctttgtctcatggacattcagatgcccgaaatggatgggtttgaggctaccaagcaaataagggcaatggaagcgaaggcaaatgagcaggcagtcgcctgtgacgattcagaaatagatggcgcgacaagggcagcaagatggcgcctgcctgttcttgcaatgaccgccgatgtcatccaggccacccatgaagagtgcacgaagtgcgggatggatgggtacgtcacaaagcccttcgaggagaagcagctcttccaggcgttgcagaagttcttggaccccggcatgtcctgc

SEQ ID NO: 13 - DNA, 3291 bp, Arabidopsis thaliana
  CDS (378)...(3146): AtCKI2
tgaatataatatacattgacctctctgtctcatgagcaagcacatgtcctctctctctctctctttctcttctccgtaaaaaaaattaccaatctttcacaattcattcacatgctcctctctctttcttcattggctattctcttcccaatgctcatttgaagtttatttactctactcccgacgaccacgactcttcttcagatctactcactccttctttctctccttcttcttcttcatttttccggtgaccggagtcggagaaggttctttattcagatcaaggttctggcttaaagaaaaaagttgtttgaattttgagatttgtctggtccattgtgttgctgttgttgtatgaagagaaaccttgatcaatggtctgtgaaatggagactgatcagattgaggaaatggatgtcgaagttttgtcttcgatgtggcccgaagatgttggaactgaagctgacaaacagttcaacgtcgagaaacctgccggagatttagacacgttgaaagaagttactatcgagacacggaccattgcggatatgacacggttaccaaacctattgaattcgactcatcaaggctcctctcaactaaccaaccttgtgaaacaatgggagtatatgcaagacaacgcggttcggctgttaaaagaagagctaaaaaatctcgatagacagagagaagaagccgaggctaaagagttgaagatcattgaggagtataagtttgagagcaacgagcctgagaatgttccggttttggatgagacgagtgatttgttccgcaggtttaggcagaaaaaacgagatgccttggtcgatagcaagaagattgagatctatgaggagtttgacactgttgcatattggaaacagaaggcgttgagtcttgagaaaatgcttgaggcgagtactgagagagaaaggcgattgatggagaagctgagtgagagtttgaaaactatggagagtcagtcagcaccggtccaagagcttactcagaatcttaagagagctgaaggtttcttgcatttcatacttcagaatgcacctattgttatgggccatcaggataaagatttacgctacttgttcatctacaacaagtatcctagtttacgggaacaggacattttgggaaaaacagacgtggagatattccatggaggtggagttaaagaatctgaagatttcaagagagaagttcttgagaaaggaaaagcttcaaagagagagatcacatttactacagatttatttggatcaaagacgtttttgatatatgttgagcctgtttacaacaaagctggcgagaaaatcggtataaactacatgggaatggaagtaactgatcaggtagtgaaaagggagaaaatggcgaaacttagagaagataacgctgtgagaaaggcgatggaatcagaactgaacaagactattcacattacagaggagacaatgagagcaaagcagatgctagcgacgatgtctcatgagataaggtcaccattgtcaggagtagtgggaatggctgagatactttcaactacaaaactggataaagagcaaagacagttgttgaatgtcatgatctcttctggtgatttggtgcttcagctaatcaacgacattcttgatctctccaaggttgaatcaggtgtgatgagattagaagctacaaagtttcgaccaagagaagtagtgaagcatgtgctacagacagctgcagcatcgctgaagaaatctttgacattagaaggaaacattgcagatgatgttcctattgaggtagttggagatgttctaaggattaggcagatcctcaccaatttgataagcaatgctatcaagtttacacatgaaggaaatgtaggaatcaaactccaagtgatatcagaaccatcctttgtgcgggataacgcattgaacgcagacaccgaggaacacgaacaaaacggtttgaccgagacttcagtttggatttgctgtgacgtatgggacactggaattggaatcccagaaaacgctcttccatgtttgttcaagaagtacatgcaagcaagcgctgatcatgcccgaaaatacggtgggactggtctcggacttgctatttgtaaacagctggttgagttaatgggaggccaactcactgtgacaagccgggtgagcgaaggttcaacgttcacatttatattaccctacaaagttggaagatcagatgattattcagatgatcaagatgagttctctgatatggcggatcaacaatctgaaccagacgatacagctgaaggatatttccagtttaaaccgctcttaggatcgatatattcgaatggcggaccggggatcagcaatgacttcttacctcataaagtcatgcttactagtcctattaagctcatcaatggttttgtcgctgatccctctaataacactggacagagcgagatgctacagcttgaaaacggtggttacatggatgaatctaaactcgaaaccagttctggtcattgccctgaatcagctcaccaatatgagaatggaaatggtcgatgtttctctaaggaatctgaatcttgtagcagttcacaagctagctcagaaggtggaaccttagaaatggagtcagagctcacagtttcatctcatagggaagaggaaaaagccgagacagaagtaaaagaaacatcaaagccaaagattttgcttgtggaagataataagatcaacatcatggttgcaaagtcgatgatgaagcagttaggccataccatggatattgctaataatggagttgaagccataaccgcgattaatagctctagctacgatctggtactcatggatgtgtgcatgccggtgctcgatggtttaaaagctacaagactgatccgttcgtatgaagaaactgggaactggaatgctgcaatagaagccggagtagatatatcgacatcggagaatgaacaagtttgtatgcgtcccacaaaccggctgcctataatcgcgatgacggcaaatactttagcagagagttcagaagaatgttatgcaaatggtatggactcgtttatttcgaaacctgtaacgttgcaaaaactgagagagtgtttgcaacagtatttgcactgagatttcagatttttgtgttttgtagattaagaaatggttgtgttgtatataaattgtgtaggaaaaaagttttggagagctactaagtagcttcctctttttagagatgtatagttcaataaataaataaaagcttctcttcttt

SEQ ID NO: 14 - protein, 922 aa, Arabidopsis thaliana
MVCEMETDQIEEMDVEVLSSMWPEDVGTEADKQFNVEKPAGDLDTLKEVTIETRTIADMTRLPNLLNSTHQGSSQLTNLVKQWEYMQDNAVRLLKEELKNLDRQREEAEAKELKIIEEYKFESNEPENVPVLDETSDLFRRFRQKKRDALVDSKKIEIYEEFDTVAYWKQKALSLEKMLEASTERERRLMEKLSESLKTMESQSAPVQELTQNLKRAEGFLHFILQNAPIVMGHQDKDLRYLFIYNKYPSLREQDILGKTDVEIFHGGGVKESEDFKREVLEKGKASKREITFTTDLFGSKTFLIYVEPVYNKAGEKIGINYMGMEVTDQVVKREKMAKLREDNAVRKAMESELNKTIHITEETMRAKQMLATMSHEIRSPLSGVVGMAEILSTTKLDKEQRQLLNVMISSGDLVLQLINDILDLSKVESGVMRLEATKFRPREVVKHVLQTAAASLKKSLTLEGNIADDVPIEVVGDVLRIRQILTNLISNAIKFTHEGNVGIKLQVISEPSFVRDNALNADTEEHEQNGLTETSVWICCDVWDTGIGIPENALPCLFKKYMQASADHARKYGGTGLGLAICKQLVELMGGQLTVTSRVSEGSTFTFILPYKVGRSDDYSDDQDEFSDMADQQSEPDDTAEGYFQFKPLLGSIYSNGGPGISNDFLPHKVMLTSPIKLINGFVADPSNNTGQSEMLQLENGGYMDESKLETSSGHCPESAHQYENGNGRCFSKESESCSSSQASSEGGTLEMESELTVSSHREEEKAETEVKETSKPKILLVEDNKINIMVAKSMMKQLGHTMDIANNGVEAITAINSSSYDLVLMDVCMPVLDGLKATRLIRSYEETGNWNAAIEAGVDISTSENEQVCMRPTNRLPIIAMTANTLAESSEECYANGMDSFISKPVTLQKLRECLQQYLH

SEQ ID NO: 15 - DNA, 2769 bp, Arabidopsis thaliana
atggtctgtgaaatggagactgatcagattgaggaaatggatgtcgaagttttgtcttcgatgtggcccgaagatgttggaactgaagctgacaaacagttcaacgtcgagaaacctgccggagatttagacacgttgaaagaagttactatcgagacacggaccattgcggatatgacacggttaccaaacctattgaattcgactcatcaaggctcctctcaactaaccaaccttgtgaaacaatgggagtatatgcaagacaacgcggttcggctgttaaaagaagagctaaaaaatctcgatagacagagagaagaagccgaggctaaagagttgaagatcattgaggagtataagtttgagagcaacgagcctgagaatgttccggttttggatgagacgagtgatttgttccgcaggtttaggcagaaaaaacgagatgccttggtcgatagcaagaagattgagatctatgaggagtttgacactgttgcatattggaaacagaaggcgttgagtcttgagaaaatgcttgaggcgagtactgagagagaaaggcgattgatggagaagctgagtgagagtttgaaaactatggagagtcagtcagcaccggtccaagagcttactcagaatcttaagagagctgaaggtttcttgcatttcatacttcagaatgcacctattgttatgggccatcaggataaagatttacgctacttgttcatctacaacaagtatcctagtttacgggaacaggacattttgggaaaaacagacgtggagatattccatggaggtggagttaaagaatctgaagatttcaagagagaagttcttgagaaaggaaaagcttcaaagagagagatcacatttactacagatttatttggatcaaagacgtttttgatatatgttgagcctgtttacaacaaagctggcgagaaaatcggtataaactacatgggaatggaagtaactgatcaggtagtgaaaagggagaaaatggcgaaacttagagaagataacgctgtgagaaaggcgatggaatcagaactgaacaagactattcacattacagaggagacaatgagagcaaagcagatgctagcgacgatgtctcatgagataaggtcaccattgtcaggagtagtgggaatggctgagatactttcaactacaaaactggataaagagcaaagacagttgttgaatgtcatgatctcttctggtgatttggtgcttcagctaatcaacgacattcttgatctctccaaggttgaatcaggtgtgatgagattagaagctacaaagtttcgaccaagagaagtagtgaagcatgtgctacagacagctgcagcatcgctgaagaaatctttgacattagaaggaaacattgcagatgatgttcctattgaggtagttggagatgttctaaggattaggcagatcctcaccaatttgataagcaatgctatcaagtttacacatgaaggaaatgtaggaatcaaactccaagtgatatcagaaccatcctttgtgcgggataacgcattgaacgcagacaccgaggaacacgaacaaaacggtttgaccgagacttcagtttggatttgctgtgacgtatgggacactggaattggaatcccagaaaacgctcttccatgtttgttcaagaagtacatgcaagcaagcgctgatcatgcccgaaaatacggtgggactggtctcggacttgctatttgtaaacagctggttgagttaatgggaggccaactcactgtgacaagccgggtgagcgaaggttcaacgttcacatttatattaccctacaaagttggaagatcagatgattattcagatgatcaagatgagttctctgatatggcggatcaacaatctgaaccagacgatacagctgaaggatatttccagtttaaaccgctcttaggatcgatatattcgaatggcggaccggggatcagcaatgacttcttacctcataaagtcatgcttactagtcctattaagctcatcaatggttttgtcgctgatccctctaataacactggacagagcgagatgctacagcttgaaaacggtggttacatggatgaatctaaactcgaaaccagttctggtcattgccctgaatcagctcaccaatatgagaatggaaatggtcgatgtttctctaaggaatctgaatcttgtagcagttcacaagctagctcagaaggtggaaccttagaaatggagtcagagctcacagtttcatctcatagggaagaggaaaaagccgagacagaagtaaaagaaacatcaaagccaaagattttgcttgtggaagataataagatcaacatcatggttgcaaagtcgatgatgaagcagttaggccataccatggatattgctaataatggagttgaagccataaccgcgattaatagctctagctacgatctggtactcatggatgtgtgcatgccggtgctcgatggtttaaaagctacaagactgatccgttcgtatgaagaaactgggaactggaatgctgcaatagaagccggagtagatatatcgacatcggagaatgaacaagtttgtatgcgtcccacaaaccggctgcctataatcgcgatgacggcaaatactttagcagagagttcagaagaatgttatgcaaatggtatggactcgtttatttcgaaacctgtaacgttgcaaaaactgagagagtgtttgcaacagtatttgcactga

SEQ ID NO: 16 - DNA, 1431 bp, Arabidopsis thaliana
  CDS (1)...(1428): AtAPK3
atggctacaaagttagagtcttttaaggtggtggagaaattgggtgttgagaaaggtgaaaaagggaagatgatgagtaaaaaaaagaatgttaagaaagatggagatgagagtgaaagtgggttttggtttagattcaagtttatctttagttgtatctcttctagatcaaaagttgatagttccatgaatgctactgctgttattgcagaacctaagaaagttattgagaagcttgaaggtcatccagctccgactaaagacactggctgtgcggagagcggttcctcaacacctctaatgagcggggaattgaagtattcttctaagctacggattttcatgtttaacgaccttaagttagccactaggaatttcagaccagagtctcttctcggtgagggtgggtttggatgtgtttttaaaggatggatagaggagaatggaactgcacctgtcaagcctggtactggtctaactgtagctgtcaaaacactgaacccagatggccttcaaggtcacaaggagtggctggctgaaattaacttccttggaaaccttgttcaccccagtctggttaaattagttggctactgcatggaagaagaccaaaggctgcttgtttacgagtttatgccacgaggaagtttggagaaccatcttttcagaaggaccttacctctcccttggtctgttcgaatgaaaattgcacttggtgcagctaaaggtctcgcctttcttcacgaagaagctgagaagccagtcatatatcgagatttcaaaacatctaatattttactggatggggaatataatgcaaagctctctgattttgggcttgcaaaagatgctccagacgagaaaaaatcccacgtatcaacccgagtcatgggaacttatggttatgctgcccctgaatatgtaatgactggacatctgacaacgaagagcgatgtatacagttttggagtagttttacttgaaatattaactggacgaagatctgtggataaaagtcggccgaacggggaacagaacttggtagaatgggtgagacctcatctcttagacaagaaaaggttttaccggctattagatcctcggttagagggtcactactcgatcaaaggtgctcagaaagccacacaagtagcggcgcaatgccttaaccgagactccaaagccagaccgaaaatgagtgaagttgtggaagccttaaagccattaccaaatcttaaagactttgctagctcttcttcttctttccaaacaatgcagcctgttgctaagaacggagtcagaacacaaggaggagggtttgtgtcgagaaatggaccgccaatgaggagtttgtctagcttaaacttgcctcaagcatccccttatcgttatgcccgtcaatcaccaaagcctaaaggaaaagagccatga

SEQ ID NO: 17 - protein, 476 aa, Arabidopsis thaliana
MATKLESFKVVEKLGVEKGEKGKMMSKKKNVKKDGDESESGFWFRFKFIFSCISSRSKVDSSMNATAVIAEPKKVIEKLEGHPAPTKDTGCAESGSSTPLMSGELKYSSKLRIFMFNDLKLATRNFRPESLLGEGGFGCVFKGWIEENGTAPVKPGTGLTVAVKTLNPDGLQGHKEWLAEINFLGNLVHPSLVKLVGYCMEEDQRLLVYEFMPRGSLENHLFRRTLPLPWSVRMKIALGAAKGLAFLHEEAEKPVIYRDFKTSNILLDGEYNAKLSDFGLAKDAPDEKKSHVSTRVMGTYGYAAPEYVMTGHLTTKSDVYSFGVVLLEILTGRRSVDKSRPNGEQNLVEWVRPHLLDKKRFYRLLDPRLEGHYSIKGAQKATQVAAQCLNRDSKARPKMSEVVEALKPLPNLKDFASSSSSFQTMQPVAKNGVRTQGGGFVSRNGPPMRSLSSLNLPQASPYRYARQSPKPKGKEP

SEQ ID NO: 18 - DNA, 1428 bp, Arabidopsis thaliana
atggctacaaagttagagtcttttaaggtggtggagaaattgggtgttgagaaaggtgaaaaagggaagatgatgagtaaaaaaaagaatgttaagaaagatggagatgagagtgaaagtgggttttggtttagattcaagtttatctttagttgtatctcttctagatcaaaagttgatagttccatgaatgctactgctgttattgcagaacctaagaaagttattgagaagcttgaaggtcatccagctccgactaaagacactggctgtgcggagagcggttcctcaacacctctaatgagcggggaattgaagtattcttctaagctacggattttcatgtttaacgaccttaagttagccactaggaatttcagaccagagtctcttctcggtgagggtgggtttggatgtgtttttaaaggatggatagaggagaatggaactgcacctgtcaagcctggtactggtctaactgtagctgtcaaaacactgaacccagatggccttcaaggtcacaaggagtggctggctgaaattaacttccttggaaaccttgttcaccccagtctggttaaattagttggctactgcatggaagaagaccaaaggctgcttgtttacgagtttatgccacgaggaagtttggagaaccatcttttcagaaggaccttacctctcccttggtctgttcgaatgaaaattgcacttggtgcagctaaaggtctcgcctttcttcacgaagaagctgagaagccagtcatatatcgagatttcaaaacatctaatattttactggatggggaatataatgcaaagctctctgattttgggcttgcaaaagatgctccagacgagaaaaaatcccacgtatcaacccgagtcatgggaacttatggttatgctgcccctgaatatgtaatgactggacatctgacaacgaagagcgatgtatacagttttggagtagttttacttgaaatattaactggacgaagatctgtggataaaagtcggccgaacggggaacagaacttggtagaatgggtgagacctcatctcttagacaagaaaaggttttaccggctattagatcctcggttagagggtcactactcgatcaaaggtgctcagaaagccacacaagtagcggcgcaatgccttaaccgagactccaaagccagaccgaaaatgagtgaagttgtggaagccttaaagccattaccaaatcttaaagactttgctagctcttcttcttctttccaaacaatgcagcctgttgctaagaacggagtcagaacacaaggaggagggtttgtgtcgagaaatggaccgccaatgaggagtttgtctagcttaaacttgcctcaagcatccccttatcgttatgcccgtcaatcaccaaagcctaaaggaaaagagcca

SEQ ID NO: 19 - protein, 1176 aa, Arabidopsis thaliana
MSITCELLNLTSKKAKKSSSSDKKWLKKPLFFLILCGSLVIVLVMFLRLGRSQKEETDSCNGEEKVLYRHQNVTRSEIHDLVSLFSDSDQVTSFECHKESSPGMWTNYGITCSLSVRSDKQETRGLPWNLGLGHSISSTSCMCGNLEPILQQPENLEEENHEEGLEQGLSSYLRNAWWCLILGVLVCHKIYVSHSKARGERKEKVHLQEALAPKKQQQRAQTSSRGAGRWRKNILLLGILGGVSFSVWWFWDTNEEIIMKRRETLANMCDERARVLQDQFNVSLNHVHALSILVSTFHHGKIPSAIDQRTFEEYTERTNFERPLTSGVAYALKVPHSEREKFEKEHGWAIKKMETEDQTVVQDCVPENFDPAPIQDEYAPVIFAQETVSHIVSVDMMSGEEDRENILRARASGKGVLTSPFKLLKSNHLGVVLTFAVYDTSLPPDATEEQRVEATIGYLGASYDMPSLVEKLLHQLASKQTIAVDVYDTTNTSGLIKMYGSEIGDISEQHISSLDFGDPSRNHEMHCRFKHKLPIPWTAITPSILVLVITFLVGYILYEAINRIATVEEDCQKMRELKARAEAADIAKSQFLATVSHEIRTPMNGVLGMLKMLMDTDLDAKQMDYAQTAHGSGKDLTSLINEVLDQAKIESGRLELENVPFDMRFILDNVSSLLSGKANEKGIELAVYVSSQVPDVVVGDPSRFRQIITNLVGNSIKFTQERGHIFISVHLADEVKEPLTIEDAVLKQRLALGCSESGETVSGFPAVNAWGSWKNFKTCYSTESQNSDQIKLLVTVEDTGVGIPVDAQGRIFTPFMQADSSTSRTYGGTGIGLSISKRLVELMQGEMGFVSEPGIGSTFSFTGVFGKAETNTSITKLERFDLAIQEFTGLRALVIDNRNIRAEVTRYELRRLGISADIVSSLRMACTCCISKLENLAMILIDKDAWNKEEFSVLDELFTRSKVTFTRVPKIFLLATSATLTERSEMKSTGLIDEVVIKPLRMSVLICCLQETLVNGKKRQPNRQRRNLGHLLREKQILVVDDNLVNRRVAEGALKKYGAIVTCVESGKAALAMLKPPHNFDACFMDLQMPEMDGFEATRRVRELEREINKKIASGEVSAEMFCKFSSWHVPILAMTADVIQATHEECMKCGMDGYVSKPFEEEVLYTAVARFFEPC

SEQ ID NO: 20 - protein, 1036 aa, Arabidopsis thaliana
MSLFHVLGFGVKIGHLFWMLCCWFVSWFVDNGIEDKSGLLVGSVGDLEKTKMTTLKKKNKMWFWNKISSSGLKIPSFSYQFLGSVKFNKAWWRKLVVVWVVFWVLVSIWTFWYFSSQAMEKRKETLASMCDERARMLQDQFNVSMNHVQAMSILISTFHHGKIPSAIDQRTFSEYTDRTSFERPLTSGVAYAMRVLHSEREEFERQQGWTIRKMYSLEQNPVHKDDYDLEALEPSPVQEEYAPVIFAQDTVSHVVSLDMLSGKEDRENVLRARSSGKGVLTAPFPLIKTNRLGVILTFAVYKRDLPSNATPKERIEATNGYLGGVFDIESLVENLLQQLASKQTILVNVYDITNHSQPISMYGTNVSADGLERVSPLIFGDPLRKHEMRCRFKQKPPWPVLSMVTSFGILVIALLVAHIIHATVSRIHKVEEDCDKMKQLKKKAEAADVAKSQFLATVSHEIRTPMNGVLGMLHMLMDTELDVTQQDYVRTAQASGKALVSLINEVLDQAKIESGKLELEEVRFDLRGILDDVLSLFSSKSQQKGVELAVYISDRVPDMLIGDPGRFRQILTNLMGNSIKFTEKGHIFVTVHLVDELFESIDGETASSPESTLSGLPVADRQRSWENFKAFSSNGHRSFEPSPPDINLIVSVEDTGVGIPVEAQSRIFTPFMQVGPSISRTHGGTGIGLSISKCLVGLMKGEIGFSSTPKVGSTFTFTAVFSNGMQPAERKNDNNQPIFSEFRGMKAVVVDHRPARAKVSWYHFQRLGIRVEVVPRVEQALHYLKIGTTTVNMILIEQEIWNREADDFIKKLQKDPLFLSPKLILLANSVESSISEALCTGIDPPIVIVKPLRASMLAATLQRGLGIGIREPPQHKGPPALILRNLLLGRKILIVDDNNVNLRVAAGALKKYGADVVCAESGIKAISLLKPPHEFDACFMDIQMPEMDGFEATRRIRDMEEEMNKRIKNGEALIVENGNKTSWHLPVLAMTADVIQATHEECLKCGMDGYVSKPFEAEQLYREVSRFFNSPSDTES

SEQ ID NO: 21 - protein, 1080 aa, Arabidopsis thaliana
MRRDFVYNNNAMFNPLTTHYSSDMNWALNNHQEEEEEPRRIEISDSESLENLKSSDFYQLGGGGALNSSEKPRKIDFWRSGLMGFAKMQQQQQLQHSVAVKMNNNNNNDLMGNKKGSTFIQEHRALLPKALILWIIIVGFISSGIYQWMDDANKIRREEVLVSMCDQRARMLQDQFSVSVNHVHALAILVSTFHYHKNPSAIDQETFAEYTARTAFERPLLSGVAYAEKVVNFEREMFERQHNWVIKTMDRGEPSPVRDEYAPVIFSQDSVSYLESLDMMSGEEDRENILRARETGKAVLTSPFRLLETHHLGVVLTFPVYKSSLPENPTVEERIAATAGYLGGAFDVESLVENLLGQLAGNQAIVVHVYDITNASDPLVMYGNQDEEADRSLSHESKLDFGDPFRKHKMICRYHQKAPIPLNVLTTVPLFFAIGFLVGYILYGAAMHIVKVEDDFHEMQELKVRAEAADVAKSQFLATVSHEIRTPMNGILGMLAMLLDTELSSTQRDYAQTAQVCGKALIALINEVLDRAKIEAGKLELESVPFDIRSILDDVLSLFSEESRNKSIELAVFVSDKVPEIVKGDSGRFRQIIINLVGNSVKFTEKGHIFVKVHLAEQSKDESEPKNALNGGVSEEMIVVSKQSSYNTLSGYEAADGRNSWDSFKHLVSEEQSLSEFDISSNVRLMVSIEDTGIGIPLVAQGRVFMPFMQADSSTSRNYGGTGIGLSISKCLVELMRGQINFISRPHIGSTFWFTAVLEKCDKCSAINHMKKPNVEHLPSTFKGMKAIVVDAKPVRAAVTRYHMKRLGINVDVVTSLKTAVVAAAAFERNGSPLPTKPQLDMILVEKDSWISTEDNDSEIRLLNSRTNGNVHHKSPKLALFATNITNSEFDRAKSAGFADTVIMKPLRASMIGACLQQVLELRKTRQQHPEGSSPATLKSLLTGKKILVVDDNIVNRRVAAGALKKFGAEVVCAESGQVALGLLQIPHTFDACFMDIQMPQMDGFEATRQIRMMEKETKEKTNLEWHLPILAMTADVIHATYEECLKSGMDGYVSKPFEEENLYKSVAKSFKPNPISPSS

SEQ ID NO: 22 - protein, 974 aa, Zea mays
MGGKYRAARTKRWWRGLAAAGWVLTAVVCSAVMHWTLRRDSMDRAEERLVSMCEERARMLQEQFGVTVNHVHAIAILISTFNFEKSPPAIDQDTFAKYTARTSFERPLLNGVAFAQRVFHHEREMFESQQGWVMNTMQREPAPPQVEYAPVIFSQDTVSYLARIDMMSGEEDRENIFRARTTGKAVLTNPFRLLGSNHLGVVLTFAVYRPDLPADASVEQRVEATIGYLGGAFDVESLVENLLSKLAGNQDIVVNVYDVTNASDAMVLYGPSSLDEQVPFLHVSMLDFGDPFRKHEMRCRYRQKLPMPWSAITNPLGTFVIWMLLGYSIAAAYSRYDKVTEDCRKMEELKTQAEAADVAKSQFLATASHEIRTPMNGVLGMLDMLLGTDLTMTQKDYAQTAQMCGRALITLINDVLDRAKIEAGKLELEAVPFDLRSLMDDVVSLFSSKSREKCIELAVFVCDNVPKVVIGDPWRFRQILTNLVGNAVKFTERGHVFVRVCLAENSNMEANQVLHGAMNGKGGRVESTANGAFNTLSGFEAADRRNSWQYFKLLLSDKESLLDDLESENSNQSDSDRVTLAISIEDTGVGIPLQAQDRVFTPFMQADSSTSRNYGGTGIGLSISKCLAELMGGQISFTSHPSVGSTFTFSATLKHSHKDISGDSSRSLTEALPTAFKGMKAILVDGRPVRSAVTRYHLKRLGILLQVVNNMNAVVKAFPGQNGAAGSREKASILFIESDFWRPETDVQLLNHLREQKNGQLSDGHKVVLLVTSEADKDKYGSIFDIVMCKPIRASTIASSIQQLLKVEIAERKDNQNRPSFLRSLLVGKNILVVDDNKVNLRVAAAALKKYGANVSCVESGKDAISLLQPPHRFDACFMDVQMPEMDGFEATGQIRQMELKANEERKNKLASIEGSTTAEYHLPVLAMTADVIQATYEECIKSGMDGYVSKPFDEEQLYQAVSRLVVGTTDSAV

SEQ ID NO: 23 - protein, 968 aa, Oryza sativa
MGDEYLAEPEDEVAISMWPENIGDKHQKQFKMEKLGKDQDALEDANFQQKPSSVDLNRLMELANSEKGVSQMQYFVKHWEYKRANTARLLKEQIGLLCQQRKEIEQRKQQILEEQQFQDESYYAVKWQVPILDEVYKDEWKRPSKKNDDLSHNQELKIDAEYDSISYWKERAMQLEKTLEASLQRERSLEEKLEENIKNLQSHTPVEEFSGMLKRADYFLHLVLQSAPIVIAHQDADLRYRFIFNHFPTLADEDVIGKTDYEILSGEGIEEMNNVKKEVMASGKATKREFVFNTPLFGAKTFVTYIEPVFSKSGETIGVNYVAMDITDQVTRREKMADIRVREAVQKAKETELSKSLHITEETMRAKQMLATMSHEIRSPLSGVLSMAEILATTKLDKEQYQLLEVMLSSGDLVLQLINDILDLSKVESGAMKLEATTFRPREVVKHVLQTAAASLKKELILEGCIGDNVPLEVTGDVLRIRQILTNLISNAVKFTHEGKVGINLHVLDKQLPGCRIEGGQLHSKAHSAPAAAAEHFSASPRKCDNDTLGCSNHEDACQTGIPSNDNFGEHHEGDEVVWLRCDVYDTGIGIPEKSLPLLFKRYMQASDDHARKYGGTGLGLAICKQLVELMGGTLTVVSKENEGSTFSFVLPCKIPVKEDHSDDPDDMPSSGGDFTTSDIEGSFIFKPQARPYLLTSGVSVMNNTKLIGGNQFYDPPNILEDRKPFSNGFVLAEDHSTNSASTAHQSNGPSVSRTNKEQHDNAMVIELNRQAERVSSSRGDTTSVSGLIHDERGPCRVHEEKSLHKKSKCSPSSNKAKILLVEDNKVNIMVAKSMLEQLGHGIDIVNNGLEAIRAIQKRQYDIILMDVHMPEMDGLQATKFIRSFENTGCWDTSVKPEHDQIIAGSDNLSDCAHMKKQGKRVPIIAMTANSFSESAEECLAAGMDSYISKPVNFQNIKECLQQYLPPQ

SEQ ID NO: 24 - protein, 1122 aa, Arabidopsis thaliana
MMVKVTKLVASRPIVVFCVLAFLVVVFECIWISNWRTTTENLVKEVASFTEDLRTSLVSEIENIGKFTYAKTNLSTIGLARVIDSYITNNDTGFTEIQTQIAPLLFVAYSTILQVSQVSYISRDGLMFSYIAESNTSVAVFANSSSNSSRGDYTWYTQTVDQLTGRLNGNSTKSQSLDVTHTDWFQAAQSNNYTTAFVGTSLGGEDNETLIQSVVSLYSKKGLVSLGFPVKTLTEVLNSLNLHGEELYMWTKDGTVLVREGSLNDSFFISNGSICFGRESNSLWSQCIPENCSSSGYEVEIKRLRYQAFCSVIEVSGVPLRYTLMFPNKGGATRIKHQAEKAKYQLIVVMIFLGFGWPVWFVWFMMQATRREMHMRATLINQMEATQQAERKSMNKSQAFANASHDIRGALAGMKGLIDICRDGVKPGSDVDTTLNQVNVCAKDLVALLNSVLDMSKIESGKMQLVEEDFNLSKLLEDVIDFYHPVAMKKGVDVVLDPHDGSVFKFSNVRGDSGRLKQILNNLVSNAVKFTVDGHIAVRAWAQRPGSNSSVVLASYPKGVSKFVKSMFCKNKEESSTYETEISNSIRNNANTMEFVFEVDDTGKGIPMEMRKSVFENYVQVRETAQGHQGTGLGLGIVQSLVRLMGGEIRITDKAMGEKGTCFQFNVLLTTLESPPVSDMKVRQEIEAGGDYVSTPNLGLTINTSLGGSMNIRNLSPRFNNCLSSSPKQEGSRVVLLLKNEERRRVTEKYIKNLGIKVTVVEKWEHLSYALERLFGFSPQSSMGRAECSLSCPSSRELPFIGMDGIDSRSQLPKRRSISFSAVVLLVIDAKTGPFFELCDIVKQFRRGLPHGISCKVVWLNESSTRVSERGDISCSRPLHGSRLMEVLKMLPEFGGTVLKEPPTELQRESLLRHSFVAERSPKHKVQEEGPSSMFNKKLGKRIMASTDSESETRVKSVRTGRKPIGNPEDEQETSKPSDDEFLRGKRVLVVDDNFISRKVATGKLKKMGVSEVEQCDSGKEALRLVTEGLTQREEQGSVDKLPFDYIFMDCQMPEMDGYEATREIRKVEKSYGVRTPIIAVSGHDPGSEEARETIQAGMDAFLDKSLNQLANVIREIESKRH

SEQ ID NO: 25 - protein, 1207 aa, Arabidopsis thaliana
MRGDSFSMSIENLPDSPMGSRKKKMQIRKVFDKMTEWVTPWRSNLESPREMMILRGDVEQDEFQYASSHCLSSYYSVFVVRLAIMVMLAILIGLLTVLTWHFTRIYTKQSLQTLAYGLRYELLQRPVLRMWSVLNTTSELTTAQVKLSEYVIKKYDKPTTQEELVEMYQAMKDVTWALFASAKALNAITINYRNGFVQAFHRDPASSSTFYIFSDLKNYSISGTGPEDVSGWNNKSIHGNMSAIWYQQQLDPVTGENLGKPLKIPPDDLINIAGISQVPDGEASWHVTVSKYMDSPLLSAALPVFDASNKSIVAVVGVTTALYSVGQLMRDLVEVHGGHIYLTSQEGYLLATSTDGPLLKNTSNGPQLMKATDSEEWVIKSGAQWLEKTYGSKRPHVVHAENVKLGDQRYYIDSFYLNLKRLPIVGVVIIPRKFIMGKVDERAFKTLIILISASVCIFFIGCVCILILTNGVSKEMKLRAELIRQLDARRRAEASSNYKSQFLANMSHELRTPMAAVIGLLDILISDDCLSNEQYATVTQIRKCSTALLRLLNNILDLSKVESGKLVLEEAEFDLGRELEGLVDMFSVQCINHNVETVLDLSDDMPALVRGDSARLVQIFANLISNSIKFTTTGHIILRGWCENINSLHDEMSVSVDRRKPWAPMKTKQVQHRNHLQKSCKNANKMVLWFEVDDTGCGIDPSKWDSVFESFEQADPSTTRTHGGTGLGLCIVRNLVNKMGGEIKVVQKNGLGTLMRLYLILSTPDTVDQNIQPDFSKYGLVVMLSMYGSTARMITSKWLRKHGIATVEASDWNELTQIIRDLLETGSRDNSFDSQHNISDPLRAELSNIVEIKNPVFVIVVDIGVLDLTTNIWKEQLNYLDRFSNKAKFAWLLKHDTSNTVKTELRRKGHVMMVNKPLYKAKMIQILEAVIKNRKRGLCNDLRNRGNGSDESHDCLEIDPTQFDTCSSDDSSETSGEKQVDKSVKPSTLHSPVLKNYLIDATTSNDDSTSASMTQKNPEEEDWKDRLYSGIALDGKNQKSLEGIRFLLAEDTPVLQRVATIMLEKMGATVTAVWDGQQAVDSLNYKSINAQAPTEEHKSFEEETANKVTTRETSLRNSSPYDLILMDCQMPKMDGYEATKAIRRAEIGTELHIPIVALTAHAMSSDEAKCLEVGMDAYLTKPIDRKLMVSTILSLTKPSAFQTSLSA

SEQ ID NO: 26 - DNA, 3240 bp, Zea mays
  CDS (1)...(3180): ZmCKI1 full-length
atgacgactgttgaaacgaatagagaagcaaaaaaatgtcttctcggtcagtacttgtgtatgtttttgcctctggcggtcgccggagtcgtcacggtaccaagtcttgtgatgcttttgctctccttgagggaaacagcgagaactaaagatgacgtcaacgtagagagcatgggtctggcgctccggcacgtcgccggcaacttgcagccgctcctcgaagccaaccgctccgccgccgccatcgccgacgccatgcggaaccccgcgagcagcaatgaatcatcaccggcttccatctctcaagtgaggcccaagatgttcatggccttcgccatgcagccgctgctggcgcaggtgtcgtacgctggcgccgacggccacgcgttcgccttctaccgcacggagcacggcgaagcgagagcgctgttcacggactcgcagaagaggtggtgcacccaggcggtggacccggcgaccggccgcttagtcgggtcggcagccgtcgccgctagcaaggacgccgccctcgtcgcgagcaagaacgcctcgcccgcgtcactccgggcagggtgggcgcgcccggacgtccggatgctcttcttctccgcgcccgtcggcgacgccgccggcgtggtctcggcagcgatcgccgtccacgatgtattcgaggccgccgccgcccgcatcggcgtccaggacggcctgaacgtgtactacgcggtcggcgacaaggtggcgtcagcggcgaccaccaactacaaacctctgtcgcttggctaccagtacggcacagccgaagacacggcggaggagaagctctcgttctctaggctgacgtgcgccgcctccgccattgacgatgccacgcccgagctcggccagctcgtcccagggggccccaagtaccggcggtacaaagccgcgtgcaccacgttcgacgtctccggagtacaagtggcactccggctggtactccgcccctcgatccagggccacatgctccaggcgttgtgcgtgtctgtggtcttcttagcgtgcgtcctggtgctggcggctgccgtgtcgtgcgccctcgcggtgcgggcgctgcggtgggcggcggcacgggagatggcgctgaacgtcgccctagcccgacagaaggaggcactgcggcaggcggagttcaagagcaatgccttcgccagtgccagccatgacatccggtccgcgctgtccgccatcgccggcctcgtggacatgtcccgtccggaggtgcagtcggtccccggcatcatggaaaaccttgatcagatgggtgtctgcaccaacaaactgttcgatattctaaactcggttctagacacaagcaaggtgggggcggggaagatgcagttagaggaagccgagttcaccatggcggacgtcctgcaggagtcggtggacatggccaacgtgacgggcgtccagcgaggggtcgaggtggtctgggacccctgcgacttctcggtcttgagatgcacagccgtcatcggagacagcaagcgcctcaagcagatcctggacaacctgcttggcaacgccctcaagttcaccgatgaaggacacgtcgtactccggtgctgggcaacccggccgatcgtcgggagcaatgtcagagctccatcaaggtttatgcacccacgacatgccagcggcggttccttcggatgcttcttcagggcacaggaggatcctagtcaccaagatcatttgcagaatgaccccaatttggttgagttttactttgaagtggttgacactggcattgggatccccaaggagaataggatgtctgtgttcgaaaactatgttcaggtgaacaatggccaaggaggcacaggcttggggctcggaattgtgcaatcctttgtacgtctgatgggaggagagattagcatcaaggacaaagagcctggggaaaggggaacatgcttcgcattcaacgtgctcctaaagataagcgagatgcaacaacctcaagacatagaagaaggaccatcggttccatcaggtacactgaacctagcctcagcgtttcaagaagccaacaacttcaagggtgtgcattgcattctgtacgttcgtgggtatgaaaccaggaggatcatgcagacatggatggagagcattggtgtcaaggtctggctggttccgcaggctaagttcatcggctccaccttggagaaggtgcggagcaatagcacaaccatcattgccatggactacaacaacgccgaccgatgcttcagcttcaaggagaaggtgaaccagctgaggaacaacactggtatcaggagagtcagccttggggggattccttccggtatccttattgtcatcgatgtgtccggtggtgaaacggaagaaattttccaggagatggccaaacttgccagaatcaagcatcaagccccatgcaaagttgttttgctcaaggatatcaagaccccttccaatgatctgaggaagctcaaggacctaggttgcgatctcgttttgcgaaagccagtgcacgggtctcgccttttcactctccttatgaccttgagagacctccaggtttcaaatgcacaagcacagtcttctcatgttgttggccctgggattgctgggaacagccagcagcacggtttgccagatgttgttgttccttgcgtgcaagaaactaatgcatcaactgaattagcttcatgcttggctcaggagcagaagcctgaagatgataagccgctaagcgggatgcaaatcttgctggctgaagacactctagtgctgcaaacaatccaaaagaaagtcctaaaccagcttggagcacttgtcacagtagctcaaaatggagctgtggctgtgaacttgctcagagaagctcttgagcaagccaatgcttctaaagaggatatagtgtccttgccctaccatgtcatcttaatggattgccagatgcccgaaatggatggctatgaagcaacgaaactcatccgtgaggaagaacaacgctacggaattcacactccgattatcgcattaaccgcgcatgacatggaggaggatctgcagaaggcttttgatgcagggatggatcttcacctgacaaagccaatattgcagaagaagattgtggaagctatttgcagtgtttataaaggtcagaattgaatgacgactgttgaaacgaatagagaagcaaaaaaatgtcttctcggtcagtacttgtgt

SEQ ID NO: 27 - protein, 1059 aa, Zea mays
MTTVETNREAKKCLLGQYLCMFLPLAVAGVVTVPSLVMLLLSLRETARTKDDVNVESMGLALRHVAGNLQPLLEANRSAAAIADAMRNPASSNESSPASISQVRPKMFMAFAMQPLLAQVSYAGADGHAFAFYRTEHGEARALFTDSQKRWCTQAVDPATGRLVGSAAVAASKDAALVASKNASPASLRAGWARPDVRMLFFSAPVGDAAGVVSAAIAVHDVFEAAAARIGVQDGLNVYYAVGDKVASAATTNYKPLSLGYQYGTAEDTAEEKLSFSRLTCAASAIDDATPELGQLVPGGPKYRRYKAACTTFDVSGVQVALRLVLRPSIQGHMLQALCVSVVFLACVLVLAAAVSCALAVRALRWAAAREMALNVALARQKEALRQAEFKSNAFASASHDIRSALSAIAGLVDMSRPEVQSVPGIMENLDQMGVCTNKLFDILNSVLDTSKVGAGKMQLEEAEFTMADVLQESVDMANVTGVQRGVEVVWDPCDFSVLRCTAVIGDSKRLKQILDNLLGNALKFTDEGHVVLRCWATRPIVGSNVRAPSRFMHPRHASGGSFGCFFRAQEDPSHQDHLQNDPNLVEFYFEVVDTGIGIPKENRMSVFENYVQVNNGQGGTGLGLGIVQSFVRLMGGEISIKDKEPGERGTCFAFNVLLKISEMQQPQDIEEGPSVPSGTLNLASAFQEANNFKGVHCILYVRGYETRRIMQTWMESIGVKVWLVPQAKFIGSTLEKVRSNSTTIIAMDYNNADRCFSFKEKVNQLRNNTGIRRVSLGGIPSGILIVIDVSGGETEEIFQEMAKLARIKHQAPCKVVLLKDIKTPSNDLRKLKDLGCDLVLRKPVHGSRLFTLLMTLRDLQVSNAQAQSSHVVGPGIAGNSQQHGLPDVVVPCVQETNASTELASCLAQEQKPEDDKPLSGMQILLAEDTLVLQTIQKKVLNQLGALVTVAQNGAVAVNLLREALEQANASKEDIVSLPYHVILMDCQMPEMDGYEATKLIREEEQRYGIHTPIIALTAHDMEEDLQKAFDAGMDLHLTKPILQKKIVEAICSVYKGQN

SEQ ID NO: 28 - DNA, 3180 bp, Zea mays
  CDS (1)...(3180): ZmCKI1
atgacgactgttgaaacgaatagagaagcaaaaaaatgtcttctcggtcagtacttgtgtatgtttttgcctctggcggtcgccggagtcgtcacggtaccaagtcttgtgatgcttttgctctccttgagggaaacagcgagaactaaagatgacgtcaacgtagagagcatgggtctggcgctccggcacgtcgccggcaacttgcagccgctcctcgaagccaaccgctccgccgccgccatcgccgacgccatgcggaaccccgcgagcagcaatgaatcatcaccggcttccatctctcaagtgaggcccaagatgttcatggccttcgccatgcagccgctgctggcgcaggtgtcgtacgctggcgccgacggccacgcgttcgccttctaccgcacggagcacggcgaagcgagagcgctgttcacggactcgcagaagaggtggtgcacccaggcggtggacccggcgaccggccgcttagtcgggtcggcagccgtcgccgctagcaaggacgccgccctcgtcgcgagcaagaacgcctcgcccgcgtcactccgggcagggtgggcgcgcccggacgtccggatgctcttcttctccgcgcccgtcggcgacgccgccggcgtggtctcggcagcgatcgccgtccacgatgtattcgaggccgccgccgcccgcatcggcgtccaggacggcctgaacgtgtactacgcggtcggcgacaaggtggcgtcagcggcgaccaccaactacaaacctctgtcgcttggctaccagtacggcacagccgaagacacggcggaggagaagctctcgttctctaggctgacgtgcgccgcctccgccattgacgatgccacgcccgagctcggccagctcgtcccagggggccccaagtaccggcggtacaaagccgcgtgcaccacgttcgacgtctccggagtacaagtggcactccggctggtactccgcccctcgatccagggccacatgctccaggcgttgtgcgtgtctgtggtcttcttagcgtgcgtcctggtgctggcggctgccgtgtcgtgcgccctcgcggtgcgggcgctgcggtgggcggcggcacgggagatggcgctgaacgtcgccctagcccgacagaaggaggcactgcggcaggcggagttcaagagcaatgccttcgccagtgccagccatgacatccggtccgcgctgtccgccatcgccggcctcgtggacatgtcccgtccggaggtgcagtcggtccccggcatcatggaaaaccttgatcagatgggtgtctgcaccaacaaactgttcgatattctaaactcggttctagacacaagcaaggtgggggcggggaagatgcagttagaggaagccgagttcaccatggcggacgtcctgcaggagtcggtggacatggccaacgtgacgggcgtccagcgaggggtcgaggtggtctgggacccctgcgacttctcggtcttgagatgcacagccgtcatcggagacagcaagcgcctcaagcagatcctggacaacctgcttggcaacgccctcaagttcaccgatgaaggacacgtcgtactccggtgctgggcaacccggccgatcgtcgggagcaatgtcagagctccatcaaggtttatgcacccacgacatgccagcggcggttccttcggatgcttcttcagggcacaggaggatcctagtcaccaagatcatttgcagaatgaccccaatttggttgagttttactttgaagtggttgacactggcattgggatccccaaggagaataggatgtctgtgttcgaaaactatgttcaggtgaacaatggccaaggaggcacaggcttggggctcggaattgtgcaatcctttgtacgtctgatgggaggagagattagcatcaaggacaaagagcctggggaaaggggaacatgcttcgcattcaacgtgctcctaaagataagcgagatgcaacaacctcaagacatagaagaaggaccatcggttccatcaggtacactgaacctagcctcagcgtttcaagaagccaacaacttcaagggtgtgcattgcattctgtacgttcgtgggtatgaaaccaggaggatcatgcagacatggatggagagcattggtgtcaaggtctggctggttccgcaggctaagttcatcggctccaccttggagaaggtgcggagcaatagcacaaccatcattgccatggactacaacaacgccgaccgatgcttcagcttcaaggagaaggtgaaccagctgaggaacaacactggtatcaggagagtcagccttggggggattccttccggtatccttattgtcatcgatgtgtccggtggtgaaacggaagaaattttccaggagatggccaaacttgccagaatcaagcatcaagccccatgcaaagttgttttgctcaaggatatcaagaccccttccaatgatctgaggaagctcaaggacctaggttgcgatctcgttttgcgaaagccagtgcacgggtctcgccttttcactctccttatgaccttgagagacctccaggtttcaaatgcacaagcacagtcttctcatgttgttggccctgggattgctgggaacagccagcagcacggtttgccagatgttgttgttccttgcgtgcaagaaactaatgcatcaactgaattagcttcatgcttggctcaggagcagaagcctgaagatgataagccgctaagcgggatgcaaatcttgctggctgaagacactctagtgctgcaaacaatccaaaagaaagtcctaaaccagcttggagcacttgtcacagtagctcaaaatggagctgtggctgtgaacttgctcagagaagctcttgagcaagccaatgcttctaaagaggatatagtgtccttgccctaccatgtcatcttaatggattgccagatgcccgaaatggatggctatgaagcaacgaaactcatccgtgaggaagaacaacgctacggaattcacactccgattatcgcattaaccgcgcatgacatggaggaggatctgcagaaggcttttgatgcagggatggatcttcacctgacaaagccaatattgcagaagaagattgtggaagctatttgcagtgtttataaaggtcagaattga

SEQ ID NO: 29 - DNA, 4047 bp, Arabidopsis thaliana
  promoter (1)...(4047): AtCKI2 promoter
tgctgcttcatcgattagcccattactaactgctcgaaacagaaacagatagatgtatgaagttttgttctctatggcatactgtggttcgatcatatccttcatgaatttacgcacttgaaagtcaaaagtggctgtaaatcaactaaacttatctacaatgagaagagatatacatttgttttcttggtccaccatttagcaatttttcatattaaagatcctagttgaagtaattaaaaacgaaaagtatgttactaaatatgaaattacggtgatggtaaggaatcccggagttgtgttacccactttgaattatcttttcacaaatccatccatgcaaaaactttaagagtcactacttatttttgttgctgttccttcccccatcacttgtcatatcacaatttaagagattatgctccttaaacatatcggaacttaacatagaaagttcttgataaatgaaaaatttgttcattgtttcaattatttttactgtatgaaagaattcaaatgggtttgtacttggcaaattactttttgtatttttcaatgagttttttctcaaaaaaaattagtagtaatactcaatatatctacatcgaataagtgacacaaacaaaaatcaactcaattcttctctagattatggccggactatcagacttaggatcataataagagaatgtacactttttccttttttataaaaccagaaaacagtacattggagatttactttagatctatcttcctgagtgatttattattggaacatatgattaagacactgaaaagttccttgtcagaaatataaatgtaaagaactctgaccagggccctacacgggatagattatcgaattttattttaaccctttttaataatggaacccaaaaaagtagtatcaaataatactaaaatcgactttaaagtaatactaatccaagtgttattatgaaactactgaaataccgaaaattatggtatagccacacactaatagaacctcttaaacatgtcatcttatcatccatctaaccatcttaataagttatttattctgttggaaaactaaaatatagtttgtatgttactcttaagagattcacggatgacatttccactcggaaattattataaattctaacaatataatattattgaattaacaaaatgaatctatatggcaccgagtcattcataacaacaatatatatgcccccaactattgactacaataaatcgacaaccaatttgcaagttttttttttacttaatcatatgccaccacgacgtaatccatgatgggttagtccaaggaggtaaaactttcgtgagatggtaaatttctctaataatcttaaagtagtcataatgtcaaacccaactttgcacacacatgtattatgtatatgaatataatataataatgtactaagtaaataattaggaagaaagctgaaaagtatggtcctgctttaatcgagaattaaacaaatccggtatttaaggattggtgtccccatccaattaatatctccacattctcgtcttttcgtccaaaactaaaaaaaaataacaatatccttaatctccttcaccaccgtccataatttttggcgcgagagcaacaaaaaaaaaaaaacacgacaaacagaagaaacttgtttgaaaatgattagaccacgtgttagtcaacggtaattccacgtgtaacattgaggctgacgtggaacatgtgactaaccacatcacatgtttcctattagccacttacggacgggacaaaagcctccgagcgccgttcagatcgggacttaagacaccaaaaatggcttagctgttttcttaaattattttaatttaaagaccctttaatttattattattagttttgattattgtgtgtactattaccactagttgctaattttttagtatttaatttattgatatagaaatgggattgatttcagacaactaattttgtgccaatacagttgttgtccgctccattgaattgtgaggattgggtcttattcattttttctattagcttttctcgtgtttttttatgcgcaaggtgttcaaattattgatcatactagtttgcaactgaaattgaagcgtagaatatagtttaagatttgggtgattaatacaaatattatactactagttacgatacatttatttgtggtactacatgtgctttctgctattcatgaaacaatgaatgtcccgacccaattcgaatatttgtgtactttcgtcaacattgacattctatagactatatattagaaccacatgattatacattttaaaaaaaaatcttcttttttcgtaatgagtagtagtacaaaaggcaaaagaaataaatcatgcattcgttacttattgacaactagaaataactataattatgtttagttattttcattatcggatcatcaaatcatctaatatatttttccaaaagcttaacattctacaaaactttacaacacaaaaatttatatatatgattcaacttaaaaattcatgagctaaacagagttgctctgtttcggaaaatcattctaattacgtagaataaaactcacttatataaatctactaatcaaagagttgtccagtttactgattaagtagccaaatcatgaattaatagtgatcgtcgtcgagcaaattaaaatccaaatctataacaattattgacgccacttctagttactcctcaataaaggaagttgctaatacttaattaaaaaatagtgtgactatcctcaatatactcggagcgtgacgtaaacgcgccgccttatagcttccgtcgtcaactctgtgaggtataaaagacgtaaggaaaagcagagaaactggaggaaaaaaaagaacagagagaaagagagaagagtgaattgtgagacagatcgaaacgtgtcaatctgtgtgtcattcgatagtaactcggattcggtctcagaaagaaaacaaacgtgcacacacagagcagagagacagagtcgccattaaaatatgtccaacttaaaaatgtgagaaagagagagaagagaagagaagagaagagatgaatataatatacattgacctctctgtctcatgagcaagcacatgtcctctctctctctctctttctcttctccgtaaaaaaaattaccaatctttcacaattcattcacatgctcctctctctttcttcattggctattctcttcccaatgctcatttgaagtttatttactctactcccgacgaccacgactcttcttcagatctactcactccttctttctctccttcttcttcttcatttttccggtgaccggagtcggagaaggtaaagcttctgatcccttcccctttcctccggcatactcgttcctctgctttgtctttcgtttttttcatttctttctgtttcagccttttgcatctcgagacttcatgattacaatttctttatgtttctcaatgcaagattttcgtttataatatatgatcactgatgtcaagaataaatcaccaaagatttttttctttccatatattttttccgatcatcgctcaaaaaaaagtttcatcgtttaaatttattttcagattttatcgtttattggtgaattttatgatcctagttgataattcaatctgaaaaaagaaactgatacagttttctttttgaagctctgcaaatttctgatttttaagctcgaatcatataatttgaagtttcccgctaatgttcaatcagaattggtccacacacttagttgttgtggaccctttgcaaaattctattttagttcattatttttcacattttacagatcaagattctctgatggagatggtttgtcctgactctaacctctgtgtttgaaaatatctatacaggttctttattcagatcaaggttctggcttaaagaaaaaagttgtttgaattttgagatttgtctggtccattgtgttgctgttgttgtatgaagagaaaccttgatcaatggtctgtgaa

SEQ ID NO: 30 - DNA, 3606 bp, Zea mays
  CDS (1)...(3606): ZmHK3
atggttccgaacggagcaggggaatgcggcggcggctgccggcgagggaaagcagcgcacgccggtgtcggcgctgtggctggtttcttcttatgtctcttgatggtctgggcagttggtgggtctgaaagatcaggttgtggccgggtggctgagcaccacggggtggtacgggtggggcagttcaacctcagcaggagccagctgcaggcgttggtgttcctcctctcatcgacacataaagaatgtatggtaaattctggaatggtccatgatgggaatcaagtcaatggtgcgtcttttatcccagatattcgttgcattcgagataggatatatgatggaaaacagaattggttgaaggatgcaattcttcaagaatcttgcacagcacaagataaatatggcctaaatagtcatgcacctgcagtattggaagataagttattgcaaaatgttatacaagaagacattaattctaccacgcagtgcaatccggacgaatgtgacttatgtgctggaatcagtggcgtgaatgtggtccagaatattgggtcaagttctaatcgcacaataatattatgtctatctgcttttttggggagcattgtgatatcaatagttaaaactacgcacaaaagagggattcaaacccacaagctttatgcagatgagaaagttctccaggcaccaagtactaaaataagtcgaaagtggtcaaagagagccctgcttattggtgtcttatttgggctctgtagctctgtatgcatattttcatgcatgtatgccgatgttgttgctcggagaattgagaatcttgctaatatgtgtgatgagagagcaaggatgttacaggatcagtttaatgtaagcatgaaccatgtgcatgcgttggctatactggtctcatcatttcaccatgggaaggatccttctgctattgatcagaaaacatttgaggacttcacagcaaggacaacatttgaaaggccattgatgagtggtgtcgcctatgcattgaaggtattacacaatgaaagacaacagtttgaggaacaacatggatggaagattaagaagatggaagatggagatcagtcccttgttcatgactacaaccttgaaaagcttgaaccatcaccagttcaagatgagtatgcgcctgttattttctctcaggaaactgtgaagcacattatatctgtagatatgatgtctgggaaggaagatcatgacaatatattacgttcccggtcaacaggtaaaggggccctaacatccccttttaagcttctgaagtccaaccatctcggtgtggtgcttacattcactgtatataaatatgatctgcctccaaatgcaacaccacaggagcgtattcatgctacattgggctatcttggtgcctcatttgatgtcccttctctggttgataaactcctagaacaactcgctagcaaacgaaagttagtagttaatttatatgacaccacaaatcatacctctcctattgaaatgtatggttctgattttactccatctggtgatctgcatattagtagcattgattttggagacccaacacgcaaacatgagatgcattgcagatttaaacatgaaccttcactgccatggtctgcaataataatatcaacagctgtagctataattgttttgcttgttggacatattatttatgcaactctgaactcccttgaaaaggctgaacatgattatagagtaatgagagaacttaaaggacaagctgaagctgcagatatagcaaaatctcagtttctagcaactgtttcccatgagattcggactccaatgaatggtgttttaggtatgctgcagatgctaatggacactgaacttgatacaactcagcaggattttgtagtcaccgctcaagaaagtggaaaggtattaataaacttaataaatgaagtccttgatctggctaagatagagtcaggaaggattgagctggaagcagtgccttttgatgtccgtgatattcttgacaatgtgatatctcttttctacgataaatcacaagctaaaggcatagagttggccgtgcttgtatctgaccaagttcctgatgtcctaattggggatccatggcggttccggcagatcattacgaaccttgtggggaattctatgaagtttactgaacagggccacatcttcgttcaagtccatttggtcaaggaactaaacaggaaaggaaacaatttttgtgatgtttctgctcataacagagaaattctgtatgattctgacaactcgatgctatggaacacactaagcggacttgaagtagcagatagctggaggtccttggagaattttacgatgttcaagaattcaaatggtgaaacagatacaattaggttagctgtcagagttgaagacaccggcataggaataacaaaagatgctcaaatgcgtatttttacacctttcatgcaagctgacagttccacctcacgaacatatggtggaactggaattggcttgagtattacaaaacgtttagttgaattgatgggtggagaaatagggtttactagtaaatcaggagttggtagtacattttcttttactgccattttcaaggagaacagaggatgtcctggagatatgaaaagatactattctgaacctactccgttggattttcaaggaatgagggctcttattattgatggaaggagtgcacgtgcagaggttactatgtaccatcttcgtagacttggaatacagtgcagtcttgttgccacatctgaatctgaatctgcatcttcagctttgatgaaagcctgcacctcaagcaaaaacaatccggacatggtccttgttgatacagaagcttggggtaaaggttcaggtttggcattctatcatcgttttgtagattgtcaactaaagggcgcacacaagttttctcagcccatgccaaagactattcttttgggaagctcaataagccctgttgaatctgattatttaagattgacaggatttggtgattgcataaggaaacccctccgtcttagtactattgttgcctgtcttcgcaaaaccttaggggttggggttacaaggcagcagagtagagaccaatctttggttcttcaaagtgtactaactggaaaacagattctggttgtggatgataatgcagtcaaccgtaaagttgcagctggttctttgaagaaatatggatctaaagttacatgtgttgatagtgggagtgatgcaattgatatgctcaagccacctcacacatttgatgcttgcttcatggatgttcagatgcccgagatggacgggtttgaagctacaagattaatccggtccatagagaaaaaaataaacgatatgatcatgatgggtgaaatatccgctaataattatggaaataaggcccattggcatgtaccaatattagctatgacggcagatgtaatccaagcgacttttgaaaaatgtatgcagtgtggaatggatggatatgtgtcaaagccatttgaagaacagcagctctactttgctgttgctcatttcttagagactgatgaaatagaataa

SEQ ID NO: 31 - protein, 1201 aa, Zea mays
MVPNGAGECGGGCRRGKAAHAGVGAVAGFFLCLLMVWAVGGSERSGCGRVAEHHGVVRVGQFNLSRSQLQALVFLLSSTHKECMVNSGMVHDGNQVNGASFIPDIRCIRDRIYDGKQNWLKDAILQESCTAQDKYGLNSHAPAVLEDKLLQNVIQEDINSTTQCNPDECDLCAGISGVNVVQNIGSSSNRTIILCLSAFLGSIVISIVKTTHKRGIQTHKLYADEKVLQAPSTKISRKWSKRALLIGVLFGLCSSVCIFSCMYADVVARRIENLANMCDERARMLQDQFNVSMNHVHALAILVSSFHHGKDPSAIDQKTFEDFTARTTFERPLMSGVAYALKVLHNERQQFEEQHGWKIKKMEDGDQSLVHDYNLEKLEPSPVQDEYAPVIFSQETVKHIISVDMMSGKEDHDNILRSRSTGKGALTSPFKLLKSNHLGVVLTFTVYKYDLPPNATPQERIHATLGYLGASFDVPSLVDKLLEQLASKRKLVVNLYDTTNHTSPIEMYGSDFTPSGDLHISSIDFGDPTRKHEMHCRFKHEPSLPWSAIIISTAVAIIVLLVGHIIYATLNSLEKAEHDYRVMRELKGQAEAADIAKSQFLATVSHEIRTPMNGVLGMLQMLMDTELDTTQQDFVVTAQESGKVLINLINEVLDLAKIESGRIELEAVPFDVRDILDNVISLFYDKSQAKGIELAVLVSDQVPDVLIGDPWRFRQIITNLVGNSMKFTEQGHIFVQVHLVKELNRKGNNFCDVSAHNREILYDSDNSMLWNTLSGLEVADSWRSLENFTMFKNSNGETDTIRLAVRVEDTGIGITKDAQMRIFTPFMQADSSTSRTYGGTGIGLSITKRLVELMGGEIGFTSKSGVGSTFSFTAIFKENRGCPGDMKRYYSEPTPLDFQGMRALIIDGRSARAEVTMYHLRRLGIQCSLVATSESESASSALMKACTSSKNNPDMVLVDTEAWGKGSGLAFYHRFVDCQLKGAHKFSQPMPKTILLGSSISPVESDYLRLTGFGDCIRKPLRLSTIVACLRKTLGVGVTRQQSRDQSLVLQSVLTGKQILVVDDNAVNRKVAAGSLKKYGSKVTCVDSGSDAIDMLKPPHTFDACFMDVQMPEMDGFEATRLIRSIEKKINDMIMMGEISANNYGNKAHWHVPILAMTADVIQATFEKCMQCGMDGYVSKPFEEQQLYFAVAHFLETDEIE

SEQ ID NO: 32 - DNA, 3606 bp, Zea mays; identical to SEQ ID NO: 30

SEQ ID NO: 33 - protein, 738 aa, Arabidopsis thaliana
  PEPTIDE (1)...(738): AtETR1 - AAA70047
MEVCNCIEPQWPADELLMKYQYISDFFIAIAYFSIPLELIYFVKKSAVFPYRWVLVQFGAFIVLCGATHLINLWTFTTHSRTVALVMTTAKVLTAVVSCATALMLVHIIPDLLSVKTRELFLKNKAAELDREMGLIRTQEETGRHVRMLTHEIRSTLDRHTILKTTLVELGRTLALEECALWMPTRTGLELQLSYTLRHQHPVEYTVPIQLPVINQVFGTSRAVKISPNSPVARLRPVSGKYMLGEVVAVRVPLLHLSNFQINDWPELSTKRYALMVLMLPSDSARQWHVHELELVEVVADQVAVALSHAAILEESMRARDLLMEQNVALDLARREAETAIRARNDFLAVMNHEMRTPMHAIIALSSLLQETELTPEQRLMVETILKSSNLLATLMNDVLDLSRLEDGSLQLELGTFNLHTLFREVLNLIKPIAVVKKLPITLNLAPDLPEFVVGDEKRLMQIILNIVGNAVKFSKQGSISVTALVTKSDTRAADFFVVPTGSHFYLRVKVKDSGAGINPQDIPKIFTKFAQTQSLATRSSGGSGLGLAISKRFVNLMEGNIWIESDGLGKGCTAIFDVKLGISERSNESKQSGIPKVPAIPRHSNFTGLKVLVMDENGVSRMVTKGLLVHLGCEVTTVSSNEECLRVVSHEHKVVFMDVCMPGVENYQIALRIHEKFTKQRHQRPLLVALSGNTDKSTKEKCMSFGLDGVLLKPVSLDNIRDVLSDLLEPRVLYEGM

SEQ ID NO: 34 - protein, 1220 aa, Saccharomyces cerevisiae
  PEPTIDE (1)...(1220): SLN1 - CAA86131
MRFGLPSKLELTPPFRIGIRTQLTALVSIVALGSLIILAVTTGVYFTSNYKNLRSDRLYIAAQLKSSQIDQTLNYLYYQAYYLASRDALQSSLTSYVAGNKSADNWVDSLSVIQKFLSSSNLFYVAKVYDSSFNAVLNATNNGTGDLIPEDVLDSLFPLSTDTPLPSSLETIGILTDPVLNSTDYLMSMSLPIFANPSIILTDSRVYGYITIIMSAEGLKSVFNDTTALEHSTIAIISAVYNSQGKASGYHFVFPPYGSRSDLPQKVFSIKNDTFISSAFRNGKGGSLKQTNILSTRNTALGYSPCSFNLVNWVAIVSQPESVFLSPATKLAKIITGTVIAIGVFVILLTLPLAHWAVQPIVRLQKATELITEGRGLRPSTPRTISRASSFKRGFSSGFAVPSSLLQFNTAEAGSTTSVSGHGGSGHGSGAAFSANSSMKSAINLGNEKMSPPEEENKIPNNHTDAKISMDGSLNHDLLGPHSLRHNDTDRSSNRSHILTTSANLTEARLPDYRRLFSDELSDLTETFNTMTDALDQHYALLEERVRARTKQLEAAKIEAEAANEAKTVFIANISHELRTPLNGILGMTAISMEETDVNKIRNSLKLIFRSGELLLHILTELLTFSKNVLQRTKLEKRDFCITDVALQIKSIFGKVAKDQRVRLSISLFPNLIRTMVLWGDSNRIIQIVMNLVSNALKFTPVDGTVDVRMKLLGEYDKELSEKKQYKEVYIKKGTEVTENLETTDKYDLPTLSNHRKSVDLESSATSLGSNRDTSTIQEEITKRNTVANESIYKKVNDREKASNDDVSSIVSTTTSSYDNAIFNSQFNKAPGSDDEEGGNLGRPIENPKTWVISIEVEDTGPGIDPSLQESVFHPFVQGDQTLSRQYGGTGLGLSICRQLANMMHGTMKLESKVGVGSKFTFTLPLNQTKEISFADMEFPFEDEFNPESRKNRRVKFSVAKSIKSRQSTSSVATPATNRSSLTNDVLPEVRSKGKHETKDVGNPNMGREEKNDNGGLEQLQEKNIKPSICLTGAEVNEQNSLSSKHRSRHEGLGSVNLDRPFLQSTGTATSSRNIPTVKDDDKNETSVKILVVEDNHVNQEVIKRMLNLEGIENIELACDGQEAFDKVKELTSKGENYNMIFMDVQMPKVDGLLSTKMIRRDLGYTSPIVALTAFADDSNIKECLESGMNGFLSKPIKRPKLKTILTEFCAAYQGKKNNK

SEQ ID NO: 35 - protein, 44 aa, Arabidopsis thaliana
  VARIANT (1)...(44): cki2-2 insertional mutant
GVDISTSENEQHMIYCGVNKLTLRQLNNTLRTFLMYWGGFSFHQ

SEQ ID NO: 36 - protein, 44 aa, Arabidopsis thaliana
INLIVSVEDTGVGIPVEAQSRIFTPFMQVGPSISRTHGGTGIGV

SEQ ID NO: 37 - protein, 35 aa, Arabidopsis thaliana
LSTPDTVDQNIQPDFSKYGLVVMLSMYGSTARMIT